SEQUENCE LISTING (301 sequences)

SEQ ID NO: 1 - protein, 9 aa, Artificial Sequence
  Synthetic Polypeptide
PFPQPELPY

SEQ ID NO: 2 - protein, 9 aa, Artificial Sequence
  Synthetic Polypeptide
PQPELPYPQ

SEQ ID NO: 3 - protein, 9 aa, Artificial Sequence
  Synthetic Polypeptide
PFPQPEQPF

SEQ ID NO: 4 - protein, 9 aa, Artificial Sequence
  Synthetic Polypeptide
PQPEQPFPW

SEQ ID NO: 5 - protein, 9 aa, Artificial Sequence
  Synthetic Polypeptide
EQPIPEQPQ

SEQ ID NO: 6 - protein, 9 aa, Artificial Sequence
  Synthetic Polypeptide
PIPEQPQPY

SEQ ID NO: 7 - protein, 9 aa, Artificial Sequence
  Synthetic Polypeptide
PFPQPEQPI

SEQ ID NO: 8 - protein, 9 aa, Artificial Sequence
  Synthetic Polypeptide
PQPEQPIPV

SEQ ID NO: 9 - protein, 9 aa, Artificial Sequence
  Synthetic Polypeptide
EQPIPVQPE

SEQ ID NO: 10 - protein, 9 aa, Artificial Sequence
  Synthetic Polypeptide
PFPQPEQPT

SEQ ID NO: 11 - protein, 9 aa, Artificial Sequence
  Synthetic Polypeptide
PQPEQPTPI

SEQ ID NO: 12 - protein, 9 aa, Artificial Sequence
  Synthetic Polypeptide
EQPTPIQPE

SEQ ID NO: 13 - protein, 9 aa, Artificial Sequence
  Synthetic Polypeptide
PQPEQPFPL

SEQ ID NO: 14 - protein, 9 aa, Artificial Sequence
  Synthetic Polypeptide
EQPFPLQPE

SEQ ID NO: 15 - protein, 9 aa, Artificial Sequence
  Synthetic Polypeptide
EGSFQPSQE

SEQ ID NO: 16 - protein, 9 aa, Artificial Sequence
  Synthetic Polypeptide
QGYYPTSPQ

SEQ ID NO: 17 - protein, 9 aa, Artificial Sequence
  Synthetic Polypeptide
EQPEQPFPE

SEQ ID NO: 18 - protein, 9 aa, Artificial Sequence
  Synthetic Polypeptide
PFSEQEQPV

SEQ ID NO: 19 - protein, 9 aa, Artificial Sequence
  Synthetic Polypeptide
PYPQPELPY

SEQ ID NO: 20 - protein, 9 aa, Artificial Sequence
  Synthetic Polypeptide
EQPFPEQPI

SEQ ID NO: 21 - protein, 9 aa, Artificial Sequence
  Synthetic Polypeptide
PFPEQPIPE

SEQ ID NO: 22 - protein, 9 aa, Artificial Sequence
  Synthetic Polypeptide
PYPEQPQPF

SEQ ID NO: 23 - protein, 9 aa, Artificial Sequence
  Synthetic Polypeptide
PQPYPEQPQ

SEQ ID NO: 24 - protein, 12 aa, Artificial Sequence
  Synthetic Polypeptide
PFPQPELPYPQP

SEQ ID NO: 25 - protein, 12 aa, Artificial Sequence
  Synthetic Polypeptide
PFPQPEQPFPWQ

SEQ ID NO: 26 - protein, 12 aa, Artificial Sequence
  Synthetic Polypeptide
EQPIPEQPQPYP

SEQ ID NO: 27 - protein, 12 aa, Artificial Sequence
  Synthetic Polypeptide
PFPQPEQPIPVQ

SEQ ID NO: 28 - protein, 12 aa, Artificial Sequence
  Synthetic Polypeptide
PEQPIPVQPEQS

SEQ ID NO: 29 - protein, 12 aa, Artificial Sequence
  Synthetic Polypeptide
PFPQPEQPTPIQ

SEQ ID NO: 30 - protein, 12 aa, Artificial Sequence
  Synthetic Polypeptide
PEQPTPIQPEQP

SEQ ID NO: 31 - protein, 12 aa, Artificial Sequence
  Synthetic Polypeptide
PFPQPEQPFPLQ

SEQ ID NO: 32 - protein, 12 aa, Artificial Sequence
  Synthetic Polypeptide
PEQPFPLQPEQP

SEQ ID NO: 33 - protein, 12 aa, Artificial Sequence
  Synthetic Polypeptide
GEGSFQPSQENP

SEQ ID NO: 34 - protein, 12 aa, Artificial Sequence
  Synthetic Polypeptide
QQGYYPTSPQQS

SEQ ID NO: 35 - protein, 12 aa, Artificial Sequence
  Synthetic Polypeptide
PEQPEQPFPEQP

SEQ ID NO: 36 - protein, 12 aa, Artificial Sequence
  Synthetic Polypeptide
PPFSEQEQPVLP

SEQ ID NO: 37 - protein, 12 aa, Artificial Sequence
  Synthetic Polypeptide
PYPQPELPYPQP

SEQ ID NO: 38 - protein, 12 aa, Artificial Sequence
  Synthetic Polypeptide
EQPFPEQPIPEQ

SEQ ID NO: 39 - protein, 12 aa, Artificial Sequence
  Synthetic Polypeptide
PQPYPEQPQPFP

SEQ ID NO: 40 - protein, 9 aa, Artificial Sequence
  Synthetic Polypeptide
PQPEQPFSQ

SEQ ID NO: 41 - protein, 9 aa, Artificial Sequence
  Synthetic Polypeptide
EQPFPEQPQ

SEQ ID NO: 42 - protein, 9 aa, Artificial Sequence
  Synthetic Polypeptide
PFPEQPEQI

SEQ ID NO: 43 - protein, 9 aa, Artificial Sequence
  Synthetic Polypeptide
PQPELPYPY

SEQ ID NO: 44 - protein, 18 aa, Artificial Sequence
  Synthetic Polypeptide
PEQPFPEQPIPEQPQPYP

SEQ ID NO: 45 - protein, 15 aa, Artificial Sequence
  Synthetic Polypeptide
LQPFPQPELPYPQPQ

SEQ ID NO: 46 - protein, 14 aa, Artificial Sequence
  Synthetic Polypeptide
QPFPQPEQPFPWQP

SEQ ID NO: 47 - protein, 15 aa, Artificial Sequence
  Synthetic Polypeptide
PEQPIPEQPQPYPQQ

SEQ ID NO: 48 - protein, 17 aa, Artificial Sequence
  Synthetic Polypeptide
QPFPQPEQPIPVQPEQS

SEQ ID NO: 49 - protein, 17 aa, Artificial Sequence
  Synthetic Polypeptide
QPFPQPEQPTPIQPEQP

SEQ ID NO: 50 - protein, 17 aa, Artificial Sequence
  Synthetic Polypeptide
QPFPQPEQPFPLQPEQP

SEQ ID NO: 51 - protein, 13 aa, Artificial Sequence
  Synthetic Polypeptide
QPFPQPEQPFSQQ

SEQ ID NO: 52 - protein, 14 aa, Artificial Sequence
  Synthetic Polypeptide
PQPYPEQPQPFPQQ

SEQ ID NO: 53 - protein, 15 aa, Artificial Sequence
  Synthetic Polypeptide
QPFPEQPEQIIPQQP

SEQ ID NO: 54 - protein, 14 aa, Artificial Sequence
  Synthetic Polypeptide
SGEGSFQPSQENPQ

SEQ ID NO: 55 - protein, 14 aa, Artificial Sequence
  Synthetic Polypeptide
GQQGYYPTSPQQSG

SEQ ID NO: 56 - protein, 14 aa, Artificial Sequence
  Synthetic Polypeptide
PEQPEQPFPEQPQQ

SEQ ID NO: 57 - protein, 14 aa, Artificial Sequence
  Synthetic Polypeptide
QPPFSEQEQPVLPQ

SEQ ID NO: 58 - protein, 14 aa, Artificial Sequence
  Synthetic Polypeptide
QPYPQPELPYPQPQ

SEQ ID NO: 59 - protein, 14 aa, Artificial Sequence
  Synthetic Polypeptide
QPFPQPELPYPYPQ

SEQ ID NO: 60 - protein, 15 aa, Artificial Sequence
  Synthetic Polypeptide
PQEQPFPEQPIPEQP

SEQ ID NO: 61 - protein, 15 aa, Artificial Sequence
  Synthetic Polypeptide
QPQPYPEQPQPFPQQ

SEQ ID NO: 62 - protein, 10 aa, Artificial Sequence
  Synthetic Polypeptide
PFPQPEQPIP

SEQ ID NO: 63 - protein, 11 aa, Artificial Sequence
  Synthetic Polypeptide
PFPQPEQPTPI

SEQ ID NO: 64 - protein, 11 aa, Artificial Sequence
  Synthetic Polypeptide
PFPEQPEQIIP

SEQ ID NO: 65 - protein, 12 aa, Artificial Sequence
  Synthetic Polypeptide
EQPEQPFPEQPQ

SEQ ID NO: 66 - DNA, 1227 bp, Homo sapiens
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 67 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 68 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 69 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 70 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 71 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 72 - protein, 4 aa, Artificial Sequence
  Synthetic Polypeptide
PELP

SEQ ID NO: 73 - protein, 5 aa, Artificial Sequence
  Synthetic Polypeptide
PELPY

SEQ ID NO: 74 - protein, 7 aa, Artificial Sequence
  Synthetic Polypeptide
QPELPYP

SEQ ID NO: 75 - protein, 7 aa, Artificial Sequence
  Synthetic Polypeptide
PQPELPY

SEQ ID NO: 76 - protein, 7 aa, Artificial Sequence
  Synthetic Polypeptide
FPQPELP

SEQ ID NO: 77 - protein, 7 aa, Artificial Sequence
  Synthetic Polypeptide
PELPYPQ

SEQ ID NO: 78 - protein, 9 aa, Artificial Sequence
  Synthetic Polypeptide
FPQPELPYP

SEQ ID NO: 79 - protein, 9 aa, Artificial Sequence
  Synthetic Polypeptide
FRPEQPYPQ

SEQ ID NO: 80 - protein, 9 aa, Artificial Sequence
  Synthetic Polypeptide
PQQSFPEQQ

SEQ ID NO: 81 - protein, 9 aa, Artificial Sequence
  Synthetic Polypeptide
IQPEQPAQL

SEQ ID NO: 82 - protein, 9 aa, Artificial Sequence
  Synthetic Polypeptide
QQPEQPYPQ

SEQ ID NO: 83 - protein, 9 aa, Artificial Sequence
  Synthetic Polypeptide
SQPEQEFPQ

SEQ ID NO: 84 - protein, 9 aa, Artificial Sequence
  Synthetic Polypeptide
PQPEQEFPQ

SEQ ID NO: 85 - protein, 9 aa, Artificial Sequence
  Synthetic Polypeptide
QQPEQPFPQ

SEQ ID NO: 86 - protein, 9 aa, Artificial Sequence
  Synthetic Polypeptide
PQPEQPFCQ

SEQ ID NO: 87 - protein, 9 aa, Artificial Sequence
  Synthetic Polypeptide
QQPFPEQPQ

SEQ ID NO: 88 - protein, 9 aa, Artificial Sequence
  Synthetic Polypeptide
FSQQQESPF

SEQ ID NO: 89 - protein, 9 aa, Artificial Sequence
  Synthetic Polypeptide
PQPEQPFPQ

SEQ ID NO: 90 - protein, 9 aa, Artificial Sequence
  Synthetic Polypeptide
PQPEQPFPQ

SEQ ID NO: 91 - protein, 9 aa, Artificial Sequence
  Synthetic Polypeptide
PYPEQEEPF

SEQ ID NO: 92 - protein, 9 aa, Artificial Sequence
  Synthetic Polypeptide
PYPEQEQPF

SEQ ID NO: 93 - protein, 9 aa, Artificial Sequence
  Synthetic Polypeptide
EQPQQPFPQ

SEQ ID NO: 94 - protein, 9 aa, Artificial Sequence
  Synthetic Polypeptide
EQPQQPYPE

SEQ ID NO: 95 - protein, 10 aa, Artificial Sequence
  Synthetic Polypeptide
QQGYYPTSPQ

SEQ ID NO: 96 - protein, 9 aa, Artificial Sequence
  Synthetic Polypeptide
PQQSFPEQE

SEQ ID NO: 97 - protein, 9 aa, Artificial Sequence
  Synthetic Polypeptide
PFPQPQLPY

SEQ ID NO: 98 - protein, 9 aa, Artificial Sequence
  Synthetic Polypeptide
PFPQPDLPY

SEQ ID NO: 99 - protein, 9 aa, Artificial Sequence
  Synthetic Polypeptide
PQPDLPYPQ

SEQ ID NO: 100 - protein, 9 aa, Artificial Sequence
  Synthetic Polypeptide
PFPQPDQPF

SEQ ID NO: 101 - protein, 9 aa, Artificial Sequence
  Synthetic Polypeptide
PQPDQPFPW

SEQ ID NO: 102 - protein, 9 aa, Artificial Sequence
  Synthetic Polypeptide
PIPDQPQPY

SEQ ID NO: 103 - protein, 15 aa, Artificial Sequence
  Synthetic Polypeptide
LQPFPQPDLPYPQPQ

SEQ ID NO: 104 - protein, 14 aa, Artificial Sequence
  Synthetic Polypeptide
QPFPQPDQPFPWQP

SEQ ID NO: 105 - protein, 15 aa, Artificial Sequence
  Synthetic Polypeptide
PQQPIPDQPQPYPQQ

SEQ ID NO: 106 - protein, 10 aa, Artificial Sequence
  Synthetic Polypeptide
PFPQPDQPIP

SEQ ID NO: 107 - protein, 9 aa, Artificial Sequence
  Synthetic Polypeptide
DQPIPVQPD

SEQ ID NO: 108 - protein, 11 aa, Artificial Sequence
  Synthetic Polypeptide
PFPQPDQPTPI

SEQ ID NO: 109 - protein, 9 aa, Artificial Sequence
  Synthetic Polypeptide
DQPTPIQPD

SEQ ID NO: 110 - protein, 9 aa, Artificial Sequence
  Synthetic Polypeptide
PQPDQPFPL

SEQ ID NO: 111 - protein, 9 aa, Artificial Sequence
  Synthetic Polypeptide
DQPFPLQPD

SEQ ID NO: 112 - protein, 9 aa, Artificial Sequence
  Synthetic Polypeptide
PFPQPDQPF

SEQ ID NO: 113 - protein, 9 aa, Artificial Sequence
  Synthetic Polypeptide
PQPDQPFSQ

SEQ ID NO: 114 - protein, 9 aa, Artificial Sequence
  Synthetic Polypeptide
PYPDQPQPF

SEQ ID NO: 115 - protein, 11 aa, Artificial Sequence
  Synthetic Polypeptide
PFPDQPDQIIP

SEQ ID NO: 116 - protein, 9 aa, Artificial Sequence
  Synthetic Polypeptide
DGSFQPSQD

SEQ ID NO: 117 - protein, 12 aa, Artificial Sequence
  Synthetic Polypeptide
DQPDQPFPDQPQ

SEQ ID NO: 118 - protein, 9 aa, Artificial Sequence
  Synthetic Polypeptide
PFSDQDQPV

SEQ ID NO: 119 - protein, 9 aa, Artificial Sequence
  Synthetic Polypeptide
DQPFPDQPI

SEQ ID NO: 120 - protein, 9 aa, Artificial Sequence
  Synthetic Polypeptide
PIPDQPQPY

SEQ ID NO: 121 - protein, 9 aa, Artificial Sequence
  Synthetic Polypeptide
PQPDLPYPQ

SEQ ID NO: 122 - protein, 9 aa, Artificial Sequence
  Synthetic Polypeptide
PYPQPDLPY

SEQ ID NO: 123 - protein, 9 aa, Artificial Sequence
  Synthetic Polypeptide
PQPDLPYPY

SEQ ID NO: 124 - protein, 9 aa, Artificial Sequence
  Synthetic Polypeptide
PQPDQPFPW

SEQ ID NO: 125 - protein, 9 aa, Artificial Sequence
  Synthetic Polypeptide
PIPDQPQPY

SEQ ID NO: 126 - protein, 9 aa, Artificial Sequence
  Synthetic Polypeptide
PFPQPDQPI

SEQ ID NO: 127 - protein, 9 aa, Artificial Sequence
  Synthetic Polypeptide
PQPDQPIPV

SEQ ID NO: 128 - protein, 9 aa, Artificial Sequence
  Synthetic Polypeptide
DQPIPVQPE

SEQ ID NO: 129 - protein, 9 aa, Artificial Sequence
  Synthetic Polypeptide
PFPQPDQPT

SEQ ID NO: 130 - protein, 9 aa, Artificial Sequence
  Synthetic Polypeptide
PQPDQPTPI

SEQ ID NO: 131 - protein, 9 aa, Artificial Sequence
  Synthetic Polypeptide
DQPTPIQPD

SEQ ID NO: 132 - protein, 9 aa, Artificial Sequence
  Synthetic Polypeptide
PQPDQPFPL

SEQ ID NO: 133 - protein, 9 aa, Artificial Sequence
  Synthetic Polypeptide
DQPFPLQPD

SEQ ID NO: 134 - protein, 9 aa, Artificial Sequence
  Synthetic Polypeptide
PYPDQPQPF

SEQ ID NO: 135 - protein, 9 aa, Artificial Sequence
  Synthetic Polypeptide
DGSFQPSQD

SEQ ID NO: 136 - protein, 9 aa, Artificial Sequence
  Synthetic Polypeptide
DQPQQPFPD

SEQ ID NO: 137 - protein, 9 aa, Artificial Sequence
  Synthetic Polypeptide
DQPDQPFPQ

SEQ ID NO: 138 - protein, 9 aa, Artificial Sequence
  Synthetic Polypeptide
DQPFPDQPQ

SEQ ID NO: 139 - protein, 9 aa, Artificial Sequence
  Synthetic Polypeptide
PFPDQPDQI

SEQ ID NO: 140 - protein, 9 aa, Artificial Sequence
  Synthetic Polypeptide
PFSDQDQPV

SEQ ID NO: 141 - protein, 9 aa, Artificial Sequence
  Synthetic Polypeptide
DQPFPDQPI

SEQ ID NO: 142 - protein, 9 aa, Artificial Sequence
  Synthetic Polypeptide
PFPDQPIPD

SEQ ID NO: 143 - protein, 9 aa, Artificial Sequence
  Synthetic Polypeptide
PYPQPDLPY

SEQ ID NO: 144 - protein, 9 aa, Artificial Sequence
  Synthetic Polypeptide
PQPYPDQPQ

SEQ ID NO: 145 - protein, 9 aa, Artificial Sequence
  Synthetic Polypeptide
PQPDQPFSQ

SEQ ID NO: 146 - protein, 9 aa, Artificial Sequence
  Synthetic Polypeptide
DQPIPDQPQ

SEQ ID NO: 147 - protein, 4 aa, Artificial Sequence
  Synthetic Polypeptide
PQLP

SEQ ID NO: 148 - protein, 5 aa, Artificial Sequence
  Synthetic Polypeptide
PQLPY

SEQ ID NO: 149 - protein, 7 aa, Artificial Sequence
  Synthetic Polypeptide
QPQLPYP

SEQ ID NO: 150 - protein, 7 aa, Artificial Sequence
  Synthetic Polypeptide
PQPQLPY

SEQ ID NO: 151 - protein, 7 aa, Artificial Sequence
  Synthetic Polypeptide
FPQPQLP

SEQ ID NO: 152 - protein, 7 aa, Artificial Sequence
  Synthetic Polypeptide
PQLPYPQ

SEQ ID NO: 153 - protein, 9 aa, Artificial Sequence
  Synthetic Polypeptide
FPQPQLPYP

SEQ ID NO: 154 - protein, 9 aa, Artificial Sequence
  Synthetic Polypeptide
PYPQPQLPY

SEQ ID NO: 155 - protein, 9 aa, Artificial Sequence
  Synthetic Polypeptide
PQPQLPYPQ

SEQ ID NO: 156 - protein, 9 aa, Artificial Sequence
  Synthetic Polypeptide
PFPQPQQPF

SEQ ID NO: 157 - protein, 9 aa, Artificial Sequence
  Synthetic Polypeptide
PQPQQPFPW

SEQ ID NO: 158 - protein, 9 aa, Artificial Sequence
  Synthetic Polypeptide
PIPQQPQPY

SEQ ID NO: 159 - protein, 15 aa, Artificial Sequence
  Synthetic Polypeptide
LQPFPQPQLPYPQPQ

SEQ ID NO: 160 - protein, 14 aa, Artificial Sequence
  Synthetic Polypeptide
QPFPQPQQPFPWQP

SEQ ID NO: 161 - protein, 15 aa, Artificial Sequence
  Synthetic Polypeptide
PEQPIPQQPQPYPQQ

SEQ ID NO: 162 - protein, 9 aa, Artificial Sequence
  Synthetic Polypeptide
PQPQLPYPQ

SEQ ID NO: 163 - protein, 9 aa, Artificial Sequence
  Synthetic Polypeptide
FRPQQPYPQ

SEQ ID NO: 164 - protein, 9 aa, Artificial Sequence
  Synthetic Polypeptide
PQQSFPQQQ

SEQ ID NO: 165 - protein, 9 aa, Artificial Sequence
  Synthetic Polypeptide
IQPQQPAQL

SEQ ID NO: 166 - protein, 9 aa, Artificial Sequence
  Synthetic Polypeptide
QQPQQPYPQ

SEQ ID NO: 167 - protein, 9 aa, Artificial Sequence
  Synthetic Polypeptide
SQPQQQFPQ

SEQ ID NO: 168 - protein, 9 aa, Artificial Sequence
  Synthetic Polypeptide
PQPQQQFPQ

SEQ ID NO: 169 - protein, 9 aa, Artificial Sequence
  Synthetic Polypeptide
QQPQQPFPQ

SEQ ID NO: 170 - protein, 9 aa, Artificial Sequence
  Synthetic Polypeptide
PQPQQPFCQ

SEQ ID NO: 171 - protein, 9 aa, Artificial Sequence
  Synthetic Polypeptide
QQPFPQQPQ

SEQ ID NO: 172 - protein, 9 aa, Artificial Sequence
  Synthetic Polypeptide
PFPQPQQPF

SEQ ID NO: 173 - protein, 9 aa, Artificial Sequence
  Synthetic Polypeptide
PQPQQPFPW

SEQ ID NO: 174 - protein, 9 aa, Artificial Sequence
  Synthetic Polypeptide
PFSQQQQPV

SEQ ID NO: 175 - protein, 9 aa, Artificial Sequence
  Synthetic Polypeptide
FSQQQQSPF

SEQ ID NO: 176 - protein, 9 aa, Artificial Sequence
  Synthetic Polypeptide
PQPQQPFPQ

SEQ ID NO: 177 - protein, 9 aa, Artificial Sequence
  Synthetic Polypeptide
PIPQQPQPY

SEQ ID NO: 178 - protein, 9 aa, Artificial Sequence
  Synthetic Polypeptide
PFPQPQQPF

SEQ ID NO: 179 - protein, 9 aa, Artificial Sequence
  Synthetic Polypeptide
PQPQQPFPQ

SEQ ID NO: 180 - protein, 9 aa, Artificial Sequence
  Synthetic Polypeptide
PYPEQQEPF

SEQ ID NO: 181 - protein, 9 aa, Artificial Sequence
  Synthetic Polypeptide
PYPEQQQPF

SEQ ID NO: 182 - protein, 9 aa, Artificial Sequence
  Synthetic Polypeptide
PFSQQQQPV

SEQ ID NO: 183 - protein, 9 aa, Artificial Sequence
  Synthetic Polypeptide
QGSFQPSQQ

SEQ ID NO: 184 - protein, 9 aa, Artificial Sequence
  Synthetic Polypeptide
QQPQQPFPQ

SEQ ID NO: 185 - protein, 9 aa, Artificial Sequence
  Synthetic Polypeptide
QQPQQPYPQ

SEQ ID NO: 186 - protein, 9 aa, Artificial Sequence
  Synthetic Polypeptide
QGSFQPSQQ

SEQ ID NO: 187 - protein, 9 aa, Artificial Sequence
  Synthetic Polypeptide
PQQSFPQQQ

SEQ ID NO: 188 - protein, 14 aa, Artificial Sequence
  Synthetic Polypeptide
QPFPQPQQPFPWQP

SEQ ID NO: 189 - protein, 15 aa, Artificial Sequence
  Synthetic Polypeptide
PQQPIPQQPQPYPQQ

SEQ ID NO: 190 - protein, 15 aa, Artificial Sequence
  Synthetic Polypeptide
LQPFPQPQLPYPQPQ

SEQ ID NO: 191 - protein, 14 aa, Artificial Sequence
  Synthetic Polypeptide
QPFPQPQQPFPWQP

SEQ ID NO: 192 - protein, 15 aa, Artificial Sequence
  Synthetic Polypeptide
PQQPIPQQPQPYPQQ

SEQ ID NO: 193 - protein, 17 aa, Artificial Sequence
  Synthetic Polypeptide
QPFPQPQQPIPVQPQQS

SEQ ID NO: 194 - protein, 17 aa, Artificial Sequence
  Synthetic Polypeptide
QPFPQPQQPTPIQPQQP

SEQ ID NO: 195 - protein, 17 aa, Artificial Sequence
  Synthetic Polypeptide
QPFPQPQQPFPLQPQQP

SEQ ID NO: 196 - protein, 13 aa, Artificial Sequence
  Synthetic Polypeptide
QPFPQPQQPFSQQ

SEQ ID NO: 197 - protein, 14 aa, Artificial Sequence
  Synthetic Polypeptide
PQPYPQQPQPFPQQ

SEQ ID NO: 198 - protein, 15 aa, Artificial Sequence
  Synthetic Polypeptide
QPFPEQPQQIIPQQP

SEQ ID NO: 199 - protein, 14 aa, Artificial Sequence
  Synthetic Polypeptide
SGEGSFQPSQQNPQ

SEQ ID NO: 200 - protein, 14 aa, Artificial Sequence
  Synthetic Polypeptide
PQQPQQPFPQQPQQ

SEQ ID NO: 201 - protein, 14 aa, Artificial Sequence
  Synthetic Polypeptide
QPPFSQQQQPVLPQ

SEQ ID NO: 202 - protein, 18 aa, Artificial Sequence
  Synthetic Polypeptide
PQQPFPQQPIPQQPQPYP

SEQ ID NO: 203 - protein, 14 aa, Artificial Sequence
  Synthetic Polypeptide
QPYPQPQLPYPQPQ

SEQ ID NO: 204 - protein, 14 aa, Artificial Sequence
  Synthetic Polypeptide
QPFPQPQLPYPYPQ

SEQ ID NO: 205 - protein, 9 aa, Artificial Sequence
  Synthetic Polypeptide
PQPQLPYPQ

SEQ ID NO: 206 - protein, 9 aa, Artificial Sequence
  Synthetic Polypeptide
PFPQPQQPF

SEQ ID NO: 207 - protein, 9 aa, Artificial Sequence
  Synthetic Polypeptide
PQPQQPFPW

SEQ ID NO: 208 - protein, 9 aa, Artificial Sequence
  Synthetic Polypeptide
QQPIPQQPQ

SEQ ID NO: 209 - protein, 9 aa, Artificial Sequence
  Synthetic Polypeptide
PIPQQPQPY

SEQ ID NO: 210 - protein, 9 aa, Artificial Sequence
  Synthetic Polypeptide
PFPQPQQPI

SEQ ID NO: 211 - protein, 9 aa, Artificial Sequence
  Synthetic Polypeptide
PQPQQPIPV

SEQ ID NO: 212 - protein, 9 aa, Artificial Sequence
  Synthetic Polypeptide
QQPIPVQPQ

SEQ ID NO: 213 - protein, 9 aa, Artificial Sequence
  Synthetic Polypeptide
PFPQPQQPT

SEQ ID NO: 214 - protein, 9 aa, Artificial Sequence
  Synthetic Polypeptide
PQPQQPTPI

SEQ ID NO: 215 - protein, 9 aa, Artificial Sequence
  Synthetic Polypeptide
QQPTPIQPQ

SEQ ID NO: 216 - protein, 9 aa, Artificial Sequence
  Synthetic Polypeptide
PQPQQPFPL

SEQ ID NO: 217 - protein, 9 aa, Artificial Sequence
  Synthetic Polypeptide
QQPFPLQPQ

SEQ ID NO: 218 - protein, 9 aa, Artificial Sequence
  Synthetic Polypeptide
PQPQQPFSQ

SEQ ID NO: 219 - protein, 9 aa, Artificial Sequence
  Synthetic Polypeptide
PYPQQPQPF

SEQ ID NO: 220 - protein, 9 aa, Artificial Sequence
  Synthetic Polypeptide
QGSFQPSQQ

SEQ ID NO: 221 - protein, 9 aa, Artificial Sequence
  Synthetic Polypeptide
QQPQQPFPQ

SEQ ID NO: 222 - protein, 10 aa, Artificial Sequence
  Synthetic Polypeptide
QQGYYPTSPQ

SEQ ID NO: 223 - protein, 9 aa, Artificial Sequence
  Synthetic Polypeptide
QQPFPQQPQ

SEQ ID NO: 224 - protein, 9 aa, Artificial Sequence
  Synthetic Polypeptide
PFPQQPQQI

SEQ ID NO: 225 - protein, 9 aa, Artificial Sequence
  Synthetic Polypeptide
PFSQQQQPV

SEQ ID NO: 226 - protein, 9 aa, Artificial Sequence
  Synthetic Polypeptide
QQPFPQQPI

SEQ ID NO: 227 - protein, 9 aa, Artificial Sequence
  Synthetic Polypeptide
PFPQQPIPQ

SEQ ID NO: 228 - protein, 9 aa, Artificial Sequence
  Synthetic Polypeptide
PYPQPQLPY

SEQ ID NO: 229 - protein, 9 aa, Artificial Sequence
  Synthetic Polypeptide
PQPQLPYPY

SEQ ID NO: 230 - protein, 9 aa, Artificial Sequence
  Synthetic Polypeptide
PQPYPQQPQ

SEQ ID NO: 231 - protein, 17 aa, Artificial Sequence
  Synthetic Polypeptide
QLQPFPQPELPYPQPQS

SEQ ID NO: 232 - protein, 16 aa, Artificial Sequence
  Synthetic Polypeptide
XLQPFPQPELPYPQPQ

SEQ ID NO: 233 - protein, 15 aa, Artificial Sequence
  Synthetic Polypeptide
XQPFPQPEQPFPWQP

SEQ ID NO: 234 - protein, 16 aa, Artificial Sequence
  Synthetic Polypeptide
XPEQPIPEQPQPYPQQ

SEQ ID NO: 235 - protein, 16 aa, Artificial Sequence
  Synthetic Polypeptide
XLQPFPQPELPYPQPQ

SEQ ID NO: 236 - protein, 15 aa, Artificial Sequence
  Synthetic Polypeptide
XQPFPQPEQPFPWQP

SEQ ID NO: 237 - protein, 16 aa, Artificial Sequence
  Synthetic Polypeptide
XPEQPIPEQPQPYPQQ

SEQ ID NO: 238 - protein, 16 aa, Artificial Sequence
  Synthetic Polypeptide
XLQPFPQPELPYPQPQ

SEQ ID NO: 239 - protein, 15 aa, Artificial Sequence
  Synthetic Polypeptide
XQPFPQPEQPFPWQP

SEQ ID NO: 240 - protein, 16 aa, Artificial Sequence
  Synthetic Polypeptide
XPEQPIPEQPQPYPQQ

SEQ ID NO: 241 - protein, 18 aa, Artificial Sequence
  Synthetic Polypeptide
XQPFPQPEQPIPVQPEQS

SEQ ID NO: 242 - protein, 18 aa, Artificial Sequence
  Synthetic Polypeptide
XQPFPQPEQPTPIQPEQP

SEQ ID NO: 243 - protein, 18 aa, Artificial Sequence
  Synthetic Polypeptide
XQPFPQPEQPFPLQPEQP

SEQ ID NO: 244 - protein, 14 aa, Artificial Sequence
  Synthetic Polypeptide
XQPFPQPEQPFSQQ

SEQ ID NO: 245 - protein, 15 aa, Artificial Sequence
  Synthetic Polypeptide
XPQPYPEQPQPFPQQ

SEQ ID NO: 246 - protein, 16 aa, Artificial Sequence
  Synthetic Polypeptide
XQPFPEQPEQIIPQQP

SEQ ID NO: 247 - protein, 15 aa, Artificial Sequence
  Synthetic Polypeptide
XSGEGSFQPSQENPQ

SEQ ID NO: 248 - protein, 15 aa, Artificial Sequence
  Synthetic Polypeptide
XGQQGYYPTSPQQSG

SEQ ID NO: 249 - protein, 15 aa, Artificial Sequence
  Synthetic Polypeptide
XPEQPEQPFPEQPQQ

SEQ ID NO: 250 - protein, 15 aa, Artificial Sequence
  Synthetic Polypeptide
XQPPFSEQEQPVLPQ

SEQ ID NO: 251 - protein, 15 aa, Artificial Sequence
  Synthetic Polypeptide
XQPFPQPEQPFPWQP

SEQ ID NO: 252 - protein, 18 aa, Artificial Sequence
  Synthetic Polypeptide
XQPFPQPEQPIPVQPEQS

SEQ ID NO: 253 - protein, 18 aa, Artificial Sequence
  Synthetic Polypeptide
XQPFPQPEQPTPIQPEQP

SEQ ID NO: 254 - protein, 18 aa, Artificial Sequence
  Synthetic Polypeptide
XQPFPQPEQPFPLQPEQP

SEQ ID NO: 255 - protein, 14 aa, Artificial Sequence
  Synthetic Polypeptide
XQPFPQPEQPFSQQ

SEQ ID NO: 256 - protein, 15 aa, Artificial Sequence
  Synthetic Polypeptide
XPQPYPEQPQPFPQQ

SEQ ID NO: 257 - protein, 16 aa, Artificial Sequence
  Synthetic Polypeptide
XQPFPEQPEQIIPQQP

SEQ ID NO: 258 - protein, 15 aa, Artificial Sequence
  Synthetic Polypeptide
XSGEGSFQPSQENPQ

SEQ ID NO: 259 - protein, 15 aa, Artificial Sequence
  Synthetic Polypeptide
XGQQGYYPTSPQQSG

SEQ ID NO: 260 - protein, 15 aa, Artificial Sequence
  Synthetic Polypeptide
XPEQPEQPFPEQPQQ

SEQ ID NO: 261 - protein, 15 aa, Artificial Sequence
  Synthetic Polypeptide
XQPPFSEQEQPVLPQ

SEQ ID NO: 262 - protein, 19 aa, Artificial Sequence
  Synthetic Polypeptide
XPEQPFPEQPIPEQPQPYP

SEQ ID NO: 263 - protein, 15 aa, Artificial Sequence
  Synthetic Polypeptide
XQPYPQPELPYPQPQ

SEQ ID NO: 264 - protein, 15 aa, Artificial Sequence
  Synthetic Polypeptide
XQPFPQPELPYPYPQ

SEQ ID NO: 265 - protein, 16 aa, Artificial Sequence
  Synthetic Polypeptide
XLQPFPQPELPYPQPQ

SEQ ID NO: 266 - protein, 15 aa, Artificial Sequence
  Synthetic Polypeptide
XQPFPQPEQPFPWQP

SEQ ID NO: 267 - protein, 16 aa, Artificial Sequence
  Synthetic Polypeptide
XPEQPIPEQPQPYPQQ

SEQ ID NO: 268 - protein, 18 aa, Artificial Sequence
  Synthetic Polypeptide
XQPFPQPEQPIPVQPEQS

SEQ ID NO: 269 - protein, 18 aa, Artificial Sequence
  Synthetic Polypeptide
XQPFPQPEQPTPIQPEQP

SEQ ID NO: 270 - protein, 18 aa, Artificial Sequence
  Synthetic Polypeptide
XQPFPQPEQPFPLQPEQP

SEQ ID NO: 271 - protein, 14 aa, Artificial Sequence
  Synthetic Polypeptide
XQPFPQPEQPFSQQ

SEQ ID NO: 272 - protein, 15 aa, Artificial Sequence
  Synthetic Polypeptide
XPQPYPEQPQPFPQQ

SEQ ID NO: 273 - protein, 15 aa, Artificial Sequence
  Synthetic Polypeptide
XSGEGSFQPSQENPQ

SEQ ID NO: 274 - protein, 15 aa, Artificial Sequence
  Synthetic Polypeptide
XGQQGYYPTSPQQSG

SEQ ID NO: 275 - protein, 15 aa, Artificial Sequence
  Synthetic Polypeptide
XPEQPEQPFPEQPQQ

SEQ ID NO: 276 - protein, 16 aa, Artificial Sequence
  Synthetic Polypeptide
XQPFPEQPEQIIPQQP

SEQ ID NO: 277 - protein, 15 aa, Artificial Sequence
  Synthetic Polypeptide
XQPPFSEQEQPVLPQ

SEQ ID NO: 278 - protein, 19 aa, Artificial Sequence
  Synthetic Polypeptide
XPEQPFPEQPIPEQPQPYP

SEQ ID NO: 279 - protein, 15 aa, Artificial Sequence
  Synthetic Polypeptide
XQPYPQPELPYPQPQ

SEQ ID NO: 280 - protein, 15 aa, Artificial Sequence
  Synthetic Polypeptide
XQPFPQPELPYPYPQ

SEQ ID NO: 281 - protein, 16 aa, Artificial Sequence
  Synthetic Polypeptide
XPQEQPFPEQPIPEQP

SEQ ID NO: 282 - protein, 16 aa, Artificial Sequence
  Synthetic Polypeptide
XQPQPYPEQPQPFPQQ

SEQ ID NO: 283 - protein, 19 aa, Artificial Sequence
  Synthetic Polypeptide
ZPEQPFPEQPIPEQPQPYP

SEQ ID NO: 284 - protein, 5 aa, Artificial Sequence
  Synthetic Polypeptide
EQPFP

SEQ ID NO: 285 - protein, 12 aa, Artificial Sequence
  Synthetic Polypeptide
QQQPFPQQPIPQ

SEQ ID NO: 286 - protein, 13 aa, Artificial Sequence
  Synthetic Polypeptide
XPFPQPELPYPQP

SEQ ID NO: 287 - protein, 13 aa, Artificial Sequence
  Synthetic Polypeptide
XPFPQPEQPFPWQ

SEQ ID NO: 288 - protein, 13 aa, Artificial Sequence
  Synthetic Polypeptide
XEQPIPEQPQPYP

SEQ ID NO: 289 - protein, 13 aa, Artificial Sequence
  Synthetic Polypeptide
XPFPQPEQPIPVQ

SEQ ID NO: 290 - protein, 13 aa, Artificial Sequence
  Synthetic Polypeptide
XPEQPIPVQPEQS

SEQ ID NO: 291 - protein, 13 aa, Artificial Sequence
  Synthetic Polypeptide
XPFPQPEQPTPIQ

SEQ ID NO: 292 - protein, 13 aa, Artificial Sequence
  Synthetic Polypeptide
XPEQPTPIQPEQP

SEQ ID NO: 293 - protein, 13 aa, Artificial Sequence
  Synthetic Polypeptide
XPFPQPEQPFPLQ

SEQ ID NO: 294 - protein, 13 aa, Artificial Sequence
  Synthetic Polypeptide
XPEQPFPLQPEQP

SEQ ID NO: 295 - protein, 13 aa, Artificial Sequence
  Synthetic Polypeptide
XGEGSFQPSQENP

SEQ ID NO: 296 - protein, 13 aa, Artificial Sequence
  Synthetic Polypeptide
XQQGYYPTSPQQS

SEQ ID NO: 297 - protein, 13 aa, Artificial Sequence
  Synthetic Polypeptide
XPEQPEQPFPEQP

SEQ ID NO: 298 - protein, 13 aa, Artificial Sequence
  Synthetic Polypeptide
XPPFSEQEQPVLP

SEQ ID NO: 299 - protein, 13 aa, Artificial Sequence
  Synthetic Polypeptide
XPYPQPELPYPQP

SEQ ID NO: 300 - protein, 13 aa, Artificial Sequence
  Synthetic Polypeptide
XEQPFPEQPIPEQ

SEQ ID NO: 301 - protein, 13 aa, Artificial Sequence
  Synthetic Polypeptide
XPQPYPEQPQPFP

(19) United States
(12) Patent Application Publication
(10) Pub. No.: US 2017/0045513 A1
(43) Pub. Date: Feb. 16, 2017

(54) METHODS OF DIAGNOSING CELIAC DISEASE USING IP-10

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 15/306,189

(22) PCT Filed: Apr. 24, 2015
(86) PCT No.: PCT/US2015/027488

§ 371 Date: Oct. 24, 2016

Related U.S. Application Data
(60) Provisional application No. 62/115,925, filed on Feb. 13, 2015.
(60) Provisional application No. 62/115,897, filed on Feb. 13, 2015.
(60) Provisional application No. 62/116,052, filed on Feb. 13, 2015.
(60) Provisional application No. 62/116,002, filed on Feb. 13, 2015.
(60) Provisional application No. 62/115,963, filed on Feb. 13, 2015.
(60) Provisional application No. 62/116,027, filed on Feb. 13, 2015.
(60) Provisional application No. 62/082,832, filed on Nov. 21, 2014.
(60) Provisional application No. 62/057,152, filed on Sep. 29, 2014.
(60) Provisional application No. 62/057,163, filed on Sep. 29, 2014.
(60) Provisional application No. 62/043,386, filed on Aug. 28, 2014.
(60) Provisional application No. 62/043,390, filed on Aug. 28, 2014.
(60) Provisional application No. 62/043,395, filed on Aug. 28, 2014.
(60) Provisional application No. 62/014,666, filed on Jun. 19, 2014.
(60) Provisional application No. 62/014,676, filed on Jun. 19, 2014.
(60) Provisional application No. 62/014,681, filed on Jun. 19, 2014.
(60) Provisional application No. 62/014,373, filed on Jun. 19, 2014.
(60) Provisional application No. 62/014,401, filed on Jun. 19, 2014.
(60) Provisional application No. 62/011,794, filed on Jun. 13, 2014.
(60) Provisional application No. 62/011,493, filed on Jun. 12, 2014.
(60) Provisional application No. 62/011,540, filed on Jun. 12, 2014.
(60) Provisional application No. 62/011,508, filed on Jun. 12, 2014.
(60) Provisional application No. 62/011,561, filed on Jun. 12, 2014.
(60) Provisional application No. 62/011,566, filed on Jun. 12, 2014.
(60) Provisional application No. 62/009,090, filed on Jun. 6, 2014.
(60) Provisional application No. 62/009,146, filed on Jun. 6, 2014.
(60) Provisional application No. 61/984,043, filed on Apr. 25, 2014.
(60) Provisional application No. 61/983,989, filed on Apr. 24, 2014.
(60) Provisional application No. 61/983,993, filed on Apr. 24, 2014.
(60) Provisional application No. 61/983,981, filed on Apr. 24, 2014.
(60) Provisional application No. 61/984,028, filed on Apr. 24, 2014.

(51) Int. Cl. G01N 33/564; A61K 38/16; G01N 33/68

(52) U.S. Cl. CPC G01N 33/564; G01N 33/6863; A61K 38/168; G01N 2333/522; G01N 2333/415; G01N 2800/06; G01N 2800/50

(57) ABSTRACT

Provided herein are methods of identifying a subject having or at risk for having Celiac disease by determining a level of IP-10 in a sample from a subject.

### RELATED APPLICATIONS

This application claims the benefit under 35 U.S.C. §119(e) of U.S. provisional application No. 61/983,981, filed Apr. 24, 2014, U.S. provisional application No. 62/011,561, filed Jun. 12, 2014, U.S. provisional application No. 62/014,676, filed Jun. 19, 2014, U.S. provisional application No. 62/057,152, filed Sep. 29, 2014, U.S. provisional application No. 62/115,925, filed Feb. 13, 2015, U.S. provisional application No. 61/984,028, filed Apr. 24, 2014, U.S. provisional application No. 61/984,043, filed Apr. 25, 2014, U.S. provisional application No. 62/011,566, filed Jun. 12, 2014, U.S. provisional application No. 62/014,681, filed Jun. 19, 2014, U.S. provisional application No. 62/057,163, filed Sep. 29, 2014, U.S. provisional application No. 62/115,897, filed Feb. 13, 2015, U.S. provisional application No. 61/983,989, filed Apr. 24, 2014, U.S. provisional application No. 62/014,666, filed Jun. 19, 2014, U.S. provisional application No. 62/009,146, filed Jun. 6, 2014, U.S. provisional application No. 62/043,386, filed Aug. 28, 2014, U.S. provisional application No. 62/115,963, filed Feb. 13, 2015, U.S. provisional application No. 61/983,993, filed Apr. 24, 2014, U.S. provisional application No. 62/011,508, filed Jun. 12, 2014, U.S. provisional application No. 62/116,052, filed Feb. 13, 2015, U.S. provisional application No. 62/043,395, filed Aug. 28, 2014, U.S. provisional application No. 62/082,832, filed Nov. 21, 2014, U.S. provisional application No. 62/009,090, filed Jun. 6, 2014, U.S. provisional application No. 62/014,373, filed Jun. 19, 2014, U.S. provisional application No. 62/043,390, filed Aug. 28, 2014, U.S. provisional application No. 62/116,002, filed Feb. 13, 2015, U.S. provisional application No. 62/011,493, filed Jun. 12, 2014, U.S. provisional application No. 62/011,794, filed Jun. 13, 2014, U.S. provisional application No. 62/014,401, filed Jun. 19, 2014, U.S. provisional application No. 62/116,027, filed Feb. 13, 2015, and U.S. provisional application No. 62/011,540, filed Jun. 12, 2014, the contents of each of which are incorporated by reference herein in their entirety.

### BACKGROUND

Celiac disease is an autoimmune disorder of the small intestine that occurs in people of all ages. Celiac disease causes damage to the villi of the small intestine due to an inappropriate immune response to gluten peptides, leading to malabsorption and an increased risk of intestinal cancer. Identifying subjects with Celiac disease is important for ensuring that such Celiac disease subjects receive proper treatment.

### SUMMARY

As described herein, it has been found that IFN-γ inducible protein-10 (IP-10) levels are highly elevated in blood samples obtained from subjects with Celiac disease, even if the subject has not undergone an oral gluten challenge prior to the sample being obtained from the subject.

Accordingly, aspects of the disclosure relate to methods of identifying (e.g., diagnosing) a subject as having or at risk of having Celiac disease by determining a level of IP-10 in a sample from the subject.

In some aspects, the disclosure relates to a method of identifying a subject having or at risk for having celiac disease, the method comprising (a) determining a level of IP-10 in a sample comprising a T cell from the subject, which sample has been contacted with a composition comprising at least one gluten peptide; and (b) assessing whether or not the subject has or is at risk of having Celiac disease.

In some embodiments, the determining step comprises (i) contacting the sample comprising the T cell with the composition comprising at least one gluten peptide; and (ii) measuring the level of IP-10 in the sample comprising the T cell after the contacting. In some embodiments, measuring the level of IP-10 comprises an enzyme-linked immunosorbent assay (ELISA) or a multiplex bead-based immunoassay.

In some embodiments of any one of the methods provided, the method further comprises: (c) comparing the level of IP-10 in the sample with a control level of IP-10. In some embodiments, the assessing comprises (i) identifying the subject as having or at risk of having Celiac disease if the IP-10 level is elevated compared to a control level of IP-10; or (ii) not having or not at risk of having Celiac disease if the IP-10 level is reduced compared to the control level of IP-10 or the same as the control level of IP-10.

In some embodiments, the control level of IP-10 is a pre-determined threshold. In some embodiments, the control level of IP-10 is the level of IP-10 in another sample comprising a T cell obtained from the subject that is not contacted with the composition comprising at least one gluten peptide. In some embodiments, the sample comprising the T cell is a sample that comprises whole blood or peripheral blood mononuclear cells.

In some embodiments, the composition comprises at least one (e.g., one, two, three, four, five, six, seven, eight, nine, ten, eleven, twelve, thirteen, fourteen, fifteen, or sixteen) peptide(s), the at least one (e.g., one, two, three, four, five, six, seven, eight, nine, ten, eleven, twelve, thirteen, fourteen, fifteen, or sixteen) peptide(s) comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23). In some embodiments, the composition comprises at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23).

In some embodiments, the composition comprises (or consists of) at least one (or consists of) of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 15);

(k) a eleventh peptide comprising the amino acid sequence QGYYPTSPQ (SEQ ID NO: 16);

(l) a twelfth peptide comprising the amino acid sequence EQPEQPFPE (SEQ ID NO: 17);

(m) a thirteenth peptide comprising the amino acid sequence PFSEQEQPV (SEQ ID NO: 18);

(n) a fourteenth peptide comprising the amino acid sequence PYPQPELPY (SEQ ID NO: 19);

(o) a fifteenth peptide comprising the amino acid sequence EQPFPEQPI (SEQ ID NO: 20) and the amino acid sequence PFPEQPIPE (SEQ ID NO: 21); and

(p) a sixteenth peptide comprising the amino acid sequence PYPEQPQPF (SEQ ID NO: 22) and the amino acid sequence PQPYPEQPQ (SEQ ID NO: 23).

In some embodiments:

(a) the first peptide comprises the amino acid sequence PFPQPELPYPQP (SEQ ID NO: 24);

(b) the second peptide comprises the amino acid sequence PFPQPEQPFPWQ (SEQ ID NO: 25);

(c) the third peptide comprises the amino acid sequence EQPIPEQPQPYP (SEQ ID NO: 26);

(d) the fourth peptide comprises the amino acid sequence PFPQPEQPIPVQ (SEQ ID NO: 27);

(e) the fifth peptide comprises the amino acid sequence PEQPIPVQPEQS (SEQ ID NO: 28);

(f) the sixth peptide comprises the amino acid sequence PFPQPEQPTPIQ (SEQ ID NO: 29);

(g) the seventh peptide comprises the amino acid sequence PEQPTPIQPEQP (SEQ ID NO: 30);

(h) the eighth peptide comprises the amino acid sequence PFPQPEQPFPLQ (SEQ ID NO: 31);

(i) the ninth peptide comprises the amino acid sequence PEQPFPLQPEQP (SEQ ID NO: 32);

(j) the tenth peptide comprises the amino acid sequence GEGSFQPSQENP (SEQ ID NO: 33);

(k) the eleventh peptide comprises the amino acid sequence QQGYYPTSPQQS (SEQ ID NO: 34);

(l) the twelfth peptide comprises the amino acid sequence PEQPEQPFPEQP (SEQ ID NO: 35);

(m) the thirteenth peptide comprises the amino acid sequence PPFSEQEQPVLP (SEQ ID NO: 36);

(n) the fourteenth peptide comprises the amino acid sequence PYPQPELPYPQP (SEQ ID NO: 37);

(o) the fifteenth peptide comprises the amino acid sequence EQPFPEQPIPEQ (SEQ ID NO: 38); and

(p) the sixteenth peptide comprises the amino acid sequence PQPYPEQPQPFP (SEQ ID NO: 39).

In some embodiments, the composition comprises (or consists of) at least four (e.g., four, five, six, seven, eight, nine, ten, eleven, twelve, thirteen, fourteen, fifteen or sixteen) of the peptides. In some embodiments, the composition comprises (or consists of) the peptides in (a)-(p).

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 12 to 30 amino acids in length. In some embodiments, each of the peptides is 13 amino acids in length.

In some embodiments of any one of the compositions provided, each of the peptides are present in an amount of 2.5 ug/mL in the composition. In some embodiments of any one of the compositions provided, each of the peptides are present in an amount of 5 ug/mL in the composition. In some embodiments of any one of the compositions provided, each of the peptides are present in an amount of 10 ug/mL in the composition. In some embodiments of any one of the compositions provided, each of the peptides are present in an amount of 20 ug/mL in the composition. In some embodiments of any one of the compositions provided, each of the peptides are present in an amount of 25 ug/mL in the composition. In some embodiments of any one of the compositions provided, each of the peptides are present in an amount of 50 ug/mL in the composition. In some embodiments of any one of the compositions provided, each of the peptides are present in an amount of 5 uM (micromolar) in the composition. In some embodiments of any one of the compositions provided, each of the peptides are present in an amount of 10 uM in the composition. In some embodiments of any one of the compositions provided, each of the peptides are present in an amount of 25 uM in the composition. In some embodiments of any one of the compositions provided, each of the peptides are present in an amount of 50 uM in the composition.

In some embodiments, the composition comprises at least one (e.g., one, two, three, four, five, six, seven, eight, nine, ten, eleven, twelve, thirteen, fourteen, fifteen, or sixteen) peptide(s), the at least one (e.g., one, two, three, four, five, six, seven, eight, nine, ten, eleven, twelve, thirteen, fourteen, fifteen, or sixteen) peptide(s) comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23). In some embodiments, the composition comprises at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23). In some embodiments, the compositions comprises at least one (e.g., one, two, three, four, five, six, seven, eight, nine, ten, eleven, twelve, thirteen, fourteen, fifteen, or sixteen) peptide(s) comprising the amino acid sequences PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6) and at least one (e.g., one, two, three, four, five, six, seven, eight, nine, ten, eleven, twelve, thirteen, fourteen, fifteen, sixteen, seventeen, eighteen, nineteen, twenty, twenty-one or more) further amino acid sequence selected from PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23). In some embodiments, the composition comprises at least one peptide comprising the amino acid sequences EQPFPEQPI (SEQ ID NO: 20), PFPEQPIPE (SEQ ID NO: 21), EQPIPEQPQ (SEQ ID NO: 5), and PIPEQPQPY (SEQ ID NO: 6) (e.g., the composition comprises at least one peptide comprising the amino acid sequence PEQPFPEQPIPEQPQPYP (SEQ ID NO: 44)).

In some embodiments, the composition comprises (or consists of) at least one of (e.g., one, two, three, four, five, six, seven, eight, nine, ten, eleven, twelve, thirteen, fourteen, fifteen, or sixteen):

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 40);

(h) an eighth peptide comprising the amino acid sequence PYPEQPQPF (SEQ ID NO: 22);

(i) a ninth peptide comprising the amino acid sequence PFPEQPEQI (SEQ ID NO: 42);

(j) a tenth peptide comprising the amino acid sequence EGSFQPSQE (SEQ ID NO: 15);

(k) an eleventh peptide comprising the amino acid sequence QGYYPTSPQ (SEQ ID NO: 16);

(l) a twelfth peptide comprising the amino acid sequence EQPEQPFPE (SEQ ID NO: 17) and the amino acid sequence EQPFPEQPQ (SEQ ID NO: 41);

(m) a thirteenth peptide comprising the amino acid sequence PFSEQEQPV (SEQ ID NO: 18);

(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 19);

(p) a sixteenth peptide comprising the amino acid sequence PFPQPELPY (SEQ ID NO: 1) and the amino acid sequence PQPELPYPY (SEQ ID NO: 43);

(q) a seventeenth peptide comprising the amino acid sequence EQPFPEQPI (SEQ ID NO: 20) and the amino acid sequence PFPEQPIPE (SEQ ID NO: 21); and

(r) an eighteenth peptide comprising the amino acid sequence PQPYPEQPQ (SEQ ID NO: 23) and the amino acid sequence PYPEQPQPF (SEQ ID NO: 22).

In some embodiments,

(a) the first peptide comprises the amino acid sequence LQPFPQPELPYPQPQ (SEQ ID NO: 45);

(b) the second peptide comprises the amino acid sequence QPFPQPEQPFPWQP (SEQ ID NO: 46);

(c) the third peptide comprises the amino acid sequence PEQPIPEQPQPYPQQ (SEQ ID NO: 47);

(d) the fourth peptide comprises the amino acid sequence QPFPQPEQPIPVQPEQS (SEQ ID NO: 48);

(e) the fifth peptide comprises the amino acid sequence QPFPQPEQPTPIQPEQP (SEQ ID NO: 49);

(f) the sixth peptide comprises the amino acid sequence QPFPQPEQPFPLQPEQP (SEQ ID NO: 50);

(g) the seventh peptide comprises the amino acid sequence QPFPQPEQPFSQQ (SEQ ID NO: 51);

(h) the eighth peptide comprises the amino acid sequence PQPYPEQPQPFPQQ (SEQ ID NO: 52);

(i) the ninth peptide comprises the amino acid sequence QPFPEQPEQIIPQQP (SEQ ID NO: 53);

(j) the tenth peptide comprises the amino acid sequence SGEGSFQPSQENPQ (SEQ ID NO: 54);

(k) the eleventh peptide comprises the amino acid sequence GQQGYYPTSPQQSG (SEQ ID NO: 55);

(l) the twelfth peptide comprises the amino acid sequence PEQPEQPFPEQPQQ (SEQ ID NO: 56);

(m) the thirteenth peptide comprises the amino acid sequence QPPFSEQEQPVLPQ (SEQ ID NO: 57);

(n) the fourteenth peptide comprises the amino acid sequence PEQPFPEQPIPEQPQPYP (SEQ ID NO: 44);

(o) the fifteenth peptide comprises the amino acid sequence QPYPQPELPYPQPQ (SEQ ID NO: 58);

(p) the sixteenth peptide comprises the amino acid sequence QPFPQPELPYPYPQ (SEQ ID NO: 59);

(q) the seventeenth peptide comprises the amino acid sequence PQEQPFPEQPIPEQP (SEQ ID NO: 60); and

(r) the eighteenth peptide comprises the amino acid sequence QPQPYPEQPQPFPQQ (SEQ ID NO: 61).

In some embodiments, the composition comprises (or consists of) the first, second, and third peptide. In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, tenth, eleventh, twelfth, thirteenth, fifteenth, seventeenth, and eighteenth peptides.

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 14 to 20 amino acids in length.

In some embodiments of any one of the compositions provided, a composition comprises (or consists of) any one of the peptide pools as described in the examples provided. In some embodiments, a composition comprising the epitopes of any one of the peptide pools of the examples is provided. In some embodiments of any one of the compositions, the peptides or epitopes are in equimolar amounts.

In some embodiments of any one of the compositions provided, each of the peptides are present in an amount of 2.5 ug/mL in the composition. In some embodiments of any one of the compositions provided, each of the peptides are present in an amount of 5 ug/mL in the composition. In some embodiments of any one of the compositions provided, each of the peptides are present in an amount of 10 ug/mL in the composition. In some embodiments of any one of the compositions provided, each of the peptides are present in an amount of 20 ug/mL in the composition. In some embodiments of any one of the compositions provided, each of the peptides are present in an amount of 25 ug/mL in the composition. In some embodiments of any one of the compositions provided, each of the peptides are present in an amount of 50 ug/mL in the composition. In some embodiments of any one of the compositions provided, each of the peptides are present in an amount of 5 uM (micromolar) in the composition. In some embodiments of any one of the compositions provided, each of the peptides are present in an amount of 10 uM in the composition. In some embodiments of any one of the compositions provided, each of the peptides are present in an amount of 25 uM in the composition. In some embodiments of any one of the compositions provided, each of the peptides are present in an amount of 50 uM in the composition.

In some embodiments, the composition comprises (or consists of) at least one (e.g., one, two, three, four, five, six, seven, eight, nine, ten, eleven, twelve, thirteen, fourteen, fifteen, or sixteen) of:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 63) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 40);

(viii) an eighth peptide comprising the amino acid sequence PYPEQPQPF (SEQ ID NO: 22);

(ix) a ninth peptide comprising the amino acid sequence PFPEQPEQIIP (SEQ ID NO: 64);

(x) a tenth peptide comprising the amino acid sequence EGSFQPSQE (SEQ ID NO: 15);

(xi) an eleventh peptide comprising the amino acid sequence QGYYPTSPQ (SEQ ID NO: 16);

(xii) a twelfth peptide comprising the amino acid sequence EQPEQPFPEQPQ (SEQ ID NO: 65);

(xiii) a thirteenth peptide comprising the amino acid sequence PFSEQEQPV (SEQ ID NO: 18);

(xiv) a fourteenth peptide comprising the amino acid sequence EQPFPEQPI (SEQ ID NO: 20) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 19);

(xvi) a sixteenth peptide comprising the amino acid sequence PFPQPELPY (SEQ ID NO: 1) and the amino acid sequence PQPELPYPY (SEQ ID NO: 43); and

(xvii) a seventeenth peptide comprising the amino acid sequence EQPFPEQPI (SEQ ID NO: 20).

In some embodiments, (i) the first peptide comprises the amino acid sequence LQPFPQPELPYPQPQ (SEQ ID NO: 45); (ii) the second peptide comprises the amino acid sequence QPFPQPEQPFPWQP (SEQ ID NO: 46); (iii) the third peptide comprises the amino acid sequence PEQPIPEQPQPYPQQ (SEQ ID NO: 47); (iv) the fourth peptide comprises the amino acid sequence QPFPQPEQPIPVQPEQS (SEQ ID NO: 48); (v) the fifth peptide comprises the amino acid sequence QPFPQPEQPTPIQPEQP (SEQ ID NO: 49); (vi) the sixth peptide comprises the amino acid sequence QPFPQPEQPFPLQPEQP (SEQ ID NO: 50); (vii) the seventh peptide comprises the amino acid sequence QPFPQPEQPFSQQ (SEQ ID NO: 51); (viii) the eighth peptide comprises the amino acid sequence PQPYPEQPQPFPQQ (SEQ ID NO: 52); (ix) the ninth peptide comprises the amino acid sequence QPFPEQPEQIIPQQP (SEQ ID NO: 53); (x) the tenth peptide comprises the amino acid sequence SGEGSFQPSQENPQ (SEQ ID NO: 54); (xi) the eleventh peptide comprises the amino acid sequence GQQGYYPTSPQQSG (SEQ ID NO: 55); (xii) the twelfth peptide comprises the amino acid sequence PEQPEQPFPEQPQQ (SEQ ID NO: 56); (xiii) the thirteenth peptide comprises the amino acid sequence QPPFSEQEQPVLPQ (SEQ ID NO: 57); (xiv) the fourteenth peptide comprises the amino acid sequence PEQPFPEQPIPEQPQPYP (SEQ ID NO: 44); (xv) the fifteenth peptide comprises the amino acid sequence QPYPQPELPYPQPQ (SEQ ID NO: 58); (xvi) the sixteenth peptide comprises the amino acid sequence QPFPQPELPYPYPQ (SEQ ID NO: 59); and (xvii) the seventeenth peptide comprises the amino acid sequence EQPFPEQPI (SEQ ID NO: 20).

In some embodiments, the composition comprises (or consists of) the first, second, and third peptide. In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides.

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 14 to 20 amino acids in length.

In some embodiments of any one of the compositions provided, each of the peptides are present in an amount of 2.5 ug/mL in the composition. In some embodiments of any one of the compositions provided, each of the peptides are present in an amount of 5 ug/mL in the composition. In some embodiments of any one of the compositions provided, each of the peptides are present in an amount of 10 ug/mL in the composition. In some embodiments of any one of the compositions provided, each of the peptides are present in an amount of 20 ug/mL in the composition. In some embodiments of any one of the compositions provided, each of the peptides are present in an amount of 25 ug/mL in the composition. In some embodiments of any one of the compositions provided, each of the peptides are present in an amount of 50 ug/mL in the composition. In some embodiments of any one of the compositions provided, each of the peptides are present in an amount of 5 uM (micromolar) in the composition. In some embodiments of any one of the compositions provided, each of the peptides are present in an amount of 10 uM in the composition. In some embodiments of any one of the compositions provided, each of the peptides are present in an amount of 25 uM in the composition. In some embodiments of any one of the compositions provided, each of the peptides are present in an amount of 50 uM in the composition.

In some embodiments of any one of the methods provided, the method further comprises treating the subject if identified as having or at risk of having Celiac disease or providing information to the subject about a treatment. In some embodiments of any one of the methods provided, the method further comprises a step of recommending a gluten-free diet if the subject is identified as having or at risk of having Celiac disease or providing information to the subject about such a diet. In some embodiments of any one of the methods provided, the method further comprises performing other testing. In some embodiments, the other testing comprises performing a serology assay, genotyping, and/or an intestinal biopsy.

In some embodiments, the subject is HLA-DQ2.5 positive, HLA-DQ2.2 positive and/or HLA-DQ8 positive. In some embodiments, the subject is HLA-DQ2.5 positive.

In some embodiments of any one of the methods provided, the method further comprises administering a composition comprising wheat, rye, and/or barley, or a composition comprising a gluten peptide, to the subject at least once a day for one day. In some embodiments of any one of the methods provided, the method further comprises administering a composition comprising wheat, rye, and/or barley to the subject at least once a day for two days.

In some embodiments of any one of the methods provided, the method further comprises administering a composition to the subject comprising 10 micrograms of the first peptide and an equimolar amount of each of the second and third peptides; 15 micrograms of the first peptide and an equimolar amount of each of the second and third peptides; 20 micrograms of the first peptide and an equimolar amount of each of the second and third peptides; or 50 micrograms of the first peptide and an equimolar amount of each of the second and third peptides. In some embodiments, the composition is administered to the subject once. In some embodiments, the composition is administered intradermally.

In some embodiments of any one of the methods provided, the subject has not undergone a gluten challenge within 1 week of the sample being obtained from the subject. In some embodiments of any one of the methods provided, the subject has a level of IFN-gamma that is reduced or the same as a control level of IFN-gamma. In some embodiments of any one of the methods provided, the level of IFN-gamma is not statically significantly higher than the control level of IFN-gamma. In some embodiments of any one of the methods provided, the control level of IFN-gamma is 7.2 pg/mL. In some embodiments of any one of the methods provided, the subject is on a diet that contains gluten.

In some embodiments of any one of the methods provided herein, the method further comprises recording the level(s), the result(s) of the assessing and/or the treatment, or suggestion for treatment, based on the assessing.

Other aspects of the disclosure relate to a kit comprising a binding partner for IP-10 and any one of the compositions provided, such as a composition comprising (or consisting of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 40);

(h) an eighth peptide comprising the amino acid sequence PYPEQPQPF (SEQ ID NO: 22);

(i) a ninth peptide comprising the amino acid sequence PFPEQPEQI (SEQ ID NO: 42);

(j) a tenth peptide comprising the amino acid sequence EGSFQPSQE (SEQ ID NO: 15);

(k) an eleventh peptide comprising the amino acid sequence QGYYPTSPQ (SEQ ID NO: 16);

(l) a twelfth peptide comprising the amino acid sequence EQPEQPFPE (SEQ ID NO: 17) and the amino acid sequence EQPFPEQPQ (SEQ ID NO: 41);

(m) a thirteenth peptide comprising the amino acid sequence PFSEQEQPV (SEQ ID NO: 18);

(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 19);

(p) a sixteenth peptide comprising the amino acid sequence PFPQPELPY (SEQ ID NO: 1) and the amino acid sequence PQPELPYPY (SEQ ID NO: 43);

(q) a seventeenth peptide comprising the amino acid sequence EQPFPEQPI (SEQ ID NO: 20) and the amino acid sequence PFPEQPIPE (SEQ ID NO: 21); and

(r) an eighteenth peptide comprising the amino acid sequence PQPYPEQPQ (SEQ ID NO: 23) and the amino acid sequence PYPEQPQPF (SEQ ID NO: 22).

In some embodiments,

(a) the first peptide comprises the amino acid sequence LQPFPQPELPYPQPQ (SEQ ID NO: 45);

(b) the second peptide comprises the amino acid sequence QPFPQPEQPFPWQP (SEQ ID NO: 46);

(c) the third peptide comprises the amino acid sequence PEQPIPEQPQPYPQQ (SEQ ID NO: 47);

(d) the fourth peptide comprises the amino acid sequence QPFPQPEQPIPVQPEQS (SEQ ID NO: 48);

(e) the fifth peptide comprises the amino acid sequence QPFPQPEQPTPIQPEQP (SEQ ID NO: 49);

(f) the sixth peptide comprises the amino acid sequence QPFPQPEQPFPLQPEQP (SEQ ID NO: 50);

(g) the seventh peptide comprises the amino acid sequence QPFPQPEQPFSQQ (SEQ ID NO: 51);

(h) the eighth peptide comprises the amino acid sequence PQPYPEQPQPFPQQ (SEQ ID NO: 52);

(i) the ninth peptide comprises the amino acid sequence QPFPEQPEQIIPQQP (SEQ ID NO: 53);

(j) the tenth peptide comprises the amino acid sequence SGEGSFQPSQENPQ (SEQ ID NO: 54);

(k) the eleventh peptide comprises the amino acid sequence GQQGYYPTSPQQSG (SEQ ID NO: 55);

(l) the twelfth peptide comprises the amino acid sequence PEQPEQPFPEQPQQ (SEQ ID NO: 56);

(m) the thirteenth peptide comprises the amino acid sequence QPPFSEQEQPVLPQ (SEQ ID NO: 57);

(n) the fourteenth peptide comprises the amino acid sequence PEQPFPEQPIPEQPQPYP (SEQ ID NO: 44);

(o) the fifteenth peptide comprises the amino acid sequence QPYPQPELPYPQPQ (SEQ ID NO: 58);

(p) the sixteenth peptide comprises the amino acid sequence QPFPQPELPYPYPQ (SEQ ID NO: 59);

(q) the seventeenth peptide comprises the amino acid sequence PQEQPFPEQPIPEQP (SEQ ID NO: 60); and

(r) the eighteenth peptide comprises the amino acid sequence QPQPYPEQPQPFPQQ (SEQ ID NO: 61).

In some embodiments, the composition comprises (or consists of) the first, second, and third peptides.

In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 20 ug/mL in the composition. In some embodiments of any one of the kits provided, each of the peptides are present in an amount of 50 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments, the composition comprises (or consists of) the first, second, third, fourth, fifth, sixth, seventh, eighth, ninth, tenth, eleventh, twelfth, and thirteenth peptides or the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, tenth, eleventh, twelfth, thirteenth, fifteenth, seventeenth, and eighteenth peptides. In some embodiments of any one of the kits provided, each of the peptides are present in an amount of 2.5 ug/mL in the composition. In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 25 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments of any one of the kits provided, each of the peptides comprises an N-terminal pyroglutamate and/or a C-terminal amide group.

In some embodiments of any one of the kits provided, the kit further comprises a composition comprising wheat, rye, and/or barley, such a foodstuff. In some embodiments of any one of the kits provided, the kit further comprises a second binding partner for IP-10, such as a secondary antibody.

Other aspects of the disclosure relate to a kit comprising a binding partner for IP-10 and a composition comprising (or consists of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence PIPEQPQPY (SEQ ID NO: 6);

(d) a fourth peptide comprising the amino acid sequence PFPQPEQPIP (SEQ ID NO: 62) and the amino acid sequence EQPIPVQPE (SEQ ID NO: 9);

(e) a fifth peptide comprising the amino acid sequence PFPQPEQPTPI (SEQ ID NO: 63) and the amino acid sequence EQPTPIQPE (SEQ ID NO: 12);

(f) a sixth peptide comprising the amino acid sequence PQPEQPFPL (SEQ ID NO: 13) and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 40);

(h) an eighth peptide comprising the amino acid sequence PYPEQPQPF (SEQ ID NO: 22);

(i) a ninth peptide comprising the amino acid sequence PFPEQPEQIIP (SEQ ID NO: 64);

(j) a tenth peptide comprising the amino acid sequence EGSFQPSQE (SEQ ID NO: 15);

(k) an eleventh peptide comprising the amino acid sequence QGYYPTSPQ (SEQ ID NO: 16);

(l) a twelfth peptide comprising the amino acid sequence EQPEQPFPEQPQ (SEQ ID NO: 65);

(m) a thirteenth peptide comprising the amino acid sequence PFSEQEQPV (SEQ ID NO: 18);

(n) a fourteenth peptide comprising the amino acid sequence EQPFPEQPI (SEQ ID NO: 20) and PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 19);

(p) a sixteenth peptide comprising the amino acid sequence PFPQPELPY (SEQ ID NO: 1) and the amino acid sequence PQPELPYPY (SEQ ID NO: 43); and

(q) a seventeenth peptide comprising the amino acid sequence EQPFPEQPI (SEQ ID NO: 20).

In some embodiments, (a) the first peptide comprises the amino acid sequence LQPFPQPELPYPQPQ (SEQ ID NO: 45); (b) the second peptide comprises the amino acid sequence QPFPQPEQPFPWQP (SEQ ID NO: 46); (c) the third peptide comprises the amino acid sequence PEQPIPEQPQPYPQQ (SEQ ID NO: 47); (d) the fourth peptide comprises the amino acid sequence QPFPQPEQPIPVQPEQS (SEQ ID NO: 48); (e) the fifth peptide comprises the amino acid sequence QPFPQPEQPTPIQPEQP (SEQ ID NO: 49); (f) the sixth peptide comprises the amino acid sequence QPFPQPEQPFPLQPEQP (SEQ ID NO: 50); (g) the seventh peptide comprises the amino acid sequence QPFPQPEQPFSQQ (SEQ ID NO: 51); (h) the eighth peptide comprises the amino acid sequence PQPYPEQPQPFPQQ (SEQ ID NO: 52); (i) the ninth peptide comprises the amino acid sequence QPFPEQPEQIIPQQP (SEQ ID NO: 53); (j) the tenth peptide comprises the amino acid sequence SGEGSFQPSQENPQ (SEQ ID NO: 54); (k) the eleventh peptide comprises the amino acid sequence GQQGYYPTSPQQSG (SEQ ID NO: 55); (1) the twelfth peptide comprises the amino acid sequence PEQPEQPFPEQPQQ (SEQ ID NO: 56); (m) the thirteenth peptide comprises the amino acid sequence QPPFSEQEQPVLPQ (SEQ ID NO: 57); (n) the fourteenth peptide comprises the amino acid sequence PEQPFPEQPIPEQPQPYP (SEQ ID NO: 44); (o) the fifteenth peptide comprises the amino acid sequence QPYPQPELPYPQPQ (SEQ ID NO: 58); (p) the sixteenth peptide comprises the amino acid sequence QPFPQPELPYPYPQ (SEQ ID NO: 59); and (q) the seventeenth peptide comprises the amino acid sequence EQPFPEQPI (SEQ ID NO: 20).

In some embodiments, the composition comprises (or consists of) the first, second, and third peptides.

In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 20 ug/mL in the composition. In some embodiments of any one of the kits provided, each of the peptides are present in an amount of 50 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments, the composition comprises (or consists of) the first, second, third, fourth, fifth, sixth, seventh, eighth, ninth, tenth, eleventh, twelfth, and thirteenth peptides or the composition comprises (or consists of) the second, fourth, fifth, sixth, seventh, eighth, ninth, tenth, eleventh, twelfth, thirteenth, fourteenth, fifteenth, and sixteenth peptides. In some embodiments of any one of the kits provided, each of the peptides are present in an amount of 2.5 ug/mL in the composition. In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 25 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments of any one of the kits provided, each of the peptides comprises an N-terminal pyroglutamate and/or a C-terminal amide group.

In some embodiments of any one of the kits provided, the composition comprises at least one (e.g., one, two, three, four, five, six, seven, eight, nine, ten, eleven, twelve, thirteen, fourteen, fifteen, or sixteen) peptide(s), the at least one (e.g., one, two, three, four, five, six, seven, eight, nine, ten, eleven, twelve, thirteen, fourteen, fifteen, or sixteen) peptide(s) comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23). In some embodiments, the composition comprises (or consists of) at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23).

In some embodiments of any one of the kits provided, the composition comprises (or consists of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9); (f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 15);

(k) a eleventh peptide comprising the amino acid sequence QGYYPTSPQ (SEQ ID NO: 16);

(l) a twelfth peptide comprising the amino acid sequence EQPEQPFPE (SEQ ID NO: 17);

(m) a thirteenth peptide comprising the amino acid sequence PFSEQEQPV (SEQ ID NO: 18);

(n) a fourteenth peptide comprising the amino acid sequence PYPQPELPY (SEQ ID NO: 19);

(o) a fifteenth peptide comprising the amino acid sequence EQPFPEQPI (SEQ ID NO: 20) and the amino acid sequence PFPEQPIPE (SEQ ID NO: 21); and

(p) a sixteenth peptide comprising the amino acid sequence PYPEQPQPF (SEQ ID NO: 22) and the amino acid sequence PQPYPEQPQ (SEQ ID NO: 23).

In some embodiments of any one of the kits provided,

(a) the first peptide comprises the amino acid sequence PFPQPELPYPQP (SEQ ID NO: 24);

(b) the second peptide comprises the amino acid sequence PFPQPEQPFPWQ (SEQ ID NO: 25);

(c) the third peptide comprises the amino acid sequence EQPIPEQPQPYP (SEQ ID NO: 26);

(d) the fourth peptide comprises the amino acid sequence PFPQPEQPIPVQ (SEQ ID NO: 27);

(e) the fifth peptide comprises the amino acid sequence PEQPIPVQPEQS (SEQ ID NO: 28);

(f) the sixth peptide comprises the amino acid sequence PFPQPEQPTPIQ (SEQ ID NO: 29);

(g) the seventh peptide comprises the amino acid sequence PEQPTPIQPEQP (SEQ ID NO: 30);

(h) the eighth peptide comprises the amino acid sequence PFPQPEQPFPLQ (SEQ ID NO: 31);

(i) the ninth peptide comprises the amino acid sequence PEQPFPLQPEQP (SEQ ID NO: 32);

(j) the tenth peptide comprises the amino acid sequence GEGSFQPSQENP (SEQ ID NO: 33);

(k) the eleventh peptide comprises the amino acid sequence QQGYYPTSPQQS (SEQ ID NO: 34);

(l) the twelfth peptide comprises the amino acid sequence PEQPEQPFPEQP (SEQ ID NO: 35);

(m) the thirteenth peptide comprises the amino acid sequence PPFSEQEQPVLP (SEQ ID NO: 36);

(n) the fourteenth peptide comprises the amino acid sequence PYPQPELPYPQP (SEQ ID NO: 37);

(o) the fifteenth peptide comprises the amino acid sequence EQPFPEQPIPEQ (SEQ ID NO: 38); and

(p) the sixteenth peptide comprises the amino acid sequence PQPYPEQPQPFP (SEQ ID NO: 39).

In some embodiments of any one of the kits provided, the composition comprises (or consists of) at least four (e.g., four, five, six, seven, eight, nine, ten, eleven, twelve, thirteen, fourteen, fifteen or sixteen) of the peptides. In some embodiments of any one of the kits provided, the composition comprises (or consists of) the peptides in (a)-(p).

In some embodiments of any one of the kits provided, at least one of the peptides comprises an N-terminal pyroglutamate and/or a C-terminal amide group. In some embodiments of any one of the kits provided, each of the peptides comprises an N-terminal pyroglutamate and/or a C-terminal amide group. In some embodiments of any one of the kits provided, each of the peptides is less than full-length gluten. In some embodiments of any one of the kits provided, each of the peptides is independently between 8 to 50 amino acids in length. In some embodiments, each of the peptides is independently between 10 to 30 amino acids in length. In some embodiments, each of the peptides is independently between 12 to 30 amino acids in length. In some embodiments, each of the peptides is 13 amino acids in length.

In some embodiments of any one of the kits provided, each of the peptides are present in an amount of 2.5 ug/mL in the composition. In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 20 ug/mL in the composition. In some embodiments of any one of the kits provided, each of the peptides are present in an amount of 25 ug/mL in the composition. In some embodiments of any one of the kits provided, each of the peptides are present in an amount of 50 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments of any one of the kits provided, the kit further comprises a second composition comprising the first, second and third peptides, wherein the second composition contains 10 micrograms of the first peptide and an equimolar amount of each of the second and third peptides; 15 micrograms of the first peptide and an equimolar amount of each of the second and third peptides; 20 micrograms of the first peptide and an equimolar amount of each of the second and third peptides; or 50 micrograms of the first peptide and an equimolar amount of each of the second and third peptides. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments of any one of the kits provided, the kit further comprises a composition comprising wheat, rye, and/or barley, such a foodstuff. In some embodiments of any one of the kits provided, the kit further comprises a second binding partner for IP-10, such as a secondary antibody.

In some embodiments of any one of the compositions, methods or kits provided, the peptides in a composition each consist of the recited amino acid sequence(s).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-D are a series of four graphs showing the amount of interferon-gamma (IFNγ) and IP-10 in whole blood samples collected from multiple subjects one day before beginning a three-day oral gluten challenge (day 0) and six days after beginning the oral gluten challenge (day 6). The whole blood samples were contacted with medium or peptide pool 1 and the level of IFNγ or IP-10 was measured by MAGPIX® assay (Luminex). The subjects are indicated as follows: subject 1: filled-in circle, subject 2: filled-in square, subject 3: filled-in point-up triangle, subject 4: filled-in point-down triangle, subject 5: filled-in diamond, subject 6: unfilled circle, subject 7: unfilled square, subject 8: unfilled point-up triangle, subject 9: unfilled point-down triangle, and subject 10: unfilled diamond.

FIG. 2A is a graph showing the amount of IFNγ as measured by ELISA in blood samples. The amount is shown as the amount in the blood sample contacted with peptide pool 1 minus the amount in the blood sample contacted with medium (pool 1 medium). Individual points indicate blood drawn from each of 10 subjects.

FIG. 2B is a graph showing the amount of IFNγ as measured by MAGPIX® in blood samples. The amount is shown as the amount in the blood sample contacted with peptide pool 1 minus the amount in the blood sample contacted with medium (pool 1 medium). Individual points indicate blood drawn from each of 10 subjects.

FIG. 2C is a graph showing the amount of IP-10 as measured by MAGPIX® in blood samples. The amount is shown as the amount in a blood sample contacted with peptide pool 1 minus the amount in a blood sample contacted with medium (pool 1 medium). Individual points indicate blood drawn from each of 10 subjects.

FIG. 3A is a graph showing the correlation between IFNγ (IFNg) ELISA v. IFNγ MAGPIX® at six days after beginning the oral gluten challenge (day 6).

FIG. 3B is a graph showing the correlation between IFNγ (IFNg) MAGPIX® v. IP-10 MAGPIX® at six days after beginning the oral gluten challenge (day 6).

FIGS. 4A-C show the levels of IP-10, IFNγ, and the number of IFNγ SFUs (spot forming units) in blood from exemplary subject 1. The blood was contacted with medium (negative control), CEF (human CMV, EBV and influenza virus, positive control), peptide pool 1, peptide pool 2, peptide pool 3, or total gluten peptide pool. For each graph in FIGS. 4A-C and also in each graph in FIGS. 5A-13C, the X-axis labels from left to right are: Day 0 medium, Day 0 CEF, Day 6 medium, Day 6 CEF, Day 0 medium, Day 0 Pool 1 3×10 ug/ml, Day 6 medium, Day 6 pool 1 3×50 ug/mL, Day 6 pool 1 3×20 ug/mL, Day 6 pool 1 3×20 ug/mL, Day 6 pool 1 3×5 ug/mL, Day 0 medium, Day 0 Pool 2 13×5 ug/ml, Day 6 medium, Day 6 pool 2 13×25 ug/mL, Day 6 pool 2 13×10 ug/mL, Day 6 pool 2 13×5 ug/mL, Day 6 pool 2 13×2.5 ug/mL, Day 0 medium, Day 0 Pool 2 14×5 ug/ml, Day 6 medium, Day 6 pool 2 14×25 ug/mL, Day 6 pool 2 14×10 ug/mL, Day 6 pool 2 14×5 ug/mL, and Day 6 pool 2 14×2.5 ug/mL.

FIGS. 5A-C show the levels of IP-10, IFNγ, and the number of IFNγ SFUs (spot forming units) in blood from exemplary subject 2. The blood was contacted with medium (negative control), CEF (human CMV, EBV and influenza virus, positive control), peptide pool 1, peptide pool 2, peptide pool 3, or total gluten peptide pool.

FIGS. 6A-C show the levels of IP-10, IFNγ, and the number of IFNγ SFUs (spot forming units) in blood from exemplary subject 3. The blood was contacted with medium (negative control), CEF (human CMV, EBV and influenza virus, positive control), peptide pool 1, peptide pool 2, peptide pool 3, or total gluten peptide pool.

FIGS. 7A-C show the levels of IP-10, IFNγ, and the number of IFNγ SFUs (spot forming units) in blood from exemplary subject 4. The blood was contacted with medium (negative control), CEF (human CMV, EBV and influenza virus, positive control), peptide pool 1, peptide pool 2, peptide pool 3, or total gluten peptide pool.

FIGS. 8A-C show the levels of IP-10, IFNγ, and the number of IFNγ SFUs (spot forming units) in blood from exemplary subject 5. The blood was contacted with medium (negative control), CEF (human CMV, EBV and influenza virus, positive control), peptide pool 1, peptide pool 2, peptide pool 3, or total gluten peptide pool.

FIGS. 9A-C show the levels of IP-10, IFNγ, and the number of IFNγ SFUs (spot forming units) in blood from exemplary subject 6. The blood was contacted with medium (negative control), CEF (human CMV, EBV and influenza virus, positive control), peptide pool 1, peptide pool 2, peptide pool 3, or total gluten peptide pool.

FIGS. 10A-C show the levels of IP-10, IFNγ, and the number of IFNγ SFUs (spot forming units) in blood from exemplary subject 7. The blood was contacted with medium (negative control), CEF (human CMV, EBV and influenza virus, positive control), peptide pool 1, peptide pool 2, peptide pool 3, or total gluten peptide pool.

FIGS. 11A-C show the levels of IP-10, IFNγ, and the number of IFNγ SFUs (spot forming units) in blood from exemplary subject 8. The blood was contacted with medium (negative control), CEF (human CMV, EBV and influenza virus, positive control), peptide pool 1, peptide pool 2, peptide pool 3, or total gluten peptide pool.

FIGS. 12A-C show the levels of IP-10, IFNγ, and the number of IFNγ SFUs (spot forming units) in blood from exemplary subject 9. The blood was contacted with medium (negative control), CEF (human CMV, EBV and influenza virus, positive control), peptide pool 1, peptide pool 2, peptide pool 3, or total gluten peptide pool.

FIGS. 13A-C show the levels of IP-10, IFNγ, and the number of IFNγ SFUs (spot forming units) in blood from exemplary subject 10. The blood was contacted with medium (negative control), CEF (human CMV, EBV and influenza virus, positive control), peptide pool 1, peptide pool 2, peptide pool 3, or total gluten peptide pool.

FIGS. 14A-D show IFNγ spot forming units (SFU) in an ELISpot of PBMCs in samples collected from subjects 6 days after commencing a 3 day oral gluten challenge.

FIGS. 15A-D show IFNγ spot forming units (SFU) in an ELISpot of PBMCs in samples collected from subjects 6 days after commencing a 3 day oral gluten challenge.

FIGS. 16A and B show IFNγ spot forming units (SFU) in an ELISpot of PBMCs in samples collected from subjects 6 days after commencing a 3 day oral gluten challenge.

FIG. 17 is a table showing IFNγ and IP-10 MAGPIX data in blood samples from subjects with Celiac disease after oral gluten challenge prior to a first treatment dose and after a last treatment dose with a peptide composition. The blood samples were contacted with a peptide composition or buffered solution (NIL) and the level IFNγ and IP-10 was measured by MAGPIX.

FIG. 18 is a table showing IFNγ and IP-10 MAGPIX data in blood samples from subjects with Celiac disease after oral gluten challenge prior to a first treatment dose and after a last treatment dose with a placebo. The blood samples were contacted with a peptide composition or buffered solution (NIL) and the level IFNγ and IP-10 was measured by MAGPIX.

FIGS. 19A-C are graphs that show responses to gluten peptide pools in cytokine release assays before (filled-in circles) and 6-days after (open circles) commencing oral gluten challenge in 10 HLA-DQ2.5+ subjects with celiac disease to medium only (Nil), P3 10 μg/mL, P14 5 μM, P13 5 μM, P71 10 μg/mL (P71), and CEF 1 μg/mL. Linked symbols represent individual subject data: spot forming units (SFU) per million PBMC in ELISpot assay, or the ratio of plasma cytokine concentration in whole blood incubated with antigen to medium only (stimulation index). Day-0 vs Day-6:*p<0.05 **p<0.01 by 2-tail Wilcoxon paired rank sum test.

FIGS. 20A-L are graphs that show Day-6 IFNγ ELISpot, whole blood (WB) IFNγ and IP-10 dose-responses to gluten peptide pools by subjects normalized against their response to P3 50 μg/mL. FIGS. 20A-D show ELISpot results normalized after subtraction of response to medium only for each of six subjects whose response to P3 50 μg/mL was at least 10 SFU per 1.2 million PBMC (3 wells) above medium only. Data shown are median +/− range from six subjects. FIGS. 20E-H show whole blood IFNγ release for seven subjects whose stimulation index to P3 50 μg/mL was SI>1.5. FIGS. 18I-L show whole blood IP-10 release for four subjects whose responses to P3 50 μg/mL were less than the maximal detectable limit, IP-10 levels in the other six subjects were all at or above the limit of quantitation. Statistical significance compared to P3 50 μg/mL for all 10 subjects assessed for IFNγ ELISpot and whole blood release are indicated by *p<0.05 or **p<0.01 (two-tail “Wilcoxon matched-pairs signed rank test”). Statistical significance of IP-10 responses were not formally tested due there being only four informative data sets.

FIGS. 21A-J are graphs that show several individual subject's measured plasma concentrations of IFNγ and IP-10 (pg/mL) before subtraction of response to medium alone in cytokine bead assay. Plasma was separated following 24 h whole blood incubation with individual gluten peptides (5 uM) or pools of peptides. Each graph is for blood collected six days after commencing oral gluten challenge for each of 10 subjects. r² values are for data points that were below the maximum level of quantitation for IP-10 is 10,000 pg/mL. FIGS. 21A-J are graphs for each of subjects 1-10, respectively.

FIGS. 22A-H are graphs that show the stimulation index and net concentration of IFNγ (FIGS. 22A-D) and IP-10 (FIGS. 20E-H) in plasma after subtraction of response to medium only in whole blood collected before (filled-in circles) and 6-days after (open circles) commencing oral gluten challenge in 10 HLADQ2.5+ subjects with celiac disease. Blood was incubated with one of four different peptide pools: (FIGS. 22A and E) P3 10 μg/mL, (FIGS. 22B and F) P14 5 μM, (FIGS. 22C and G) P13 5 μM, or (FIGS. 22D and H) P71 10 μg/mL.

FIGS. 23A-D are graphs that show IFNγ and IP-10 (pg/mL) in plasma from whole bloods samples incubated with medium alone. IFNγ and IP-10 measured in plasma from replicate blood samples collected on Day-6 in separate cytokine bead assay plates (inter-assay variation), or from blood collected before and after oral gluten challenge that was assessed in the same cytokine bead assay (temporal change). Ten subjects were studied on Day-0 and Day-6. Three sets of triplicate blood samples were incubated with medium and one set of triplicates was incubated in each of the duplicate plates on Day-6. One set of triplicate blood samples was incubated with medium on Day-0. Except for one plate, each blood sample incubated with medium yielded one plasma sample that was assessed in a single well in the cytokine bead assay. For the duplicate plates, corresponding wells were pooled. In one cytokine bead assay plate, IFNγ was measured in three triplicate plasmas from Day-6 and in one triplicate from Day-0. A further triplicate plasma sample from Day-6 was assessed in a second cytokine bead assay plate performed on the same day. Data points represent the mean of triplicates derived from three blood incubations.

FIG. 24 is a graph that shows IFNγ and IP-10 (pg/mL) in plasma from blood incubated with medium alone from 10 subjects. Plasma levels for both analytes were assessed in one set of triplicate blood incubations on Day-0 and from two sets of triplicate whole blood samples collected on Day-6. Each point represents the mean of triplicates.

FIG. 25A is a schedule of assessments for Cohorts A-E as outlined in Example 4.

FIG. 25B is a schedule of assessments for Cohorts F and G as outlined in Example 4.

FIG. 26 is a graph that shows the fold-change in IP-10 concentration in blood contacted with peptide pool 1, 3, or 4 compared to blood incubated with PBS alone.

### DETAILED DESCRIPTION

Celiac disease (CD, also sometimes referred to as cogiac disease, c(o)eliac sprue, non-tropical sprue, endemic sprue, gluten enteropathy or gluten-sensitive enteropathy, and gluten intolerance) is an autoimmune disorder of the small intestine caused by ingestion of gluten-containing foods that occurs in people of all ages, ranging from middle infancy onward, and affects approximately 1% of people in Europe and North America. Untreated Celiac disease is associated with increased risk of adenocarcinoma (small intestine cancer) and lymphoma of the small bowel (enteropathy-associated T-cell lymphoma), as well as other complications, such as ulcerative jejunitis (ulcer formation of the small bowel) and stricturing (narrowing as a result of scarring with obstruction of the bowel). In many of those affected, Celiac disease is unrecognized, but this clinical oversight is now being rectified with greater clinical awareness.

Celiac disease generally occurs in genetically susceptible individuals who possess either HLA-DQ2 encoded by HLA-DQAl *05 and HLA-DQBl *02 (accounting for about 90% of individuals), variants of HLA-DQ2, or HLA-DQ8. Without wishing to be bound by theory, such individuals are thought to mount an inappropriate HLA-DQ2- and/or DQ8-restricted CD4⁺ T cell-mediated immune response to peptides derived from the aqueous-insoluble proteins of wheat flour, gluten, and related proteins in rye and barley (herein referred to as gluten peptides).

Celiac disease is diagnosed by small bowel biopsy showing villous atrophy, crypt hyperplasia and raised intra-epithelial lymphocytes, and supported by the presence of celiac disease-specific serology (IgA specific for transglutaminase and/or IgA and IgG specific for deamidated gliadin peptide). Intestinal histology and serological abnormalities normalize or improve within weeks to months of adopting gluten-free diet. In general, celiac disease can be excluded if certain alleles encoding HLA-DQA1*05, DQB1*02 and DQB1*0302 are not present. In patients who have adopted a gluten-free diet (GFD) without definitive diagnosis, reintroduction of gluten into the diet has been necessary to make a firm diagnosis of celiac disease. Reintroduction of >3 g/day gluten (about 1.5 slices of wheat bread) daily leads to intestinal tissue damage in the majority of patients with celiac disease usually strictly adherent to gluten free diet.

Small bowel biopsy typically requires an endoscopy, which is inconvenient and may be inconclusive if biopsies are not performed at multiple sites in the duodenum, processed meticulously and interpreted correctly. Requiring small bowel biopsy may also delay treatment because of the importance of continuing to consume gluten until after the procedure. Furthermore, celiac disease cannot be diagnosed in patients who have excluded gluten from their diet if serology and histology do show typical diagnostic features.

Oral gluten challenge for 3 days mobilizes gluten-reactive T cells that can generally be measured six days after commencing the challenge. However, patients may not tolerate consuming gluten for three days and results are not available for a number of days. As described herein, it was surprisingly found that levels of IP-10 in blood samples contacted with pools of gluten peptides were significantly elevated, even when the blood samples were collected from subjects with celiac disease before undergoing an oral gluten challenge. IP-10 was also found to be highly elevated in blood samples collected after gluten challenge, after the blood samples were contacted with pools of gluten peptides. As a result, IP-10 appears to be a robust biomarker for celiac disease, which could be utilized without the need for a gluten challenge or with gluten challenge of less than 3 days.

Accordingly, aspects of the disclosure relate to methods of identifying a subject having or at risk for having celiac disease by determining a level of IP-10 in a sample from the subject.

### Methods

One aspect of the disclosure relates to methods useful for diagnosis of a subject, such as a subject having or suspected of having Celiac disease. The methods involve determining (e.g., measuring) a level of IP-10 in a sample from the subject.

In some embodiments, the method comprises determining a level of IP-10 in a sample comprising a T cell from a subject having or suspected of having Celiac disease, wherein the sample has been contacted (e.g., contacted ex vivo) with any one of the compositions comprising at least one gluten peptide as described herein prior to the determining; and assessing whether or not the subject has or is at risk of having Celiac disease. The composition comprising at least one gluten peptide may be any one of the compositions provided in the Examples. The composition comprising at least one gluten peptide may also be a composition comprising the epitopes of the peptide pools provided in the Examples. In embodiments of any one of the compositions provided, the peptides are in equimolar amounts.

In some embodiments, the determining step comprises contacting (e.g., contacting ex vivo) the sample comprising the T cell with the composition comprising at least one gluten peptide; and measuring the level of IP-10 in the sample comprising the T cell after the contacting. Methods for measuring the level of IP-10 are described herein.

In some embodiments, any one of the methods further comprises comparing the level of IP-10 in the sample with a control level of IP-10. The comparing may be accomplished with the assistance of a software program on a computer. In some embodiments, the comparing comprises a statistical analysis, such as a paired T-test.

In some embodiments, assessing comprises identifying the subject as having or at risk of having Celiac disease if the IP-10 level is elevated compared to a control level of IP-10; or not having or not at risk of having Celiac disease if the IP-10 level is reduced compared to the control level of IP-10 or the same as the control level of IP-10. Control levels are further described herein. In some embodiments, assessing comprises identifying the subject as having or at risk of having Celiac disease if the IP-10 level is at least 100 pg/mL above a control level of IP-10; or not having or not at risk of having Celiac disease if the IP-10 level is less than 100 pg/mL above a control level of IP-10 (e.g. whole blood incubated with a composition described herein comprising at least one gluten peptide in phosphate buffered saline (PBS) compared to PBS alone). In some embodiments, assessing comprises identifying the subject as having or at risk of having Celiac disease if the IP-10 level is at least 96 pg/mL or 116 pg/mL above a control level of IP-10; or not having or not at risk of having Celiac disease if the IP-10 level is less than 96 pg/mL or 116 pg/mL above a control level of IP-10 (e.g. whole blood incubated with a composition described herein comprising at least one gluten peptide in phosphate buffered saline (PBS) compared to PBS alone). In some embodiments, assessing comprises identifying the subject as having or at risk of having Celiac disease if the IP-10 level is at least 25% greater a control level of IP-10; or not having or not at risk of having Celiac disease if the IP-10 level is less than 25% greater than a control level of IP-10. In some embodiments, assessing comprises identifying the subject as having or at risk of having Celiac disease if the IP-10 level is at least 25% greater than and at least 100 pg/mL above a control level of IP-10; or not having or not at risk of having Celiac disease if the IP-10 level is less than 25% greater than and is less than 100 pg/mL above a control level of IP-10. In some embodiments, assessing comprises identifying the subject as having or at risk of having Celiac disease if the IP-10 level is at least 25% greater than and at least 96 pg/mL or 116 pg/mL above a control level of IP-10; or not having or not at risk of having Celiac disease if the IP-10 level is less than 25% greater than and is less than 96 pg/mL or 116 pg/mL above a control level of IP-10.

In some embodiments, assessing comprises identifying the subject as having or at risk of having Celiac disease if the IP-10 level is at least 100 pg/mL above a control level of IP-10 and a stimulation index that is greater than 1.25; or not having or not at risk of having Celiac disease if the IP-10 level is less than 100 pg/mL above a control level of IP-10 and a stimulation index that is less than or equal to 1.25. In some embodiments, assessing comprises identifying the subject as having or at risk of having Celiac disease if the IP-10 level is at least 96 pg/mL or 116 pg/mL above a control level of IP-10 and a stimulation index that is greater than 1.25; or not having or not at risk of having Celiac disease if the IP-10 level is less than 96 pg/mL or 116 pg/mL above a control level of IP-10 and a stimulation index that is less than or equal to 1.25.

In some embodiments of any one of the methods provided herein, the method further comprising treating or suggesting a treatment if the subject is identified as having or likely of having celiac disease. In some embodiments of any one of the methods provided herein, the method further comprises recommending a gluten-free diet and/or providing information in regard thereto to the subject. In some embodiments of any one of the methods provided herein, the method further comprises administering a treatment, or providing information in regard thereto, to the subject. Suitable treatments are described herein. In some embodiments, the treatment is a composition comprising a gluten peptide as described herein. In some embodiments, the treatment comprises a gluten-free diet.

In some embodiments, any one of the methods described herein further comprises recording whether or not the subject has celiac disease based on the assessing. In some embodiments, any one of the methods described herein further comprises transmitting, such as to a database, whether or not the subject has celiac disease based on the assessing. The transmitting may be accomplished, e.g., via a computer or network of computers.

### IP-10 and Measuring IP-10 Levels

IFN-γ inducible protein-10 (IP-10, also referred to as C—X—C motif chemokine 10, CXCL10, small-inducible cytokine B10, SCYB10, C7, IFI10, crg-2, gIP-10, or mob-1) is a protein that in humans is encoded by the CXCL10 gene. IP-10 is a small cytokine belonging to the CXC chemokine family and binds to the chemokine receptor CXCR3. The Genbank ID number for the human CXCL10 gene is 3627. Exemplary mRNA sequences and protein sequences for IP-10 are shown below.

>gi|323422857|ref|NM_001565.3
|Homo sapiens chemokine (C—X—C motif)
ligand 10 (CXCL10), mRNA

(SEQ ID NO: 66)

CTTTGCAGATAAATATGGCACACTAGCCCCACGTTTTCTGAGACATTCCT
CAATTGCTTAGACATATTCTGAGCCTACAGCAGAGGAACCTCCAGTCTCA
GCACCATGAATCAAACTGCCATTCTGATTTGCTGCCTTATCTTTCTGACT
CTAAGTGGCATTCAAGGAGTACCTCTCTCTAGAACTGTACGCTGTACCTG
CATCAGCATTAGTAATCAACCTGTTAATCCAAGGTCTTTAGAAAAACTTG
AAATTATTCCTGCAAGCCAATTTTGTCCACGTGTTGAGATCATTGCTACA
ATGAAAAAGAAGGGTGAGAAGAGATGTCTGAATCCAGAATCGAAGGCCAT
CAAGAATTTACTGAAAGCAGTTAGCAAGGAAAGGTCTAAAAGATCTCCTT
AAAACCAGAGGGGAGCAAAATCGATGCAGTGCTTCCAAGGATGGACCACA
CAGAGGCTGCCTCTCCCATCACTTCCCTACATGGAGTATATGTCAAGCCA
TAATTGTTCTTAGTTTGCAGTTACACTAAAAGGTGACCAATGATGGTCAC
CAAATCAGCTGCTACTACTCCTGTAGGAAGGTTAATGTTCATCATCCTAA
GCTATTCAGTAATAACTCTACCCTGGCACTATAATGTAAGCTCTACTGAG
GTGCTATGTTCTTAGTGGATGTTCTGACCCTGCTTCAAATATTTCCCTCA
CCTTTCCCATCTTCCAAGGGTACTAAGGAATCTTTCTGCTTTGGGGTTTA
TCAGAATTCTCAGAATCTCAAATAACTAAAAGGTATGCAATCAAATCTGC
TTTTTAAAGAATGCTCTTTACTTCATGGACTTCCACTGCCATCCTCCCAA
GGGGCCCAAATTCTTTCAGTGGCTACCTACATACAATTCCAAACACATAC
AGGAAGGTAGAAATATCTGAAAATGTATGTGTAAGTATTCTTATTTAATG
AAAGACTGTACAAAGTAGAAGTCTTAGATGTATATATTTCCTATATTGTT
TTCAGTGTACATGGAATAACATGTAATTAAGTACTATGTATCAATGAGTA
ACAGGAAAATTTTAAAAATACAGATAGATATATGCTCTGCATGTTACATA
AGATAAATGTGCTGAATGGTTTTCAAAATAAAAATGAGGTACTCTCCTGG
AAATATTAAGAAAGACTATCTAAATGTTGAAAGATCAAAAGGTTAATAAA
GTAATTATAACTAAGAAAAAAAAAAAA
>gi|149999382|ref|NP_001556.2|
C—X—C motif chemokine 10 precursor
[Homo sapiens]

(SEQ ID NO: 67)

MNQTAILICCLIFLTLSGIQGVPLSRTVRCTCISISNQPVNPRSLEKLEI
IPASQFCPRVEIIATMKKKGEKRCLNPESKAIKNLLKAVSKERSKRSP
>gi|149999382:22-98 C—X—C motif chemokine
10 mature protein [Homo sapiens]

(SEQ ID NO: 68)

VPLSRTVRCTCISISNQPVNPRSLEKLEIIPASQFCPRVEIIATMKKKGE
KRCLNPESKAIKNLLKAVSKERSKRSP

As described herein, levels of IP-10 from a sample comprising a lymphocyte, e.g., a whole blood sample or a peripheral blood mononuclear cell sample, were found to be highly elevated in blood samples from subjects having Celiac disease, even before undergoing a gluten challenge. Accordingly, aspects of the disclosure relate to methods that comprise determining or measuring a level of IP-10 in a sample comprising a T cell from a subject, such as a subject suspected of having Celiac disease. Such methods are described herein.

Measuring an IP-10 level can be accomplished using any assay known in the art (see, e.g., Molecular Cloning: A Laboratory Manual, M. Green and J. Sambrook, Fourth Edition, Cold Spring Harbor Laboratory Press, Cold Spring Harbor, N.Y., 2012, Current Protocols in Molecular Biology, F. M. Ausubel, et al., eds., John Wiley & Sons, Inc., New York). Levels of IP-10 include levels of IP-10 mRNA and/or levels of IP-10 protein. In a preferred embodiment, levels of IP-10 are protein levels.

Assays for detecting IP-10 mRNA include, but are not limited to, Northern blot analysis, RT-PCR, sequencing technology, RNA in situ hybridization (using e.g., DNA or RNA probes to hybridize to RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer) with addressable locations, such as an Affymetrix microarray (Affymetrix®, Santa Clara, Calif.)). Methods for designing nucleic acid binding partners, such as probes, are well known in the art. In some embodiments, the nucleic acid binding partners bind to a part of or an entire nucleic acid sequence of IP-10, such as a sequence provided herein.

Assays for detecting protein levels include, but are not limited to, immunoassays (also referred to herein as immune-based or immuno-based assays, e.g., Western blot and ELISA), Mass spectrometry, and multiplex bead-based assays. Binding partners for protein detection can be designed using methods known in the art and as described herein. In some embodiments, the IP-10 protein binding partners, e.g., anti-IP-10 antibodies, bind to a part of or an entire amino acid sequence of IP-10, such as an IP-10 protein sequence provided herein. Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

In some embodiments, measuring a level of IP-10 comprises a multiplex bead-based assay. An exemplary multiplex bead-based assay involves use of magnetic beads that are internally dyed with fluorescent dyes to produce a specific spectral address. Binding partners (e.g., antibodies) are conjugated to the surface of beads to capture IP-10. The sample is loaded into a 96-well plate containing the beads and the sample is incubated to allow binding of IP-10 to the beads. A second biotinylated binding partner for IP-10 is added after the IP-10 binds to the beads. A streptavidin-conjugated detectable label is then bound to the biotin. Light emitting diodes are used to illuminate the samples, causing the fluorescent dyes in the beads to fluoresce, as well as the detectable label to fluoresce. The concentration of IP-10 is then determined based on the level of fluorescence. An exemplary system for running a multiplex bead-based assay is the MAGPIX® system available from Luminex® Corporation (see, e.g., U.S. Pat. No. 8,031,918, U.S. Pat. No. 8,296,088, U.S. Pat. No. 8,274,656, U.S. Pat. No. 8,532,351, U.S. Pat. No. 8,542,897, U.S. Pat. No. 6,514,295, U.S. Pat. No. 6,599,331, U.S. Pat. No. 6,632,526, U.S. Pat. No. 6,929,859, U.S. Pat. No. 7,445,844, U.S. Pat. No. 7,718,262, U.S. Pat. No. 8,283,037, and U.S. Pat. No. 8,568,881, all of which are incorporated by reference herein, and in particular the systems provided herein).

In some embodiments, measuring a level of IP-10 comprises an enzyme-linked immunosorbent assay (ELISA). ELISA is well known in the art (see, e.g., U.S. Pat. Nos. 5,939,281, 6,410,252, and Lequin R (2005). “Enzyme immunoassay (EIA)/enzyme-linked immunosorbent assay (ELISA)”. Clin. Chem. 51(12): 2415-8).

An exemplary ELISA involves at least one binding partner, e.g., an antibody or antigen-binding fragment thereof, with specificity for a particular antigen, such as IP-10. The sample with an unknown amount of antigen can be immobilized on a solid support (e.g., a polystyrene microtiter plate) either non-specifically (via adsorption to the surface) or specifically (via capture by another binding partner specific to the same antigen, as in a “sandwich” ELISA). After the antigen is immobilized, the binding partner for IP-10 is added, forming a complex with the immobilized IP-10. The binding partner can be attached to a detectable label as described herein (e.g., a fluorophor or an enzyme), or can itself be detected by an agent that recognizes the IP-10 binding partner that is attached to a detectable label as described herein (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

In some embodiments, a level of IP-10 is measured using an ELISA similar to the QuantiFERON®-TB Gold IT test (Cellestis Inc., Valencia, Calif.) for detecting mycobacterium, except wherein the TB antigen is replaced with at least one gluten peptide as described herein and IP-10 is detected in place of IFN-γ. The ELISA in the context of TB antigen has been described (see, e.g., U.S. Pat. Nos. 5,494,799, 5,334,504, and 7,608,382). As an exemplary method, at least one gluten peptide as defined herein is dried onto the inner wall of a blood collection tube. A negative control tube containing no antigen is provided. A positive control tube containing a mitogen is also provided. Blood from a subject is drawn into each of the three tubes. Each tube is agitated to ensure mixing. The tubes are then incubated at 37 degrees Celsius, preferably immediately after blood draw or at least within about 16 hours of collection. After incubation, the cells are separated from the plasma by centrifugation. The plasma is then loaded into an ELISA plate for detection of levels of IP-10 present in the plasma. An standard ELISA assay as described above can then be used to detect the levels of IP-10 present in each plasma sample.

In some embodiments, the level of IP-10 detected using any one of the methods above or any other appropriate method is then compared to a control level of IP-10 as described herein. In some embodiments, the control level is measured using any one of the methods above or any other as appropriate. In some embodiments, the same method is used to detect the level of the IP-10 in the sample of the subject and in the control level of IP-10.

### Samples

Samples, as used herein, refer to biological samples taken or derived from a subject, e.g., a subject having or suspected of having Celiac disease. Examples of samples include tissue samples or fluid samples. Examples of fluid samples are blood, plasma, and serum. In some embodiments, the sample comprises a T cell. In some embodiments, the sample comprises a T cell and a leukocyte, such as a monocyte or granulocyte. In some embodiments, the sample comprises a T cell and monocyte or granulocyte. In some embodiments, the sample comprises a T cell, a monocyte and a granulocyte. Different types of leukocytes can be identified using methods known in the art, e.g., using a Hematoxylin and eosin stain and/or antibodies specific for different types of leukocytes. In some embodiments, the sample comprises whole blood or peripheral blood mononuclear cells (PBMCs). Whole blood includes blood cells (such as erythrocytes, leukocytes, and platelets) and plasma, and may optionally include additives such as anti-coagulants. PBMCs include singly-nucleated blood cells (such as lymphocytes, monocytes, and macrophages) isolated from whole blood, e.g., using Ficoll or other methods known in the art. T cells include CD8+ and/or CD4+ T cells. The T cell may be, e.g., a gluten-reactive CD4+ T cell.

In some embodiments, any one of the methods described herein comprises obtaining the sample from the subject. In some embodiments in any one of the methods provided, a first and second sample are contemplated. “First” and “second” are not meant to imply an order of use or an order in which the samples are obtained, unless specifically stated otherwise. In some embodiments in any one of the methods provided, the second sample is a control sample to be used to obtain a control IP-10 level (controls and control levels are discussed herein). In some embodiments in any one of the methods provided, the first sample and/or second sample are obtained from the subject prior to, during, or after a gluten challenge as described herein. In some embodiments in any one of the methods provided, the first sample is obtained from the subject after a gluten challenge. In some embodiments in any one of the methods provided, the second sample is obtained from the subject prior to a gluten challenge. In some embodiments in any one of the methods provided, the first sample and/or second sample are obtained from the subject prior to, during, or after administration of a composition comprising the first, second, and third peptides as described herein (e.g., comprising 10 micrograms of the first peptide and an equimolar amount of each of the second and third peptides; 15 micrograms of the first peptide and an equimolar amount of each of the second and third peptides; 20 micrograms of the first peptide and an equimolar amount of each of the second and third peptides; or 50 micrograms of the first peptide and an equimolar amount of each of the second and third peptides). In some embodiments, the composition is administered intradermally. In some embodiments, the composition is administered to the subject once. In some embodiments in any one of the methods provided, the first sample is obtained from the subject after the composition is administered. In some embodiments in any one of the methods provided, the second sample is obtained from the subject prior to the composition being administered. Additional samples, e.g., third, fourth, fifth, etc., are also contemplated if additional measurements of IP-10 levels are desired.

### Subjects

A subject may include any subject that is suspected of having Celiac disease. Preferably, the subject is a human. In some embodiments, the subject has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). In some embodiments, the subject is HLA-DQ2.5 positive (i.e., has both susceptibility alleles DQA1*05 and DQB1*02). In some embodiments, the subject is HLA-DQ2.2 positive (i.e., has both susceptibility alleles DQA1*02 and DQB1*02). In some embodiments, the subject is HLA-DQ8 positive (i.e., has both susceptibility alleles DQA1*03 and DQB1*0302). In some embodiments, the subject is HLA-DQ2.2 positive and HLA-DQ2.5 positive. In some embodiments, the subject is HLA-DQ8 positive and HLA-DQ2.5 positive. In some embodiments, the subject is HLA-DQ2.2 positive and HLA-DQ8 positive. In some embodiments, a subject may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes. In some embodiments of any one of the methods provided herein, the subject is on a gluten-free diet. In some embodiments of any one of the methods provided herein, the subject is on a diet that contains gluten. In some embodiments of any one of the methods provided, the subject has consumed gluten within one week of the sample being obtained for testing.

In some embodiments of any one of the methods provided herein, the subject has a level of IFN-γ that is reduced or the same as a control level of IFN-γ. In some embodiments of any one of the methods provided herein, the subject's level of IFN-γ is such that a clinician may expect little to no risk of Celiac disease for the subject. In some embodiments, the level of IFN-γ is such that a clinician would expect additional testing to be needed for a more assured diagnosis. Based on the sensitivity provided by measuring IP-10, any one of the methods provided herein may reduce or eliminate the need for other testing. A level of IFN-γ may be measured using any method known in the art or described herein (e.g., ELISA, ELISpot, or multiplex bead-based assay). The level may be a RNA level or a protein level. Exemplary RNA and protein sequences of IFN-γ are provided below.

>gi|56786137|ref|NM_000619.2
|Homo sapiens interferon, gamma (IFNG), mRNA

(SEQ ID NO: 69)

CACATTGTTCTGATCATCTGAAGATCAGCTATTAGAAGAGAAAGATCAGT
TAAGTCCTTTGGACCTGATCAGCTTGATACAAGAACTACTGATTTCAACT
TCTTTGGCTTAATTCTCTCGGAAACGATGAAATATACAAGTTATATCTTG
GCTTTTCAGCTCTGCATCGTTTTGGGTTCTCTTGGCTGTTACTGCCAGGA
CCCATATGTAAAAGAAGCAGAAAACCTTAAGAAATATTTTAATGCAGGTC
ATTCAGATGTAGCGGATAATGGAACTCTTTTCTTAGGCATTTTGAAGAAT
TGGAAAGAGGAGAGTGACAGAAAAATAATGCAGAGCCAAATTGTCTCCTT
TTACTTCAAACTTTTTAAAAACTTTAAAGATGACCAGAGCATCCAAAAGA
GTGTGGAGACCATCAAGGAAGACATGAATGTCAAGTTTTTCAATAGCAAC
AAAAAGAAACGAGATGACTTCGAAAAGCTGACTAATTATTCGGTAACTGA
CTTGAATGTCCAACGCAAAGCAATACATGAACTCATCCAAGTGATGGCTG
AACTGTCGCCAGCAGCTAAAACAGGGAAGCGAAAAAGGAGTCAGATGCTG
TTTCGAGGTCGAAGAGCATCCCAGTAATGGTTGTCCTGCCTGCAATATTT
GAATTTTAAATCTAAATCTATTTATTAATATTTAACATTATTTATATGGG
GAATATATTTTTAGACTCATCAATCAAATAAGTATTTATAATAGCAACTT
TTGTGTAATGAAAATGAATATCTATTAATATATGTATTATTTATAATTCC
TATATCCTGTGACTGTCTCACTTAATCCTTTGTTTTCTGACTAATTAGGC
AAGGCTATGTGATTACAAGGCTTTATCTCAGGGGCCAACTAGGCAGCCAA
CCTAAGCAAGATCCCATGGGTTGTGTGTTTATTTCACTTGATGATACAAT
GAACACTTATAAGTGAAGTGATACTATCCAGTTACTGCCGGTTTGAAAAT
ATGCCTGCAATCTGAGCCAGTGCTTTAATGGCATGTCAGACAGAACTTGA
ATGTGTCAGGTGACCCTGATGAAAACATAGCATCTCAGGAGATTTCATGC
CTGGTGCTTCCAAATATTGTTGACAACTGTGACTGTACCCAAATGGAAAG
TAACTCATTTGTTAAAATTATCAATATCTAATATATATGAATAAAGTGTA
AGTTCACAACAAAAAAAAAAAAAAAAAAAAAAAAAAAAAA
>gi|56786138|ref|NP_000610.2|
interferon gamma precursor [Homo sapiens]

(SEQ ID NO: 70)

MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ
>gi|56786138: 24-166 interferon gamma mature
protein [Homo sapiens]

(SEQ ID NO: 71)

QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQMLFRGRRASQ

In some embodiments, a control level is a level of IFN-γ in a sample from a control subject (or subjects). Control subjects are described herein. In some embodiments, the control level is a pre-determined threshold. In some embodiments, the control level of IFN-γ is 7.2 pg/mL. In some embodiments, a control level is a level of IFN-γ in a second sample from the same subject from which the first sample was obtained (e.g., a first and second sample may be obtained from the same subject and the comparison between the first and second sample is used to determine if the subject has or is at risk of having Celiac disease). In some embodiments, the first sample and/or second sample is obtained from the subject prior to, during, or after a gluten challenge as described herein.

### Controls and Control Levels

In some embodiments, methods provided herein comprise measuring a level of IP-10 in a sample (e.g., a first sample) and then comparing that level to one or more control levels of IP-10.

In some embodiments, a control level is a level of IP-10 in a sample from a control subject (or subjects). In some embodiments, a control subject has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease. In some embodiments, a control subject does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments, a control subject is a healthy individual not having or suspected of having Celiac disease. In some embodiments, the control level is a pre-determined threshold. In some embodiments, the control level of IP-10 is 100 pg/mL. In some embodiments, the control level of IP-10 is 96 pg/mL or 116 pg/mL. In some embodiments, a control level is a pre-determined level from a control subject or subjects, such that the control level need not be measured every time the methods described herein are performed.

In some embodiments of any one of the methods provided, a control level is a level of IP-10 in a second sample from the same subject from which the first sample was obtained (e.g., a first and second sample may be obtained from the same subject and the comparison between the first and second sample is used to determine if the subject has or is at risk of having Celiac disease). In some embodiments of any one of the methods provided, the first sample and/or second sample is obtained from the subject prior to, during, or after a gluten challenge as described herein. In some embodiments of any one of the methods provided, the first sample is obtained from the subject after a gluten challenge. In some embodiments of any one of the methods provided, the second sample is obtained from the subject prior to a gluten challenge. In some embodiments of any one of the methods provided, a control level is a level of IP-10 is a negative control level of IP-10. Exemplary negative controls include, but are not limited to, a level of IP-10 in a sample that has been contacted with a non-T cell-activating peptide (e.g., a peptide not recognized by T cells present in a sample from a subject), such as a non-CD4+-T cell-activating peptide, or a T cell response in sample that has not been contacted with a T cell-activating peptide (e.g., contacting the sample with a saline solution or cell culture medium containing no peptides), such as a CD4+ T cell-activating peptide. Additional control samples, e.g., third, fourth, fifth, etc., are also contemplated if additional measurements of IP-10 levels are desired.

### Gluten Peptides and Compositions Containing Gluten Peptides

As used herein the term “gluten peptide” includes any peptide comprising a sequence derived from, or encompassed within, one or more of gluten proteins alpha (a), beta (13), γ (γ) and omega (w) gliadins, and low and high molecular weight (LMW and HMW) glutenins in wheat, B, C and D hordeins in barley, β, γ and omega secalins in rye, and optionally avenins in oats, including deamidated variants thereof containing one or more glutamine to glutamate substitutions. In some embodiments, the gluten peptide(s) stimulate a CD4+ T cell specific response.

A gluten peptide may comprise or consist of one or more sequences of epitopes known to be recognized by a CD4+ T cell in a subject with Celiac disease, e.g., sequences encompassing PELP (SEQ ID NO: 72), PELPY (SEQ ID NO: 73), QPELPYP (SEQ ID NO: 74), PQPELPY (SEQ ID NO: 75), FPQPELP (SEQ ID NO: 76), PELPYPQ (SEQ ID NO: 77), FPQPELPYP (SEQ ID NO: 78), PYPQPELPY (SEQ ID NO: 19), PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), PQPELPYPQ (SEQ ID NO: 2), FRPEQPYPQ (SEQ ID NO: 79), PQQSFPEQQ (SEQ ID NO: 80), IQPEQPAQL (SEQ ID NO: 81), QQPEQPYPQ (SEQ ID NO: 82), SQPEQEFPQ (SEQ ID NO: 83), PQPEQEFPQ (SEQ ID NO: 84), QQPEQPFPQ (SEQ ID NO: 85), PQPEQPFCQ (SEQ ID NO: 86), QQPFPEQPQ (SEQ ID NO: 87), PFPQPEQPF (SEQ ID NO: 3), PQPEQPFPW (SEQ ID NO: 4), PFSEQEQPV (SEQ ID NO: 18), FSQQQESPF (SEQ ID NO: 88), PFPQPEQPF (SEQ ID NO: 3), PQPEQPFPQ (SEQ ID NO: 89), PIPEQPQPY (SEQ ID NO: 6), PFPQPEQPF (SEQ ID NO: 3), PQPEQPFPQ (SEQ ID NO: 90), PYPEQEEPF (SEQ ID NO: 91), PYPEQEQPF (SEQ ID NO: 92), PFSEQEQPV (SEQ ID NO: 18), EGSFQPSQE (SEQ ID NO: 15), EQPQQPFPQ (SEQ ID NO: 93), EQPQQPYPE (SEQ ID NO: 94), QQGYYPTSPQ (SEQ ID NO: 95), EGSFQPSQE (SEQ ID NO: 15), PQQSFPEQE (SEQ ID NO: 96), or QGYYPTSPQ (SEQ ID NO: 16) (see, e.g., Sollid L M, Qiao S W, Anderson R P, Gianfrani C, Koning F. Nomenclature and listing of celiac disease relevant gluten epitopes recognized by CD4+ T cells. Immunogenetics. 2012; 64:455-60; PCT Publication Nos.: WO/2001/025793, WO/2003/104273, WO/2005/105129, and WO/2010/060155). Preferably, in some embodiments, the gluten peptides that comprise sequences of epitopes of less than 6 amino acids also comprise additional amino acids flanking either or both sides of the epitope. Preferably, in some embodiments, the gluten peptides are at least 8 or 9 amino acids in length.

In some embodiments of any one of the methods or kits provided, a gluten peptide may comprise or consist of one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23). In some embodiments of any one of the methods or kits provided, a gluten peptide may comprise or consist of the T cell epitope sequences PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6) and at least one further amino acid sequence selected from PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23). In some embodiments of any one of the methods or kits provided, a gluten peptide may comprise or consist of the T cell epitope sequences EQPFPEQPI (SEQ ID NO: 20), PFPEQPIPE (SEQ ID NO: 21), EQPIPEQPQ (SEQ ID NO: 5), and PIPEQPQPY (SEQ ID NO: 6). In some embodiments of any one of the methods or kits provided, a gluten peptide may include one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), PFPQPEQPIP (SEQ ID NO: 62), EQPIPVQPE (SEQ ID NO: 9), PFPQPEQPTPI (SEQ ID NO: 63), EQPTPIQPE (SEQ ID NO: 12), PQPEQPFPL (SEQ ID NO: 13), EQPFPLQPE (SEQ ID NO: 14), PFPQPEQPF (SEQ ID NO: 3), PQPEQPFSQ (SEQ ID NO: 40), PYPEQPQPF (SEQ ID NO: 22), PFPEQPEQIIP (SEQ ID NO: 64), EGSFQPSQE (SEQ ID NO: 15), QGYYPTSPQ (SEQ ID NO: 16), EQPEQPFPEQPQ (SEQ ID NO: 65), PFSEQEQPV (SEQ ID NO: 18), EQPFPEQPI (SEQ ID NO: 20), PIPEQPQPY (SEQ ID NO: 6), PQPELPYPQ (SEQ ID NO: 2), PYPQPELPY (SEQ ID NO: 19), PFPQPELPY (SEQ ID NO: 1), and PQPELPYPY (SEQ ID NO: 43). Preferably, in some embodiments, the gluten peptides are at least 8 or 9 amino acids in length.

In some embodiments of any one of the methods or kits provided, the gluten peptide is selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 40);

(h) an eighth peptide comprising the amino acid sequence PYPEQPQPF (SEQ ID NO: 22);

(i) a ninth peptide comprising the amino acid sequence PFPEQPEQI (SEQ ID NO: 42);

(j) a tenth peptide comprising the amino acid sequence EGSFQPSQE (SEQ ID NO: 15);

(k) an eleventh peptide comprising the amino acid sequence QGYYPTSPQ (SEQ ID NO: 16);

(l) a twelfth peptide comprising the amino acid sequence EQPEQPFPE (SEQ ID NO: 17) and the amino acid sequence EQPFPEQPQ (SEQ ID NO: 41);

(m) a thirteenth peptide comprising the amino acid sequence PFSEQEQPV (SEQ ID NO: 18);

(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 19);

(p) a sixteenth peptide comprising the amino acid sequence PFPQPELPY (SEQ ID NO: 1) and the amino acid sequence PQPELPYPY (SEQ ID NO: 43);

(q) a seventeenth peptide comprising the amino acid sequence EQPFPEQPI (SEQ ID NO: 20) and the amino acid sequence PFPEQPIPE (SEQ ID NO: 21); and

(r) an eighteenth peptide comprising the amino acid sequence PQPYPEQPQ (SEQ ID NO: 23) and the amino acid sequence PYPEQPQPF (SEQ ID NO: 22). In some embodiments, any one or more of the peptides herein comprises an N-terminal pyroglutamate and/or a C-terminal amide group.)

In some embodiments of any one of the methods or kits provided, a gluten peptide may include one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23).

In some embodiments of any one of the methods or kits provided, the gluten peptide is selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 15);

(k) a eleventh peptide comprising the amino acid sequence QGYYPTSPQ (SEQ ID NO: 16);

(l) a twelfth peptide comprising the amino acid sequence EQPEQPFPE (SEQ ID NO: 17);

(m) a thirteenth peptide comprising the amino acid sequence PFSEQEQPV (SEQ ID NO: 18);

(n) a fourteenth peptide comprising the amino acid sequence PYPQPELPY (SEQ ID NO: 19);

(o) a fifteenth peptide comprising the amino acid sequence EQPFPEQPI (SEQ ID NO: 20) and the amino acid sequence PFPEQPIPE (SEQ ID NO: 21); and

(p) a sixteenth peptide comprising the amino acid sequence PYPEQPQPF (SEQ ID NO: 22) and the amino acid sequence PQPYPEQPQ (SEQ ID NO: 23). In some embodiments, any one or more of the peptides herein comprises an N-terminal pyroglutamate and/or a C-terminal amide group.

In some embodiments, a gluten peptide may comprise or consist of one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), PFPQPEQPIP (SEQ ID NO: 62), EQPIPVQPE (SEQ ID NO: 9), PFPQPEQPTPI (SEQ ID NO: 63), EQPTPIQPE (SEQ ID NO: 12), PQPEQPFPL (SEQ ID NO: 13), EQPFPLQPE (SEQ ID NO: 14), PFPQPEQPF (SEQ ID NO: 3), PQPEQPFSQ (SEQ ID NO: 40), PYPEQPQPF (SEQ ID NO: 22), PFPEQPEQIIP (SEQ ID NO: 64), EGSFQPSQE (SEQ 20 ID NO: 15), QGYYPTSPQ (SEQ ID NO: 16), EQPEQPFPEQPQ (SEQ ID NO: 65), PFSEQEQPV (SEQ ID NO: 18), EQPFPEQPI (SEQ ID NO: 20), PIPEQPQPY (SEQ ID NO: 6), PQPELPYPQ (SEQ ID NO: 2), PYPQPELPY (SEQ ID NO: 19), PFPQPELPY (SEQ ID NO: 1), and PQPELPYPY (SEQ ID NO: 43).

In some embodiments, the gluten peptide is selected from:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 63) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 40);

(viii) an eighth peptide comprising the amino acid sequence PYPEQPQPF (SEQ ID NO: 22);

(ix) a ninth peptide comprising the amino acid sequence PFPEQPEQIIP (SEQ ID S10 NO: 64);

(x) a tenth peptide comprising the amino acid sequence EGSFQPSQE (SEQ ID NO: 15);

(xi) an eleventh peptide comprising the amino acid sequence QGYYPTSPQ (SEQ ID NO: 16);

(xii) a twelfth peptide comprising the amino acid sequence EQPEQPFPEQPQ (SEQ ID NO: 65);

(xiii) a thirteenth peptide comprising the amino acid sequence PFSEQEQPV (SEQ ID NO: 18);

(xiv) a fourteenth peptide comprising the amino acid sequence EQPFPEQPI (SEQ ID NO: 20) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 19);

(xvi) a sixteenth peptide comprising the amino acid sequence PFPQPELPY (SEQ ID NO: 1) and the amino acid sequence PQPELPYPY (SEQ ID NO: 43); and

(xvii) a seventeenth peptide comprising the amino acid sequence EQPFPEQPI (SEQ ID NO: 20). In some embodiments, any one of the peptides herein comprises an N-terminal pyroglutamate and/or a C-terminal amide group.

Exemplary gluten peptides and method for synthesizing or obtaining such peptides are known in the art and described herein (see, e.g., PCT Publication Nos.: WO/2001/025793, WO/2003/104273, WO/2005/105129, and WO/2010/060155, which are incorporated herein by reference in their entirety, including specifically the aforementioned peptides and methods).

A gluten peptide can be recombinantly and/or synthetically produced. In some embodiments, a gluten peptide is chemically synthesized, e.g., using a method known in the art. Non-limiting examples of peptide synthesis include liquid-phase synthesis and solid-phase synthesis. In some embodiments, a gluten peptide is produced by enzymatic digestion, e.g., by enzymatic digestion of a larger polypeptide into short peptides.

In some embodiments, one or more glutamate residues of a gluten peptide may be generated by tissue transglutaminase (tTG) deamidation activity upon one or more glutamine residues of the gluten peptide. This deamidation of glutamine to glutamate can cause the generation of gluten peptides that can bind to HLA-DQ2 or -DQ8 molecules with high affinity. This reaction may occur in vitro by contacting the gluten peptide composition with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 97) could become PFPQPELPY (SEQ ID NO: 1) after processing by tTG. Conservative substitution of E with D is also contemplated herein in any one of the peptides provided herein (e.g., PFPQPELPY (SEQ ID NO: 1) could become PFPQPDLPY (SEQ ID NO: 98)). Exemplary peptides including an E to D substitution include peptides comprising or consisting of one or more of the sequences selected from PFPQPDLPY (SEQ ID NO: 98), PQPDLPYPQ (SEQ ID NO: 99), PFPQPDQPF (SEQ ID NO: 100), PQPDQPFPW (SEQ ID NO: 101), PIPDQPQPY (SEQ ID NO: 102), LQPFPQPDLPYPQPQ (SEQ ID NO: 103), QPFPQPDQPFPWQP (SEQ ID NO: 104), PQQPIPDQPQPYPQQ (SEQ ID NO: 105), PFPQPDQPIP (SEQ ID NO: 106), DQPIPVQPD (SEQ ID NO: 107), PFPQPDQPTPI (SEQ ID NO: 108), DQPTPIQPD (SEQ ID NO: 109), PQPDQPFPL (SEQ ID NO: 110), DQPFPLQPD (SEQ ID NO: 111), PFPQPDQPF (SEQ ID NO: 112), PQPDQPFSQ (SEQ ID NO: 113), PYPDQPQPF (SEQ ID NO: 114), PFPDQPDQIIP (SEQ ID NO: 115), DGSFQPSQD (SEQ ID NO: 116), DQPDQPFPDQPQ (SEQ ID NO: 117), PFSDQDQPV (SEQ ID NO: 118), DQPFPDQPI (SEQ ID NO: 119), PIPDQPQPY (SEQ ID NO: 120), PQPDLPYPQ (SEQ ID NO: 121), PYPQPDLPY (SEQ ID NO: 122), PFPQPDLPY (SEQ ID NO: 98), and PQPDLPYPY (SEQ ID NO: 123). Other exemplary peptides including an E to D substitution include peptides comprising or consisting of one or more of the sequences selected from PFPQPDLPY (SEQ ID NO: 98), PQPDLPYPQ (SEQ ID NO: 121), PFPQPDQPF (SEQ ID NO: 112), PQPDQPFPW (SEQ ID NO: 124), DQPIPDQPQ (SEQ ID NO: 146), PIPDQPQPY (SEQ ID NO: 125), PFPQPDQPI (SEQ ID NO: 126), PQPDQPIPV (SEQ ID NO: 127), DQPIPVQPE (SEQ ID NO: 128), PFPQPDQPT (SEQ ID NO: 129), PQPDQPTPI (SEQ ID NO: 130), DQPTPIQPD (SEQ ID NO: 131), PQPDQPFPL (SEQ ID NO: 132), DQPFPLQPD (SEQ ID NO: 133), PQPDQPFSQ (SEQ ID NO: 145), PYPDQPQPF (SEQ ID NO: 134), DGSFQPSQD (SEQ ID NO: 135), DQPQQPFPD (SEQ ID NO: 136), DQPDQPFPQ (SEQ ID NO: 137), DQPFPDQPQ (SEQ ID NO: 138), PFPDQPDQI (SEQ ID NO: 139), PFSDQDQPV (SEQ ID NO: 140), DQPFPDQPI (SEQ ID NO: 141), PFPDQPIPD (SEQ ID NO: 142), PYPQPDLPY (SEQ ID NO: 143), PQPDLPYPY (SEQ ID NO: 123), and PQPYPDQPQ (SEQ ID NO: 144). Such substituted peptides can be the gluten peptides of any one of the methods and compositions provided herein.

In some embodiments, it may be desirable to utilize the non-deamidated forms of such peptides, e.g., if the peptides are contained within a composition for administration to a subject where tissue transglutaminase will act in situ (see, e.g., Øyvind Molberg, Stephen McAdam, Knut E. A. Lundin, Christel Kristiansen, Helene Arentz-Hansen, Kjell Kett and Ludvig M. Sollid. T cells from celiac disease lesions recognize gliadin epitopes deamidated in situ by endogenous tissue transglutaminase. Eur. J. Immunol. 2001. 31: 1317-1323). Accordingly, gluten peptides that have not undergone deamidation are also contemplated herein (e.g., gluten peptides comprising or consisting of one or more of the sequences selected from: PQLP (SEQ ID NO: 147), PQLPY (SEQ ID NO: 148), QPQLPYP (SEQ ID NO: 149), PQPQLPY (SEQ ID NO: 150), FPQPQLP (SEQ ID NO: 151), PQLPYPQ (SEQ ID NO: 152), FPQPQLPYP (SEQ ID NO: 153), PYPQPQLPY (SEQ ID NO: 154), PFPQPQLPY (SEQ ID NO: 97), PQPQLPYPQ (SEQ ID NO: 155), PFPQPQQPF (SEQ ID NO: 156), PQPQQPFPW (SEQ ID NO: 157), PIPQQPQPY (SEQ ID NO: 158), LQPFPQPQLPYPQPQ (SEQ ID NO: 159), QPFPQPQQPFPWQP (SEQ ID NO: 160), PEQPIPQQPQPYPQQ (SEQ ID NO: 161), PQPQLPYPQ (SEQ ID NO: 162), FRPQQPYPQ (SEQ ID NO: 163), PQQSFPQQQ (SEQ ID NO: 164), IQPQQPAQL (SEQ ID NO: 165), QQPQQPYPQ (SEQ ID NO: 166), SQPQQQFPQ (SEQ ID NO: 167), PQPQQQFPQ (SEQ ID NO: 168), QQPQQPFPQ (SEQ ID NO: 169), PQPQQPFCQ (SEQ ID NO: 170), QQPFPQQPQ (SEQ ID NO: 171), PFPQPQQPF (SEQ ID NO: 172), PQPQQPFPW (SEQ ID NO: 173), PFSQQQQPV (SEQ ID NO: 174), FSQQQQSPF (SEQ ID NO: 175), PFPQPQQPF (SEQ ID NO: 172), PQPQQPFPQ (SEQ ID NO: 176), PIPQQPQPY (SEQ ID NO: 177), PFPQPQQPF (SEQ ID NO: 178), PQPQQPFPQ (SEQ ID NO: 179), PYPEQQEPF (SEQ ID NO: 180), PYPEQQQPF (SEQ ID NO: 181), PFSQQQQPV (SEQ ID NO: 182), QGSFQPSQQ (SEQ ID NO: 183), QQPQQPFPQ (SEQ ID NO: 184), QQPQQPYPQ (SEQ ID NO: 185), QQGYYPTSPQ (SEQ ID NO: 222), QGSFQPSQQ (SEQ ID NO: 186), PQQSFPQQQ (SEQ ID NO: 187), QGYYPTSPQ (SEQ ID NO: 16), LQPFPQPELPYPQPQ (SEQ ID NO: 45), QPFPQPQQPFPWQP (SEQ ID NO: 188), PQQPIPQQPQPYPQQ (SEQ ID NO: 189), PFPQPEQPIP (SEQ ID NO: 62), EQPIPVQPE (SEQ ID NO: 9), PFPQPEQPTPI (SEQ ID NO: 63), EQPTPIQPE (SEQ ID NO: 12), PQPEQPFPL (SEQ ID NO: 13), EQPFPLQPE (SEQ ID NO: 14), PFPQPEQPF (SEQ ID NO: 3), PQPEQPFSQ (SEQ ID NO: 40), PYPEQPQPF (SEQ ID NO: 22), PFPEQPEQIIP (SEQ ID NO: 64), EGSFQPSQE (SEQ ID NO: 15), EQPEQPFPEQPQ (SEQ ID NO: 65), PFSEQEQPV (SEQ ID NO: 18), EQPFPEQPI (SEQ ID NO: 20), PIPEQPQPY (SEQ ID NO: 6), PQPELPYPQ (SEQ ID NO: 2), PYPQPELPY (SEQ ID NO: 19), PFPQPELPY (SEQ ID NO: 1), PQPELPYPY (SEQ ID NO: 43), LQPFPQPQLPYPQPQ (SEQ ID NO: 190), QPFPQPQQPFPWQP (SEQ ID NO: 191), PQQPIPQQPQPYPQQ (SEQ ID NO: 192), QPFPQPQQPIPVQPQQS (SEQ ID NO: 193), QPFPQPQQPTPIQPQQP (SEQ ID NO: 194), QPFPQPQQPFPLQPQQP (SEQ ID NO: 195), QPFPQPQQPFSQQ (SEQ ID NO: 196), PQPYPQQPQPFPQQ (SEQ ID NO: 197), QPFPEQPQQIIPQQP (SEQ ID NO: 198), SGEGSFQPSQQNPQ (SEQ ID NO: 199), PQQPQQPFPQQPQQ (SEQ ID NO: 200), QPPFSQQQQPVLPQ (SEQ ID NO: 201), PQQPFPQQPIPQQPQPYP (SEQ ID NO: 202), QPYPQPQLPYPQPQ (SEQ ID NO: 203), and QPFPQPQLPYPYPQ (SEQ ID NO: 204) or gluten peptides comprising or consisting of one or more of the sequences selected from: PFPQPQLPY (SEQ ID NO: 97), PQPQLPYPQ (SEQ ID NO: 205), PFPQPQQPF (SEQ ID NO: 206), PQPQQPFPW (SEQ ID NO: 207), QQPIPQQPQ (SEQ ID NO: 208), PIPQQPQPY (SEQ ID NO: 209), PFPQPQQPI (SEQ ID NO: 210), PQPQQPIPV (SEQ ID NO: 211), QQPIPVQPQ (SEQ ID NO: 212), PFPQPQQPT (SEQ ID NO: 213), PQPQQPTPI (SEQ ID NO: 214), QQPTPIQPQ (SEQ ID NO: 215), PQPQQPFPL (SEQ ID NO: 216), QQPFPLQPQ (SEQ ID NO: 217), PQPQQPFSQ (SEQ ID NO: 218), PYPQQPQPF (SEQ ID NO: 219), QGSFQPSQQ (SEQ ID NO: 220), QGYYPTSPQ (SEQ ID NO: 16), QQPQQPFPQ (SEQ ID NO: 221), QQPQQPFPQ (SEQ ID NO: 221), QQPFPQQPQ (SEQ ID NO: 223), PFPQQPQQI (SEQ ID NO: 224), PFSQQQQPV (SEQ ID NO: 225), QQPFPQQPI (SEQ ID NO: 226), PFPQQPIPQ (SEQ ID NO: 227), PYPQPQLPY (SEQ ID NO: 228), PQPQLPYPY (SEQ ID NO: 229), and PQPYPQQPQ (SEQ ID NO: 230)).

A gluten peptide may also be an analog of any one of the peptides described herein. Preferably, in some embodiments the analog is recognized by a CD4⁺ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any one of the peptides provided, such variants can include conservative amino acid substitutions, e.g., E to D substitution.

In some embodiments, analogs may include one or more amino acid substitutions as shown in Table A (see, e.g., Anderson et al. Antagonists and non-toxic variants of the dominant wheat gliadin T cell epitope in coeliac disease. Gut. 2006 April; 55(4): 485-491; and PCT Publication WO2003104273, the contents of which are incorporated herein by reference, including the aforementioned analogs). The gluten peptides provided herein include analogs of FPQPELPYP (SEQ ID NO: 78) comprising one or more of the listed amino acid substitutions. In some embodiments, the analog is an analog of FPQPELPYP (SEQ ID NO: 78) comprising one of the amino acid substitutions provided in Table A below.

TABLE A
Exemplary substitutions in the core sequence FPQPELPY
(SEQ ID NO: 78) encompassed within the 17mer
QLQPFPQPELPYPQPQS (SEQ ID NO: 231)

| | Amino acid in epitope | | | | | | | | |
|---|---|---|---|---|---|---|---|---|---|
| | F | P | Q | P | E | L | P | Y | P |
| Exemplary | A, G, H, I, | A, F, I, M, | A, F, G, | — | D | M | S | I, S, | S, T, Y |
| Substitutions | L, M P, S, | S, T, V, | H, I, L, | | | | | V, W | |
| | T, W, Y | W, Y | M, S, T, V | | | | | | |

The length of the peptides may vary. In some embodiments of any one of the methods or kits provided, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments of any one of the methods or kits provided, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments of any one of the methods or kits provided, peptides are, e.g., 4-100, 4-50, 4-40, 4-30, or 4-20 amino acids in length. In some embodiments of any one of the methods or kits provided, peptides are 4-20, 5-20, 6-20, 7-20, 8-20, 9-20, 10-20, 11-20, 12-20, 13-20, 14-20, or 15-20 amino acids in length. In some embodiments of any one of the methods or kits provided, peptides are e.g., 5-30, 10-30, 15-30 or 20-30 amino acids in length. In some embodiments of any one of the methods or kits provided, peptides are 4-50, 5-50, 6-50, 7-50, 8-50, 9-50, 10-50, 11-50, 12-50, 13-50, 14-50, or 15-50 amino acids in length. In some embodiments of any one of the methods or kits provided, are 8-30 amino acids in length.

In some embodiments of any one of the methods provided herein, a composition comprising one or one or more gluten peptide(s) is contemplated. In some embodiments, the composition comprises at least one (e.g., 1, 2, 3, 4, 5, 7, 8, 9, 10, 11, 12, 13, 14, 15, or more) peptide, the at least one peptide comprising at least one (e.g., 1, 2, 3, 4, 5, 7, 8, 9, 10, 11, 12, 13, 14, 15, or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23).

In some embodiments, the composition comprises at least one (e.g., 1, 2, 3, 4, 5, 7, 8, 9, 10, 11, 12, 13, 14, 15, or more) peptide, the at least one peptide comprising at least one (e.g., 1, 2, 3, 4, 5, 7, 8, 9, 10, 11, 12, 13, 14, 15, or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), PFPQPEQPIP (SEQ ID NO: 62), EQPIPVQPE (SEQ ID NO: 9), PFPQPEQPTPI (SEQ ID NO: 63), EQPTPIQPE (SEQ ID NO: 12), PQPEQPFPL (SEQ ID NO: 13), EQPFPLQPE (SEQ ID NO: 14), PFPQPEQPF (SEQ ID NO: 3), PQPEQPFSQ (SEQ ID NO: 40), PYPEQPQPF (SEQ ID NO: 22), PFPEQPEQIIP (SEQ ID NO: 64), EGSFQPSQE (SEQ ID NO: 15), QGYYPTSPQ (SEQ ID NO: 16), EQPEQPFPEQPQ (SEQ ID NO: 65), PFSEQEQPV (SEQ ID NO: 18), EQPFPEQPI (SEQ ID NO: 20), PIPEQPQPY (SEQ ID NO: 6), PQPELPYPQ (SEQ ID NO: 2), PYPQPELPY (SEQ ID NO: 19), PFPQPELPY (SEQ ID NO: 1), PQPELPYPY (SEQ ID NO: 43), and EQPFPEQPI (SEQ ID NO: 20).

In some embodiments of any one of the methods provided herein, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 40);

(h) an eighth peptide comprising the amino acid sequence PYPEQPQPF (SEQ ID NO: 22);

(i) a ninth peptide comprising the amino acid sequence PFPEQPEQI (SEQ ID NO: 42);

(j) a tenth peptide comprising the amino acid sequence EGSFQPSQE (SEQ ID NO: 15);

(k) an eleventh peptide comprising the amino acid sequence QGYYPTSPQ (SEQ ID NO: 16);

(l) a twelfth peptide comprising the amino acid sequence EQPEQPFPE (SEQ ID NO: 17) and the amino acid sequence EQPFPEQPQ (SEQ ID NO: 41);

(m) a thirteenth peptide comprising the amino acid sequence PFSEQEQPV (SEQ ID NO: 18);

(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 19);

(p) a sixteenth peptide comprising the amino acid sequence PFPQPELPY (SEQ ID NO: 1) and the amino acid sequence PQPELPYPY (SEQ ID NO: 43);

(q) a seventeenth peptide comprising the amino acid sequence EQPFPEQPI (SEQ ID NO: 20) and the amino acid sequence PFPEQPIPE (SEQ ID NO: 21); and

(r) an eighteenth peptide comprising the amino acid sequence PQPYPEQPQ (SEQ ID NO: 23) and the amino acid sequence PYPEQPQPF (SEQ ID NO: 22).

In some embodiments,

(a) the first peptide comprises the amino acid sequence LQPFPQPELPYPQPQ (SEQ ID NO: 45);

(b) the second peptide comprises the amino acid sequence QPFPQPEQPFPWQP (SEQ ID NO: 46);

(c) the third peptide comprises the amino acid sequence PEQPIPEQPQPYPQQ (SEQ ID NO: 47);

(d) the fourth peptide comprises the amino acid sequence QPFPQPEQPIPVQPEQS (SEQ ID NO: 48);

(e) the fifth peptide comprises the amino acid sequence QPFPQPEQPTPIQPEQP (SEQ ID NO: 49);

(f) the sixth peptide comprises the amino acid sequence QPFPQPEQPFPLQPEQP (SEQ ID NO: 50);

(g) the seventh peptide comprises the amino acid sequence QPFPQPEQPFSQQ (SEQ ID NO: 51);

(h) the eighth peptide comprises the amino acid sequence PQPYPEQPQPFPQQ (SEQ ID NO: 52);

(i) the ninth peptide comprises the amino acid sequence QPFPEQPEQIIPQQP (SEQ ID NO: 53);

(j) the tenth peptide comprises the amino acid sequence SGEGSFQPSQENPQ (SEQ ID NO: 54);

(k) the eleventh peptide comprises the amino acid sequence GQQGYYPTSPQQSG (SEQ ID NO: 55);

(l) the twelfth peptide comprises the amino acid sequence PEQPEQPFPEQPQQ (SEQ ID NO: 56);

(m) the thirteenth peptide comprises the amino acid sequence QPPFSEQEQPVLPQ (SEQ ID NO: 57);

(n) the fourteenth peptide comprises the amino acid sequence PEQPFPEQPIPEQPQPYP (SEQ ID NO: 44);

(o) the fifteenth peptide comprises the amino acid sequence QPYPQPELPYPQPQ (SEQ ID NO: 58);

(p) the sixteenth peptide comprises the amino acid sequence QPFPQPELPYPYPQ (SEQ ID NO: 59);

(q) the seventeenth peptide comprises the amino acid sequence PQEQPFPEQPIPEQP (SEQ ID NO: 60); and

(r) the eighteenth peptide comprises the amino acid sequence QPQPYPEQPQPFPQQ (SEQ ID NO: 61).

In some embodiments of any one of the methods provided herein, the composition comprises at least one peptide, the at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23). In some embodiments of any one of the methods provided herein, the composition comprises at least one (e.g., two, three, four, five, six, seven, eight, nine, ten, eleven, twelve, thirteen, fourteen, fifteen, or sixteen) peptide comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23).

In some embodiments of any one of the methods provided herein, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9); (f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 15);

(k) a eleventh peptide comprising the amino acid sequence QGYYPTSPQ (SEQ ID NO: 16);

(l) a twelfth peptide comprising the amino acid sequence EQPEQPFPE (SEQ ID NO: 17);

(m) a thirteenth peptide comprising the amino acid sequence PFSEQEQPV (SEQ ID NO: 18);

(n) a fourteenth peptide comprising the amino acid sequence PYPQPELPY (SEQ ID NO: 19);

(o) a fifteenth peptide comprising the amino acid sequence EQPFPEQPI (SEQ ID NO: 20) and the amino acid sequence PFPEQPIPE (SEQ ID NO: 21); and

(p) a sixteenth peptide comprising the amino acid sequence PYPEQPQPF (SEQ ID NO: 22) and the amino acid sequence PQPYPEQPQ (SEQ ID NO: 23).

In some embodiments:

(a) the first peptide comprises the amino acid sequence PFPQPELPYPQP (SEQ ID NO: 24);

(b) the second peptide comprises the amino acid sequence PFPQPEQPFPWQ (SEQ ID NO: 25);

(c) the third peptide comprises the amino acid sequence EQPIPEQPQPYP (SEQ ID NO: 26);

(d) the fourth peptide comprises the amino acid sequence PFPQPEQPIPVQ (SEQ ID NO: 27);

(e) the fifth peptide comprises the amino acid sequence PEQPIPVQPEQS (SEQ ID NO: 28);

(f) the sixth peptide comprises the amino acid sequence PFPQPEQPTPIQ (SEQ ID NO: 29);

(g) the seventh peptide comprises the amino acid sequence PEQPTPIQPEQP (SEQ ID NO: 30);

(h) the eighth peptide comprises the amino acid sequence PFPQPEQPFPLQ (SEQ ID NO: 31);

(i) the ninth peptide comprises the amino acid sequence PEQPFPLQPEQP (SEQ ID NO: 32);

(j) the tenth peptide comprises the amino acid sequence GEGSFQPSQENP (SEQ ID NO: 33);

(k) the eleventh peptide comprises the amino acid sequence QQGYYPTSPQQS (SEQ ID NO: 34);

(l) the twelfth peptide comprises the amino acid sequence PEQPEQPFPEQP (SEQ ID NO: 35);

(m) the thirteenth peptide comprises the amino acid sequence PPFSEQEQPVLP (SEQ ID NO: 36);

(n) the fourteenth peptide comprises the amino acid sequence PYPQPELPYPQP (SEQ ID NO: 37);

(o) the fifteenth peptide comprises the amino acid sequence EQPFPEQPIPEQ (SEQ ID NO: 38); and

(p) the sixteenth peptide comprises the amino acid sequence PQPYPEQPQPFP (SEQ ID NO: 39).

In some embodiments of any one of the methods provided herein, the composition comprises at least four (e.g., five, six, seven, eight, nine, ten, eleven, twelve, thirteen, fourteen, fifteen or sixteen) of the peptides. In some embodiments of any one of the methods provided herein, the composition comprises (or consists of) the peptides in (a)-(p). In some embodiments of any one of the methods provided herein, at least one of the peptides comprises an N-terminal pyroglutamate and/or a C-terminal amide group. In some embodiments of any one of the methods provided herein, each of the peptides comprises an N-terminal pyroglutamate and/or a C-terminal amide group.

In some embodiments of any one of the methods provided herein, the composition comprises at least one of:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 63) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 40);

(viii) an eighth peptide comprising the amino acid sequence PYPEQPQPF (SEQ ID NO: 22);

(ix) a ninth peptide comprising the amino acid sequence PFPEQPEQIIP (SEQ ID NO: 64);

(x) a tenth peptide comprising the amino acid sequence EGSFQPSQE (SEQ ID NO: 15);

(xi) an eleventh peptide comprising the amino acid sequence QGYYPTSPQ (SEQ ID NO: 16);

(xii) a twelfth peptide comprising the amino acid sequence EQPEQPFPEQPQ (SEQ ID NO: 65);

(xiii) a thirteenth peptide comprising the amino acid sequence PFSEQEQPV (SEQ ID NO: 18);

(xiv) a fourteenth peptide comprising the amino acid sequence EQPFPEQPI (SEQ ID NO: 20) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 19);

(xvi) a sixteenth peptide comprising the amino acid sequence PFPQPELPY (SEQ ID NO: 1) and the amino acid sequence PQPELPYPY (SEQ ID NO: 43); and

(xvii) a seventeenth peptide comprising the amino acid sequence EQPFPEQPI (SEQ ID NO: 20).

In some embodiments of any one of the methods provided herein,

(i) the first peptide comprises the amino acid sequence LQPFPQPELPYPQPQ (SEQ ID NO: 45);

(ii) the second peptide comprises the amino acid sequence QPFPQPEQPFPWQP (SEQ ID NO: 46);

(iii) the third peptide comprises the amino acid sequence PEQPIPEQPQPYPQQ (SEQ ID NO: 47);

(iv) the fourth peptide comprises the amino acid sequence QPFPQPEQPIPVQPEQS (SEQ ID NO: 48);

(v) the fifth peptide comprises the amino acid sequence QPFPQPEQPTPIQPEQP (SEQ ID NO: 49);

(vi) the sixth peptide comprises the amino acid sequence QPFPQPEQPFPLQPEQP (SEQ ID NO: 50);

(vii) the seventh peptide comprises the amino acid sequence QPFPQPEQPFSQQ (SEQ ID NO: 51);

(viii) the eighth peptide comprises the amino acid sequence PQPYPEQPQPFPQQ (SEQ ID NO: 52);

(ix) the ninth peptide comprises the amino acid sequence QPFPEQPEQIIPQQP (SEQ ID NO: 53);

(x) the tenth peptide comprises the amino acid sequence SGEGSFQPSQENPQ (SEQ ID NO: 54);

(xi) the eleventh peptide comprises the amino acid sequence GQQGYYPTSPQQSG (SEQ ID NO: 55);

(xii) the twelfth peptide comprises the amino acid sequence PEQPEQPFPEQPQQ (SEQ ID NO: 56);

(xiii) the thirteenth peptide comprises the amino acid sequence QPPFSEQEQPVLPQ (SEQ ID NO: 57);

(xiv) the fourteenth peptide comprises the amino acid sequence PEQPFPEQPIPEQPQPYP (SEQ ID NO: 44);

(xv) the fifteenth peptide comprises the amino acid sequence QPYPQPELPYPQPQ (SEQ ID NO: 58);

(xvi) the sixteenth peptide comprises the amino acid sequence QPFPQPELPYPYPQ (SEQ ID NO: 59); and

(xvii) the seventeenth peptide comprises the amino acid sequence EQPFPEQPI (SEQ ID NO: 20).

“First”, “second”, “third”, etc. are not meant to imply an order of use or importance, unless specifically stated otherwise. In some embodiments of any one of the compositions provided, the peptides are each individually 8-50 amino acids in length. In some embodiments, the composition comprises at least one, at least two, at least three, at least four, at least five, at least six, at least seven, at least eight, at least nine, at least ten, at least eleven, at least twelve, at least thirteen, at least fourteen, at least fifteen or more of the peptides. In some embodiments, the composition comprises the first, second, and third peptides. In some embodiments, the composition comprises the first, second, third, fourth, fifth, sixth, seventh, eighth, ninth, tenth, eleventh, twelfth, and thirteenth peptides. In some embodiments, the composition comprises the second, fourth, fifth, sixth, seventh, eighth, ninth, tenth, eleventh, twelfth, thirteenth, fourteenth, fifteenth, and sixteenth peptides. In some embodiments, the composition comprises the first, second, third, fourth, fifth, sixth, eighth, ninth, tenth, eleventh, twelfth, and thirteenth peptides. In some embodiments, the composition comprises the second, fourth, fifth, sixth, eighth, ninth, tenth, eleventh, twelfth, thirteenth, fourteenth, fifteenth, and sixteenth peptides. In some embodiments, the composition comprises the second, third, fourth, fifth, sixth, seventh, eighth, ninth, tenth, eleventh, twelfth, thirteenth, fifteenth, sixteenth, and seventeenth peptides. In some embodiments, the composition comprises the second, third, fourth, fifth, sixth, eighth, ninth, tenth, eleventh, twelfth, thirteenth, fifteenth, sixteenth, and seventeenth peptides. In some embodiments, the composition comprises the first, second, third, fourth, fifth, sixth, tenth, eleventh, twelfth, thirteenth, fifteenth, seventeenth, and eighteenth peptides. In some embodiments, at least one of the peptides comprises an N-terminal pyroglutamate and/or a C-terminal amide group. In some embodiments, each of the peptides comprises an N-terminal pyroglutamate and/or a C-terminal amide group. In some embodiments, each of the peptides are present in an amount of 2.5 ug/mL in the composition. In some embodiments, each of the peptides are present in an amount of 5 ug/mL in the composition. In some embodiments, each of the peptides are present in an amount of 10 ug/mL in the composition. In some embodiments, each of the peptides are present in an amount of 20 ug/mL in the composition. In some embodiments, each of the peptides are present in an amount of 25 ug/mL in the composition. In some embodiments, each of the peptides are present in an amount of 50 ug/mL in the composition. In some embodiments, each of the peptides are present in an amount of 5 uM in the composition. In some embodiments, each of the peptides are present in an amount of 10 uM in the composition. In some embodiments, each of the peptides are present in an amount of 25 uM in the composition. In some embodiments, each of the peptides are present in an amount of 50 uM in the composition. Any one of the aforementioned compositions or peptide combinations may be used in any one of the methods provided herein.

Modifications to a gluten peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A gluten peptide may also be chemically modified at the level of amino acid side chains, of amino acid chirality, and/or of the peptide backbone.

Particular changes can be made to a gluten peptide to improve resistance to degradation or optimize solubility properties or otherwise improve bioavailability compared to the parent gluten peptide, thereby providing gluten peptides having similar or improved therapeutic, diagnostic and/or pharmacokinetic properties. A preferred such modification includes the use of an N-terminal acetyl group or pyroglutamate and/or a C-terminal amide. Such modifications have been shown in the art to significantly increase the half-life and bioavailability of the peptides compared to the parent peptides having a free N- and C-terminus (see, e.g., PCT Publication No.: WO/2010/060155). In some embodiments, a gluten peptide comprises an N-terminal acetyl group or pyroglutamate group, and/or a C-terminal amide group. In some embodiments of any one of the compositions or methods provided herein, the first, second and/or third peptides described above comprise an N-terminal acetyl group or pyroglutamate group, and/or a C-terminal amide group. In some embodiments of any one of the compositions or methods provided herein, the first, second, third, fourth, fifth, sixth, seventh, eighth, ninth, tenth, eleventh, twelfth, and/or thirteenth peptides described above comprise an N-terminal acetyl group or pyroglutamate group, and/or a C-terminal amide group. In some embodiments of any one of the compositions or methods provided herein, the second, fourth, fifth, sixth, seventh, eighth, ninth, tenth, eleventh, twelfth, thirteenth, fourteenth, fifteenth, and/or sixteenth peptides described above comprise an N-terminal acetyl group or pyroglutamate group, and/or a C-terminal amide group. In some embodiments of any one of the compositions or methods provided herein, the first, second, third, fourth, fifth, sixth, eighth, ninth, tenth, eleventh, twelfth, and/or thirteenth peptides described above comprise an N-terminal acetyl group or pyroglutamate group, and/or a C-terminal amide group. In some embodiments of any one of the compositions or methods provided herein, the second, fourth, fifth, sixth, eighth, ninth, tenth, eleventh, twelfth, thirteenth, fourteenth, fifteenth, and/or sixteenth peptides described above comprise an N-terminal acetyl group or pyroglutamate group, and/or a C-terminal amide group. In some embodiments, the first, second, third, fourth, fifth, sixth, tenth, eleventh, twelfth, thirteenth, fifteenth, seventeenth, and eighteenth peptides described above comprise an N-terminal acetyl group or pyroglutamate group, and/or a C-terminal amide group.

### Peptide Production

The peptides described herein (e.g., gluten peptides) can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides.

For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of 5V40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or 5V40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent. In some embodiments, the pharmaceutically acceptable salt is a trifluoroacetate (TFA) salt or an acetate salt

### Gluten Challenge

In some embodiments, any one of the methods provided herein comprise a gluten challenge or a sample obtained from a subject before, during, or after a gluten challenge. Generally, a gluten challenge comprises administering to the subject a composition comprising wheat, rye, or barley, or one or more peptides thereof (e.g., a composition comprising a wheat gliadin, a rye secalin, or a barley hordein, or one or more peptides thereof), in some form for a defined period of time in order to activate the immune system of the subject, e.g., through activation of wheat-, rye- and/or barley-reactive T cells and/or mobilization of such T cells in the subject. Methods of gluten challenges are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L, Guandalini S; Gastroenterology. 2009; 137(6):1912-33. Celiac disease: from pathogenesis to novel therapies. Schuppan D, Junker Y, Barisani D; J Dent Res. 2008; 87(12):1100-1107. Orally based diagnosis of celiac disease: current perspectives. Pastore L, Campisi G, Compilato D, and Lo Muzio L; Gastroenterology. 2001; 120:636-651. Current Approaches to Diagnosis and Treatment of Celiac Disease: An Evolving Spectrum. Fasano A and Catassi C; Clin Exp Immunol. 2000; 120:38-45. Local challenge of oral mucosa with gliadin in patients with coeliac disease. Lahteenoja M, Maki M, Viander M, Toivanen A, Syrjanen S; Clin Exp Immunol. 2000; 120:10-11. The mouth-an accessible region for gluten challenge. Ellis H and Ciclitira P; Clinical Science. 2001; 101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005; 54:1217-1223. T cells in peripheral blood after gluten challenge in coeliac disease. Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). As described herein, it has been found that IP-10 levels were elevated in subjects with Celiac disease, even in the absence of a gluten challenge. Accordingly, in some embodiments, any one of the methods provided is performed on a sample from a subject who has not undergone a gluten challenge (e.g., been administered gluten for at least 3 days after a period of at least 1 week, 1 month, 1 year or more of being on a gluten-free diet) within 1 week, 2 weeks, 3 weeks, 4 weeks, or more of the sample being obtained from the subject. In other embodiments, any one of the methods provided herein comprises performing a gluten challenge on the subject or obtaining a sample from a subject before, during or after a gluten challenge, where the gluten challenge is for less than 3 days.

In some embodiments, the challenge comprises administering a composition comprising wheat, barley and/or rye, or one or more peptides thereof. In some embodiments, the wheat is wheat flour, the barely is barley flour, and the rye is rye flour. In some embodiments, the challenge comprises administering a composition comprising a wheat gliadin, a barley hordein and/or a rye secalin, or one or more peptides thereof, to the subject prior to determining a T cell response as described herein.

In some embodiments, the composition is administered to the subject more than once prior to determining the level of IP-10, and a sample is obtained from the subject after administration of the composition. In some embodiments, administration is daily for 1 or 2 days. In some embodiments, administration is daily for 1, 2, or 3 days. In some embodiments, administration is more than once a day (e.g., twice a day) for 1 or 2 days. In some embodiments, administration is more than once a day (e.g., twice a day) for 1, 2, or 3 days. In some embodiments, the sample is obtained from the subject within 24 hours of administration of the composition. In some embodiments, the sample is obtained from the subject within 1, 2, 3, 4 or 5 days after administration of the composition. In some embodiments, the subject has been on a gluten-free diet for at least 4 weeks prior to commencing the gluten challenge.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a sample is obtained from a subject before, during, and/or after a gluten challenge as described herein.

### Other Testing

In some embodiments of any one of the methods provided, methods described herein further comprise other testing of a subject (e.g., based on the results of the methods described herein). As used herein, “other testing” describes use of at least one additional diagnostic method in addition to the methods provided herein. Any diagnostic method or combinations thereof for Celiac disease is contemplated as other testing. Exemplary other testing includes, but is not limited to, intestinal biopsy, serology (measuring the levels of one or more antibodies present in the serum), genotyping (see, e.g., Walker-Smith J A, et al. Arch Dis Child 1990), and measurement of a T cell response. Such other testing may be performed as part of the methods described herein or after the methods described herein (e.g., as a companion diagnostic), or before use of the methods described herein (e.g., as a first-pass screen to eliminate certain subjects before use of the methods described herein, e.g., eliminating those that do not have one or more HLA-DQA and HLA-DQB susceptibility alleles). In some embodiments of any one of the methods provided, no other testing is required to assess the subject's Celiac disease status, for example, having or not having Celiac disease.

Detection of serum antibodies (serology) is contemplated. The presence of such serum antibodies can be detected using methods known to those of skill in the art, e.g., by ELISA, histology, cytology, immunofluorescence or western blotting. Such antibodies include, but are not limited to: IgA anti-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

T cell response tests are also contemplated as other testing. In some embodiments, a T cell response test comprises contacting a sample comprising a T cell with at least one gluten peptide and measuring a T cell response in the sample. In some embodiments, a T cell response is measured by measuring a level of IFN-γ, where an increased level of IFN-γ compared to a control level (e.g., a level of IFN-γ in a sample that has not been contacted with a gluten peptide) may identify a subject as having Celiac disease. T cell response tests are known in the art (see, e.g., PCT Publication Nos.: WO/2001/025793, WO/2003/104273, WO/2005/105129, and WO/2010/060155).

### Treatment

In some embodiments of any one of the methods provided herein, the methods described herein further comprise a treatment step, such as treating a subject identified as having or likely as having Celiac disease. In some embodiments of any one of the methods provided, the methods comprise a step where information regarding treatment is provided to the subject. Such information can be given orally or in written form, such as with written materials. Written materials may be in an electronic form. Any known treatment of Celiac disease is contemplated herein. Exemplary treatments include, e.g., a gluten-free diet. Other exemplary treatments include endopeptidases, such as ALV003 (Alvine) and AT1001 (Alba), agents that inhibit transglutaminase activity, agents that block peptide presentation by HLA DQ2.5, or oral resins that bind to gluten peptides and reduce their bioavailability.

Compositions comprising gluten peptides for use in treating Celiac disease are known in the art (see, e.g., PCT Publication Nos.: WO/2001/025793, WO/2003/104273, WO/2005/105129, and WO/2010/060155, which are incorporated herein by reference in their entirety, including the gluten peptides in particular). In some embodiments, the composition comprises at least one of: (i) a first peptide comprising the amino acid sequence PFPQPELPY (SEQ ID NO: 1) and PQPELPYPQ (SEQ ID NO: 2), (ii) a second peptide comprising the amino acid sequence PFPQPEQPF (SEQ ID NO: 3) and PQPEQPFPW (SEQ ID NO: 4), or (iii) a third peptide comprising the amino acid sequence PIPEQPQPY (SEQ ID NO: 6). In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 45); the second peptide comprises the amino acid sequence QPFPQPEQPFPWQP (SEQ ID NO: 46); and/or the third peptide comprises the amino acid sequence PEQPIPEQPQPYPQQ (SEQ ID NO: 47). Modifications to such peptides, e.g., an N-terminal pyro-glutamate and/or C-terminal amide, are contemplated and described herein. In some embodiments, the first peptide comprises the amino acid sequence ELQPFPQPELPYPQPQ (SEQ ID NO: 232), wherein the N-terminal glutamate is a pyroglutamate and the C-terminal glutamine is amidated; the second peptide comprises the amino acid sequence EQPFPQPEQPFPWQP (SEQ ID NO: 233), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 234), wherein the N-terminal glutamate is a pyroglutamate and the C-terminal glutamine is amidated. In some embodiments, the first peptide consists of the amino acid sequence ELQPFPQPELPYPQPQ (SEQ ID NO: 232), wherein the N-terminal glutamate is a pyroglutamate and the C-terminal glutamine is amidated; the second peptide consists of the amino acid sequence EQPFPQPEQPFPWQP (SEQ ID NO: 233), wherein the N-terminal glutamate is a pyroglutamate and the C-terminal proline is amidated (e.g., the free C-terminal COO is amidated); and/or the third peptide consists of the amino acid sequence EPEQPIPEQPQPYPQQ (SEQ ID NO: 234), wherein the N-terminal glutamate is a pyroglutamate and the C-terminal glutamine is amidated (e.g., the free C-terminal COO is amidated). In some embodiments, the composition comprises 150 micrograms of the peptides (i.e., 50 micrograms of the first peptide and an equimolar amount of each of the second and third peptides). In some embodiments, the composition comprises 300 micrograms of the peptides (i.e., 100 micrograms of the first peptide and an equimolar amount of each of the second and third peptides). Any one of these compositions may be for use in any one of the methods or kits provided herein.

In some embodiments of any one of the method or kits provided, a treatment comprises a composition comprising at least one (e.g., at least four) gluten peptides as described herein. In some embodiments, the composition is a composition described in the Examples provided. Modifications to such peptides, e.g., an N-terminal pyro-glutamate and/or C-terminal amide, are contemplated and described herein.

Treatments may be administered through any method known in the art. Pharmaceutical compositions suitable for each administration route are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005). In some embodiments, a treatment, e.g., a composition comprising a gluten peptide, such as those provided herein, is administered via injection, such as intradermal injection.

The peptides or other compositions provided herein may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. Compositions, such as pharmaceutical compositions, may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the pharmaceutical composition. The carrier can be any of those conventionally used and is limited only by chemico-physical considerations, such as solubility and lack of reactivity with the active agent, and by the route of administration. Suitable carriers for the pharmaceutical composition include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers. Other carriers are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005).

The pharmaceutical composition(s) may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection. Examples of appropriate delivery mechanisms for intradermal administration include, but are not limited to, syringes, needles, and osmotic pumps.

It can be advantageous to formulate the active agent in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Alternatively, the compositions may be presented in multi-dose form. Examples of dosage units include sealed ampoules and vials and may be stored in a freeze-dried condition requiring only the addition of the sterile liquid carrier immediately prior to use.

The actual amount administered (or dose or dosage) and the rate and time-course of administration will depend on the nature and severity of the condition being treated as well as the characteristics of the subject to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the subject, the site of delivery, the method of administration and other factors known to practitioners. Examples of techniques and protocols can be found in, e.g., Remington's Pharmaceutical Sciences, 16th Ed. Mack Publishing Company, 1980 and Remington: The Science and Practice of Pharmacy, 21st Ed. Lippincott Williams & Wilkins, 2005. Effective amounts may be measured from ng/kg body weight to g/kg body weight per minute, hour, day, week or month.

As used herein, the terms “treat”, “treating”, and “treatment” include abrogating, inhibiting, slowing, or reversing the progression of a disease or condition, or ameliorating or preventing a clinical symptom of the disease (for example, Celiac disease). Treatment may include induction of immune tolerance (for example, to gluten or peptides thereof), modification of the cytokine secretion profile of the subject and/or induction of suppressor T cell subpopulations to secrete cytokines. Thus, a subject treated according to the disclosure preferably, in some embodiments, is able to eat at least wheat, rye, and/or barley without a significant T cell response which would normally lead to symptoms of Celiac disease. In some embodiments, an effective amount of a treatment is administered. The term “effective amount” means the amount of a treatment sufficient to provide the desired therapeutic or physiological effect when administered under appropriate or sufficient conditions.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Kits

Also disclosed herein are kits for measuring an immune response, e.g., by detecting IP-10 in a sample comprising a lymphocyte. In some embodiments, the kit comprises: (a) any one of the compositions comprising at least one gluten peptide as described herein and (b) a binding partner for IP-10. In some embodiments of any one of the kits provided herein, the kit further comprises an agent that recognizes the binding partner for IP-10. In some embodiments of any one of the kits provided herein, the kit further comprises a container for blood. In some embodiments of any one of the kits provided herein, the composition is contained within the container (e.g., dried onto the wall of the container).

In some embodiments of any one of the kits provided herein, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 40);

(h) an eighth peptide comprising the amino acid sequence PYPEQPQPF (SEQ ID NO: 22);

(i) a ninth peptide comprising the amino acid sequence PFPEQPEQI (SEQ ID NO: 42);

(j) a tenth peptide comprising the amino acid sequence EGSFQPSQE (SEQ ID NO: 15);

(k) an eleventh peptide comprising the amino acid sequence QGYYPTSPQ (SEQ ID NO: 16);

(l) a twelfth peptide comprising the amino acid sequence EQPEQPFPE (SEQ ID NO: 17) and the amino acid sequence EQPFPEQPQ (SEQ ID NO: 41);

(m) a thirteenth peptide comprising the amino acid sequence PFSEQEQPV (SEQ ID NO: 18);

(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 19);

(p) a sixteenth peptide comprising the amino acid sequence PFPQPELPY (SEQ ID NO: 1) and the amino acid sequence PQPELPYPY (SEQ ID NO: 43);

(q) a seventeenth peptide comprising the amino acid sequence EQPFPEQPI (SEQ ID NO: 20) and the amino acid sequence PFPEQPIPE (SEQ ID NO: 21); and

(r) an eighteenth peptide comprising the amino acid sequence PQPYPEQPQ (SEQ ID NO: 23) and the amino acid sequence PYPEQPQPF (SEQ ID NO: 22).

In some embodiments,

(a) the first peptide comprises the amino acid sequence LQPFPQPELPYPQPQ (SEQ ID NO: 45);

(b) the second peptide comprises the amino acid sequence QPFPQPEQPFPWQP (SEQ ID NO: 46);

(c) the third peptide comprises the amino acid sequence PEQPIPEQPQPYPQQ (SEQ ID NO: 47);

(d) the fourth peptide comprises the amino acid sequence QPFPQPEQPIPVQPEQS (SEQ ID NO: 48);

(e) the fifth peptide comprises the amino acid sequence QPFPQPEQPTPIQPEQP (SEQ ID NO: 49);

(f) the sixth peptide comprises the amino acid sequence QPFPQPEQPFPLQPEQP (SEQ ID NO: 50);

(g) the seventh peptide comprises the amino acid sequence QPFPQPEQPFSQQ (SEQ ID NO: 51);

(h) the eighth peptide comprises the amino acid sequence PQPYPEQPQPFPQQ (SEQ ID NO: 52);

(i) the ninth peptide comprises the amino acid sequence QPFPEQPEQIIPQQP (SEQ ID NO: 53);

(j) the tenth peptide comprises the amino acid sequence SGEGSFQPSQENPQ (SEQ ID NO: 54);

(k) the eleventh peptide comprises the amino acid sequence GQQGYYPTSPQQSG (SEQ ID NO: 55);

(l) the twelfth peptide comprises the amino acid sequence PEQPEQPFPEQPQQ (SEQ ID NO: 56);

(m) the thirteenth peptide comprises the amino acid sequence QPPFSEQEQPVLPQ (SEQ ID NO: 57);

(n) the fourteenth peptide comprises the amino acid sequence PEQPFPEQPIPEQPQPYP (SEQ ID NO: 44);

(o) the fifteenth peptide comprises the amino acid sequence QPYPQPELPYPQPQ (SEQ ID NO: 58);

(p) the sixteenth peptide comprises the amino acid sequence QPFPQPELPYPYPQ (SEQ ID NO: 59);

(q) the seventeenth peptide comprises the amino acid sequence PQEQPFPEQPIPEQP (SEQ ID NO: 60); and

(r) the eighteenth peptide comprises the amino acid sequence QPQPYPEQPQPFPQQ (SEQ ID NO: 61).

In some embodiments of any one of the kits provided herein, the composition comprises at least one of:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 63) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 40);

(viii) an eighth peptide comprising the amino acid sequence PYPEQPQPF (SEQ ID NO: 22);

(ix) a ninth peptide comprising the amino acid sequence PFPEQPEQIIP (SEQ ID NO: 64);

(x) a tenth peptide comprising the amino acid sequence EGSFQPSQE (SEQ ID NO: 15);

(xi) an eleventh peptide comprising the amino acid sequence QGYYPTSPQ (SEQ ID NO: 16);

(xii) a twelfth peptide comprising the amino acid sequence EQPEQPFPEQPQ (SEQ ID NO: 65);

(xiii) a thirteenth peptide comprising the amino acid sequence PFSEQEQPV (SEQ ID NO: 18);

(xiv) a fourteenth peptide comprising the amino acid sequence EQPFPEQPI (SEQ ID NO: 20) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 19);

(xvi) a sixteenth peptide comprising the amino acid sequence PFPQPELPY (SEQ ID NO: 1) and the amino acid sequence PQPELPYPY (SEQ ID NO: 43); and

(xvii) a seventeenth peptide comprising the amino acid sequence EQPFPEQPI (SEQ ID NO: 20).

In some embodiments,

(i) the first peptide comprises the amino acid sequence LQPFPQPELPYPQPQ (SEQ ID NO: 45);

(ii) the second peptide comprises the amino acid sequence QPFPQPEQPFPWQP (SEQ ID NO: 46);

(iii) the third peptide comprises the amino acid sequence PEQPIPEQPQPYPQQ (SEQ ID NO: 47);

(iv) the fourth peptide comprises the amino acid sequence QPFPQPEQPIPVQPEQS (SEQ ID NO: 48);

(v) the fifth peptide comprises the amino acid sequence QPFPQPEQPTPIQPEQP (SEQ ID NO: 49);

(vi) the sixth peptide comprises the amino acid sequence QPFPQPEQPFPLQPEQP (SEQ ID NO: 50);

(vii) the seventh peptide comprises the amino acid sequence QPFPQPEQPFSQQ (SEQ ID NO: 51);

(viii) the eighth peptide comprises the amino acid sequence PQPYPEQPQPFPQQ (SEQ ID NO: 52);

(ix) the ninth peptide comprises the amino acid sequence QPFPEQPEQIIPQQP (SEQ ID NO: 53);

(x) the tenth peptide comprises the amino acid sequence SGEGSFQPSQENPQ (SEQ ID NO: 54);

(xi) the eleventh peptide comprises the amino acid sequence GQQGYYPTSPQQSG (SEQ ID NO: 55);

(xii) the twelfth peptide comprises the amino acid sequence PEQPEQPFPEQPQQ (SEQ ID NO: 56);

(xiii) the thirteenth peptide comprises the amino acid sequence QPPFSEQEQPVLPQ (SEQ ID NO: 57);

(xiv) the fourteenth peptide comprises the amino acid sequence PEQPFPEQPIPEQPQPYP (SEQ ID NO: 44);

(xv) the fifteenth peptide comprises the amino acid sequence QPYPQPELPYPQPQ (SEQ ID NO: 58);

(xvi) the sixteenth peptide comprises the amino acid sequence QPFPQPELPYPYPQ (SEQ ID NO: 59); and

(xvii) the seventeenth peptide comprises the amino acid sequence EQPFPEQPI (SEQ ID NO: 20). In some embodiments, the composition comprises the first, second, and third peptides. In some embodiments, the composition comprises the first, second, third, fourth, fifth, sixth, seventh, eighth, ninth, tenth, eleventh, twelfth, and thirteenth peptides. In some embodiments, the composition comprises the second, fourth, fifth, sixth, seventh, eighth, ninth, tenth, eleventh, twelfth, thirteenth, fourteenth, fifteenth, and sixteenth peptides. In some embodiments, the composition comprises the first, second, third, fourth, fifth, sixth, eighth, ninth, tenth, eleventh, twelfth, and thirteenth peptides. In some embodiments, the composition comprises the second, fourth, fifth, sixth, eighth, ninth, tenth, eleventh, twelfth, thirteenth, fourteenth, fifteenth, and sixteenth peptides. In some embodiments, the composition comprises the second, third, fourth, fifth, sixth, seventh, eighth, ninth, tenth, eleventh, twelfth, thirteenth, fifteenth, sixteenth, and seventeenth peptides. In some embodiments, the composition comprises the second, third, fourth, fifth, sixth, eighth, ninth, tenth, eleventh, twelfth, thirteenth, fifteenth, sixteenth, and seventeenth peptides. In some embodiments, the composition comprises the first, second, third, fourth, fifth, sixth, tenth, eleventh, twelfth, thirteenth, fifteenth, seventeenth, and eighteenth peptides. In some embodiments of any one of the kits provided herein, at least one of the peptides comprises an N-terminal pyroglutamate and/or a C-terminal amide group. In some embodiments of any one of the kits provided herein, each of the peptides comprises an N-terminal pyroglutamate and/or a C-terminal amide group.

In some embodiments of any one of the kits provided herein, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 15);

(k) a eleventh peptide comprising the amino acid sequence QGYYPTSPQ (SEQ ID NO: 16);

(l) a twelfth peptide comprising the amino acid sequence EQPEQPFPE (SEQ ID NO: 17);

(m) a thirteenth peptide comprising the amino acid sequence PFSEQEQPV (SEQ ID NO: 18);

(n) a fourteenth peptide comprising the amino acid sequence PYPQPELPY (SEQ ID NO: 19);

(o) a fifteenth peptide comprising the amino acid sequence EQPFPEQPI (SEQ ID NO: 20) and the amino acid sequence PFPEQPIPE (SEQ ID NO: 21); and

(p) a sixteenth peptide comprising the amino acid sequence PYPEQPQPF (SEQ ID NO: 22) and the amino acid sequence PQPYPEQPQ (SEQ ID NO: 23).

In some embodiments:

(a) the first peptide comprises the amino acid sequence PFPQPELPYPQP (SEQ ID NO: 24);

(b) the second peptide comprises the amino acid sequence PFPQPEQPFPWQ (SEQ ID NO: 25);

(c) the third peptide comprises the amino acid sequence EQPIPEQPQPYP (SEQ ID NO: 26);

(d) the fourth peptide comprises the amino acid sequence PFPQPEQPIPVQ (SEQ ID NO: 27);

(e) the fifth peptide comprises the amino acid sequence PEQPIPVQPEQS (SEQ ID NO: 28);

(f) the sixth peptide comprises the amino acid sequence PFPQPEQPTPIQ (SEQ ID NO: 29);

(g) the seventh peptide comprises the amino acid sequence PEQPTPIQPEQP (SEQ ID NO: 30);

(h) the eighth peptide comprises the amino acid sequence PFPQPEQPFPLQ (SEQ ID NO: 31);

(i) the ninth peptide comprises the amino acid sequence PEQPFPLQPEQP (SEQ ID NO: 32);

(j) the tenth peptide comprises the amino acid sequence GEGSFQPSQENP (SEQ ID NO: 33);

(k) the eleventh peptide comprises the amino acid sequence QQGYYPTSPQQS (SEQ ID NO: 34);

(l) the twelfth peptide comprises the amino acid sequence PEQPEQPFPEQP (SEQ ID NO: 35);

(m) the thirteenth peptide comprises the amino acid sequence PPFSEQEQPVLP (SEQ ID NO: 36);

(n) the fourteenth peptide comprises the amino acid sequence PYPQPELPYPQP (SEQ ID NO: 37);

(o) the fifteenth peptide comprises the amino acid sequence EQPFPEQPIPEQ (SEQ ID NO: 38); and

(p) the sixteenth peptide comprises the amino acid sequence PQPYPEQPQPFP (SEQ ID NO: 39).

In some embodiments of any one of the kits provided herein, the composition comprises at least four (e.g., five, six, seven, eight, nine, ten, eleven, twelve, thirteen, fourteen, fifteen or sixteen) of the peptides. In some embodiments of any one of the kits provided herein, the composition comprises (or consists of) the peptides in (a)-(p). In some embodiments of any one of the kits provided herein, at least one of the peptides comprises an N-terminal pyroglutamate and/or a C-terminal amide group. In some embodiments of any one of the kits provided herein, each of the peptides comprises an N-terminal pyroglutamate and/or a C-terminal amide group.

In some embodiments of any one of the kits provided herein, each of the peptides are present in an amount of 2.5 ug/mL in the composition. In some embodiments of any one of the kits provided herein, each of the peptides are present in an amount of 5 ug/mL in the composition. In some embodiments of any one of the kits provided herein, each of the peptides are present in an amount of 10 ug/mL in the composition. In some embodiments of any one of the kits provided herein, each of the peptides are present in an amount of 20 ug/mL in the composition. In some embodiments of any one of the kits provided herein, each of the peptides are present in an amount of 25 ug/mL in the composition. In some embodiments of any one of the kits provided herein, each of the peptides are present in an amount of 50 ug/mL in the composition. In some embodiments of any one of the kits provided herein, each of the peptides are present in an amount of 5 uM in the composition. In some embodiments of any one of the kits provided herein, each of the peptides are present in an amount of 10 uM in the composition. In some embodiments of any one of the kits provided herein, each of the peptides are present in an amount of 25 uM in the composition. In some embodiments of any one of the kits provided, each of the peptides are present in an amount of 50 uM in the composition.

In some embodiments of any one of the kits provided, the kit further comprises a second composition comprising the first, second and third peptides, wherein the second composition contains 10 micrograms of the first peptide and an equimolar amount of each of the second and third peptides; 15 micrograms of the first peptide and an equimolar amount of each of the second and third peptides; 20 micrograms of the first peptide and an equimolar amount of each of the second and third peptides; or 50 micrograms of the first peptide and an equimolar amount of each of the second and third peptides. In some embodiments, the second composition is formulated for intradermal injection (e.g., in 0.9% saline USP). The second composition may be housed within a container suitable for injection (e.g., a syringe).

Any suitable binding partner for IP-10 is contemplated. In some embodiments, the binding partner is any molecule that binds specifically to an IP-10 protein. As described herein, “binds specifically to an IP-10 protein” means that the molecule is more likely to bind to a portion of or the entirety of the IP-10 protein than to a portion of or the entirety of a non-IP-10 protein. In some embodiments of any one of the kits provided herein, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically to IP-10. Methods for producing peptide molecules and aptamers are well known in the art (see, e.g., published US Patent Application No. 2009/0075834, U.S. Pat. Nos. 7,435,542, 7,807,351, and 7,239,742). In some embodiments, the binding partner is any molecule that binds specifically to an IP-10 mRNA. As described herein, “binds specifically to an IP-10 mRNA” means that the molecule is more likely to bind to a portion of or the entirety of the IP-10 mRNA (e.g., by complementary base-pairing) than to a portion of or the entirety of a non-IP-10 mRNA or other non-IP-10 nucleic acid. In some embodiments of any one of the kits provided herein, the binding partner that binds specifically to IP-10 mRNA is a nucleic acid, e.g., a probe. Binding partners can be designed using the nucleotide and amino acid sequences of IP-10, which are associated with the IP-10 Genbank IDs provided herein. In some embodiments of any one of the kits provided herein, the binding partner for IP-10 is an anti-IP10 antibody or an antigen-binding fragment thereof.

In some embodiments of any one of the kits provided herein, any one of the kits provided comprises a first and second binding partner for IP-10. In some embodiments of any one of the kits provided herein, the first and second binding partners are antibodies or antigen binding fragments thereof. In some embodiments of any one of the kits provided herein, the second binding partner is bound to a surface. The second binding partner may be bound to the surface covalently or non-covalently. The second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments, the first binding partner for IP-10 is washed over the IP-10 bound to the second binding partner (e.g., as in a sandwich ELISA). The first binding partner may comprise a detectable label, or an agent that recognizes the first binding partner for IP-10 (e.g., a secondary antibody) may comprise a detectable label.

Any suitable agent that recognizes a binding partner for IP-10 is contemplated. In some embodiments, the binding partner is any molecule that binds specifically to the binding partner for IP-10. In some embodiments of any one of the kits provided herein, the agent is an antibody (e.g., a secondary antibody) or antigen-binding fragment thereof, such as Fab, F(ab)2, Fv, single chain antibodies, Fab and sFab fragments, F(ab′)2, Fd fragments, scFv, or dAb fragments. Agents also include other peptide molecules and aptamers that bind specifically to a binding partner for IP-10. In some embodiments of any one of the kits provided herein, the binding partner for IP-10 comprises a biotin moiety and the agent is a composition that binds to the biotin moiety (e.g., an avidin or streptavidin).

In some embodiments of any one of the kits provided, the binding partner for IP-10 and/or the agent comprise a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art are detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments of any one of the kits provided, the kit further comprises a negative control, e.g., a composition that does not comprise a gluten peptide, e.g., a saline solution or cell culture medium. In some embodiments of any one of the kits provided, the kit further comprises a positive control, e.g., a composition comprising IP-10 at a known concentration.

In some embodiments of any one of the kits provided, the kit comprises any combination of the components mentioned above.

In some embodiments of any one of the kits provided, the kit further comprises instructions for detecting IP-10 in a sample from a subject suspected of having Celiac disease. In some embodiments of any one of the kits provided, the instructions include any one of the methods as described herein. Instructions can be in any suitable form, e.g., as a printed insert or a label.

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Sambrook et al., Molecular Cloning: A Laboratory Manual, Cold Spring Harbour Laboratory Press (2012); T. A. Brown (editor), Essential Molecular Biology: A Practical Approach, Volumes 1 and 2, IRL Press (2000 and 2002); D. M. Glover and B. D. Hames (editors), Current Protocols in Molecular Biology, Greene Pub. Associates and Wiley-Interscience (1988, including all updates until present); Edward A. Greenfield (editor) Antibodies: A Laboratory Manual, Cold Spring Harbour Laboratory, (2013); and J. E. Coligan et al. (editors), Current Protocols in Immunology, John Wiley & Sons (including all updates until present).

In any one aspect or embodiment provided herein “comprising” may be replaced with “consisting essentially of” or “consisting of”.

### EXAMPLES

### Example 1

### Methods

HLA-DQ2.5-positive celiac disease subjects on gluten-free diet were used in this study. Blood was collected immediately before and 6 days after commencing 3-day oral gluten challenge. Whole blood or PBMCs were incubated with pools or single peptides derived from gluten or recall antigens. IFNγ and IP-10 levels were measured in plasma from the whole blood that was incubated in 96-well plates with peptides or peptide pools. Plasma cytokine/chemokine levels were measured by MAGPIX® multiplex bead assay (IFNγ and IP-10) or by ELISA (IFNγ and IP-10), and PBMC separated from the same blood sample were incubated in overnight IFNγ ELISpot assays.

The peptide pools used were:

Peptide pool 1-3 peptides (pE = pyroglutamate)

(SEQ ID NO: 235)

(pE)LQPFPQPELPYPQPQ-amide

(SEQ ID NO: 236)

(pE)QPFPQPEQPFPWQP-amide

(SEQ ID NO: 237)

(pE)PEQPIPEQPQPYPQQ-amide
Peptide pool 2-13 peptides (pE = pyroglutamate)

(SEQ ID NO: 238)

(pE)LQPFPQPELPYPQPQ-amide

(SEQ ID NO: 239)

(pE)QPFPQPEQPFPWQP-amide

(SEQ ID NO: 240)

(pE)PEQPIPEQPQPYPQQ-amide

(SEQ ID NO: 241)

(pE)QPFPQPEQPIPVQPEQS-amide

(SEQ ID NO: 242)

(pE)QPFPQPEQPTPIQPEQP-amide

(SEQ ID NO: 243)

(pE)QPFPQPEQPFPLQPEQP-amide

(SEQ ID NO: 244)

(pE)QPFPQPEQPFSQQ-amide

(SEQ ID NO: 245)

(pE)PQPYPEQPQPFPQQ-amide

(SEQ ID NO: 246)

(pE)QPFPEQPEQIIPQQP-amide

(SEQ ID NO: 247)

(pE)SGEGSFQPSQENPQ-amide

(SEQ ID NO: 248)

(pE)GQQGYYPTSPQQSG-amide

(SEQ ID NO: 249)

(pE)PEQPEQPFPEQPQQ-amide

(SEQ ID NO: 250)

(pE)QPPFSEQEQPVLPQ-amide
Peptide pool 3-14 peptides (pE = pyroglutamate)

(SEQ ID NO: 251)

(pE)QPFPQPEQPFPWQP-amide

(SEQ ID NO: 252)

(pE)QPFPQPEQPIPVQPEQS-amide

(SEQ ID NO: 253)

(pE)QPFPQPEQPTPIQPEQP-amide

(SEQ ID NO: 254)

(pE)QPFPQPEQPFPLQPEQP-amide

(SEQ ID NO: 255)

(pE)QPFPQPEQPFSQQ-amide

(SEQ ID NO: 256)

(pE)PQPYPEQPQPFPQQ-amide

(SEQ ID NO: 257)

(pE)QPFPEQPEQIIPQQP-amide

(SEQ ID NO: 258)

(pE)SGEGSFQPSQENPQ-amide

(SEQ ID NO: 259)

(pE)GQQGYYPTSPQQSG-amide

(SEQ ID NO: 260)

(pE)PEQPEQPFPEQPQQ-amide

(SEQ ID NO: 261)

(pE)QPPFSEQEQPVLPQ-amide

(SEQ ID NO: 262)

(pE)PEQPFPEQPIPEQPQPYP-amide

(SEQ ID NO: 263)

(pE)QPYPQPELPYPQPQ-amide

(SEQ ID NO: 264)

(pE)QPFPQPELPYPYPQ-amide

Each peptide in the above pools was designed to include at least one T cell epitope. The peptide pools were provided such that equimolar amounts of each peptide were present in each pool. A total gluten pool including 71 peptides capturing the majority of T cell epitopes in gluten was used as a control to simulate total gluten.

### Results

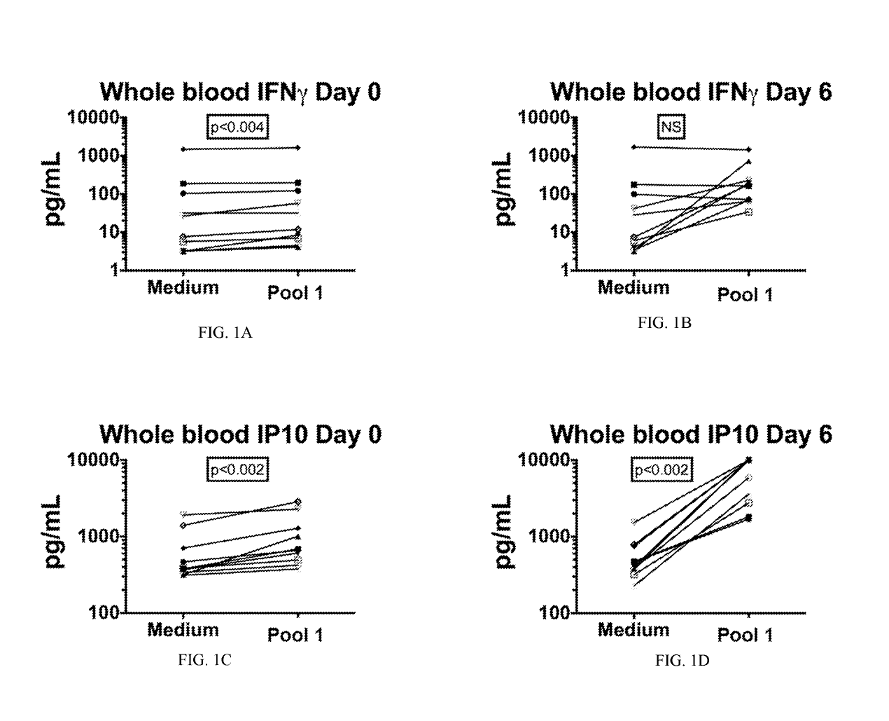

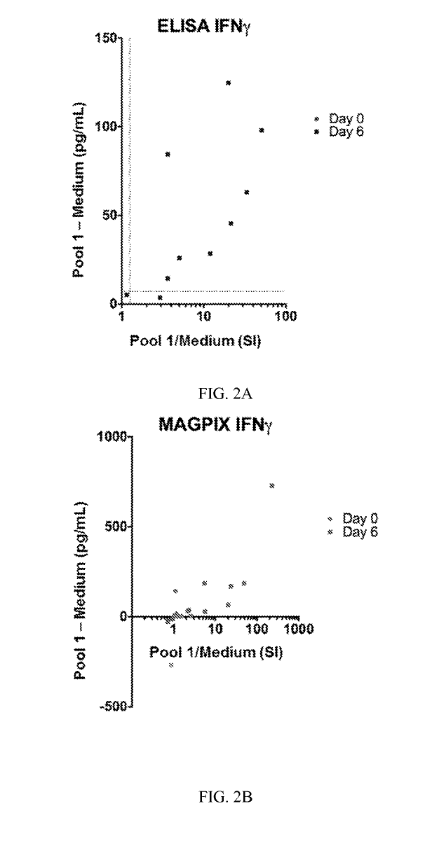

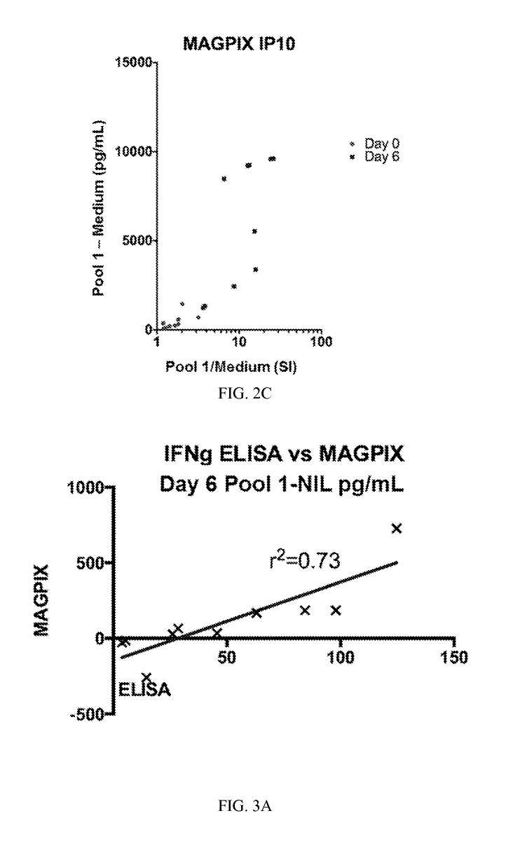

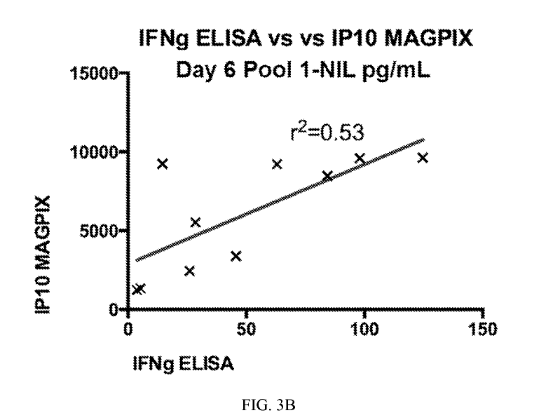

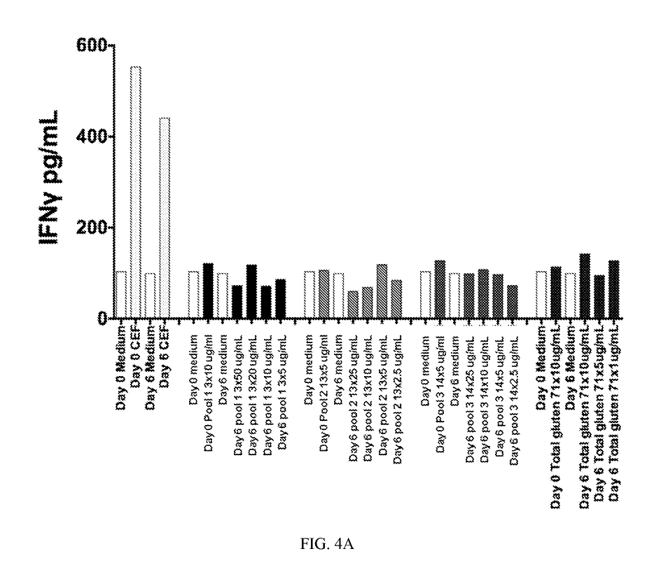

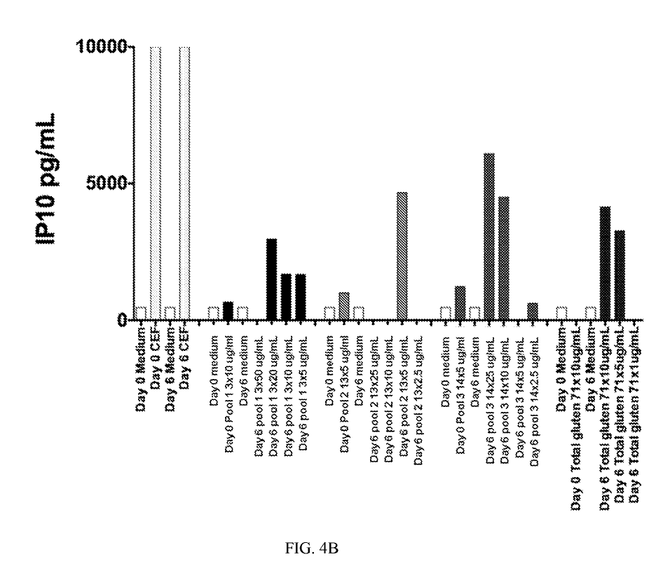

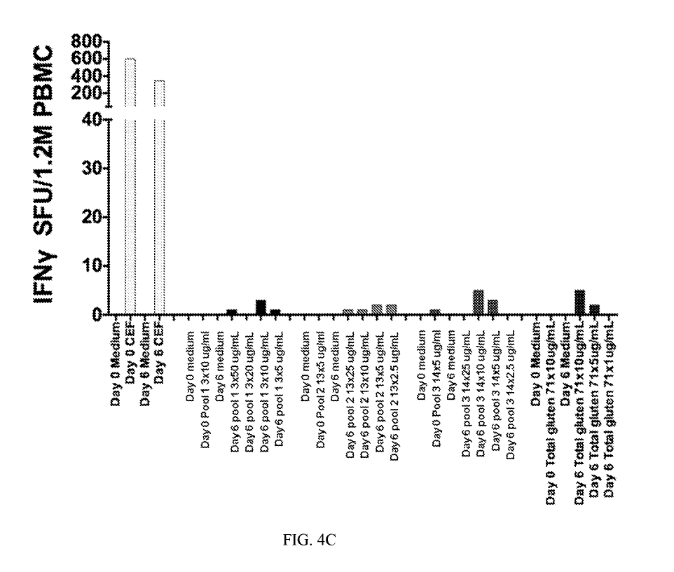

It was observed that IP-10 was elevated in plasma from blood incubated with peptide pool 1 6 days after commencing the oral gluten challenge. The elevation was more statistically signficant with IP-10 (p<0.002) than with IFN-γ (not significant) (FIGS. 1-3, Table 1 and 2). Using a cut-off of stimulation index >1.25 and cytokine level >100 pg/mL to indicate a positive result, the IP-10 assay using pool 1 was positive in 7/10 subjects prior to gluten challenge and 10/10 subjects after gluten challenge, whereas the IFN-γ assay using pool 1, with a cut-off of stimulation index >1.25 and cytokine level >7.2 pg/mL to indicate a positive result, was positive in 1/10 subjects pre-gluten challenge and 7/10 subjects after gluten challenge. Thus, the IP-10 assay was very effective both before and after gluten challenge. IP-10 levels were also elevated when blood was incubated with peptide pools 2 and 3 (FIG. 4).

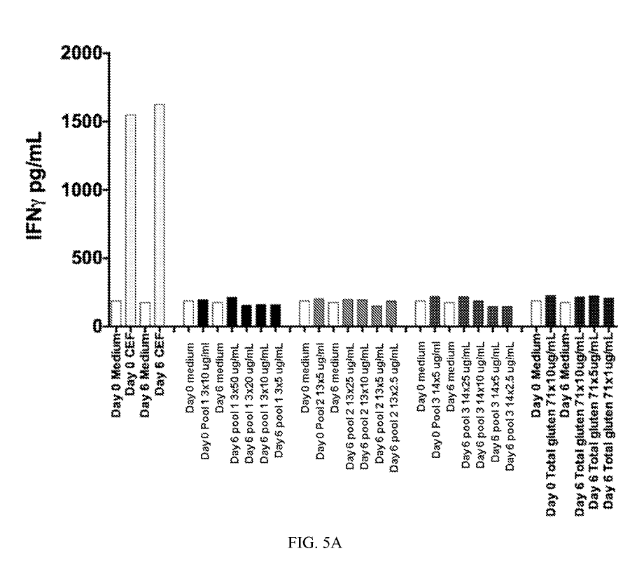

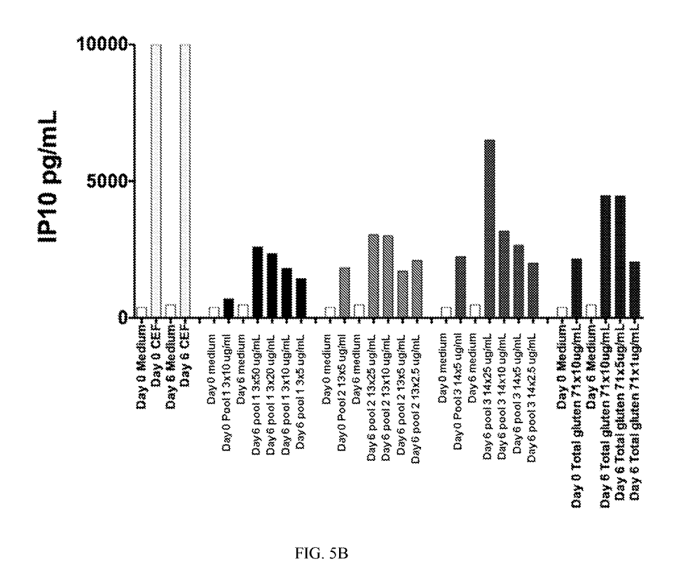

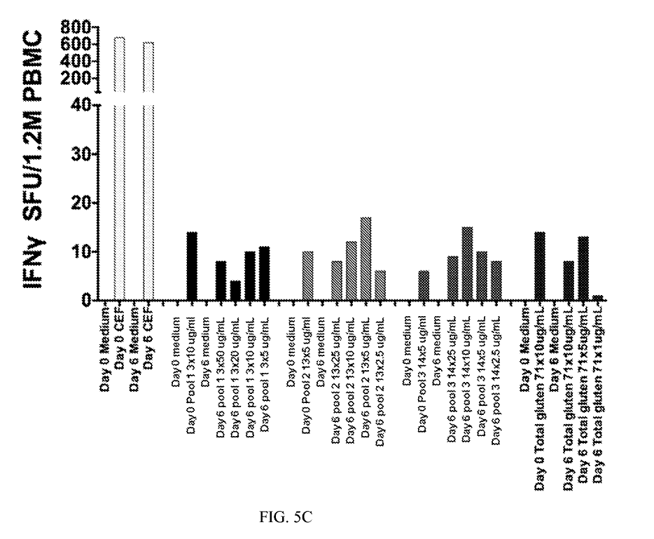

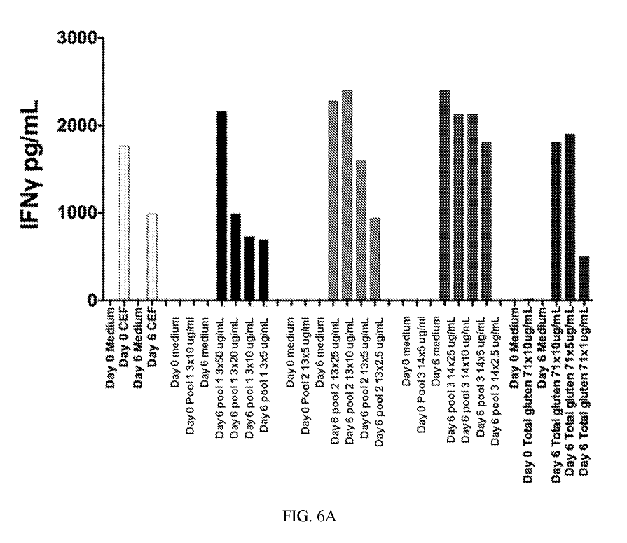

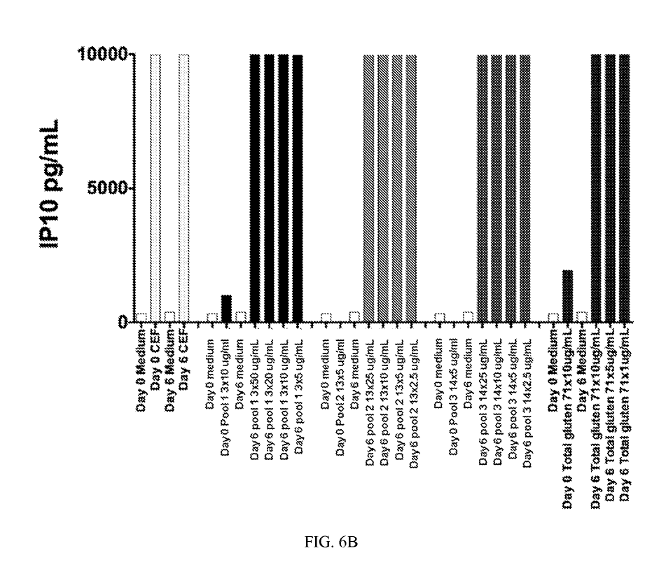

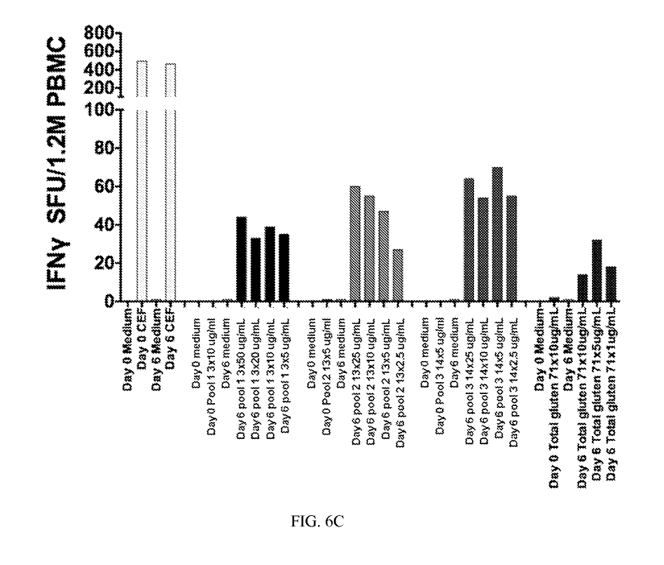

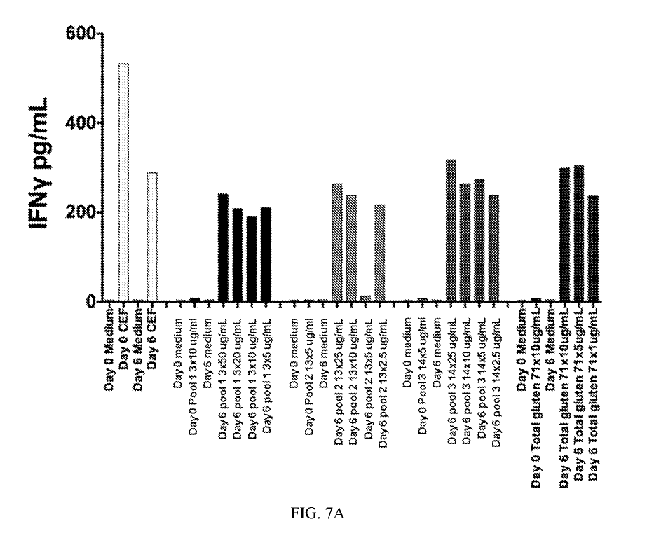

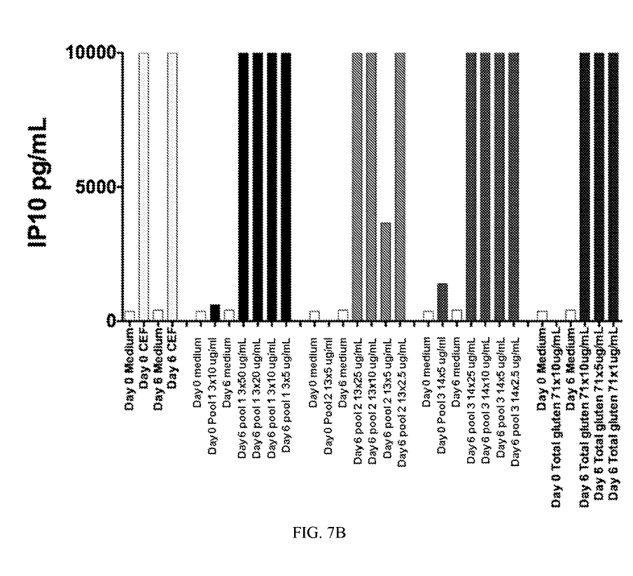

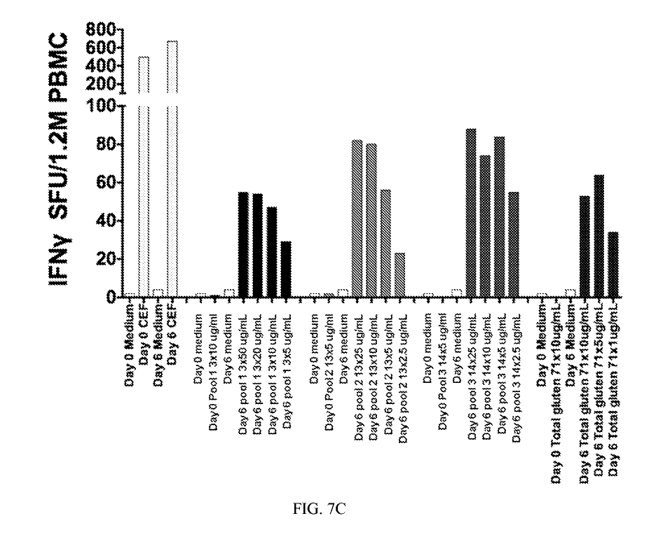

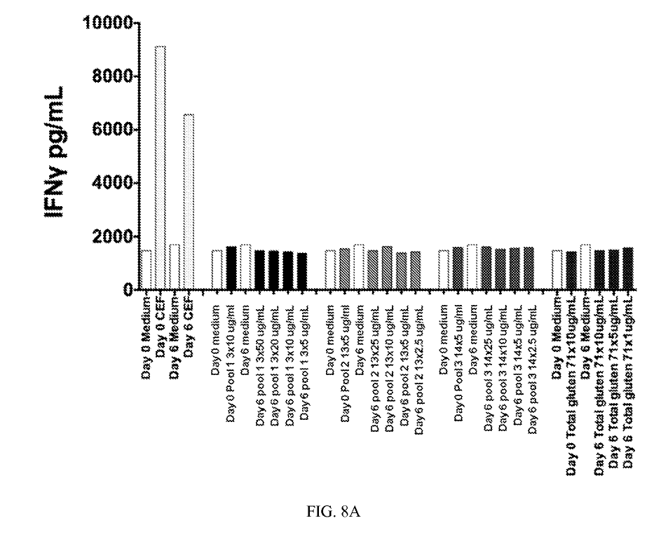

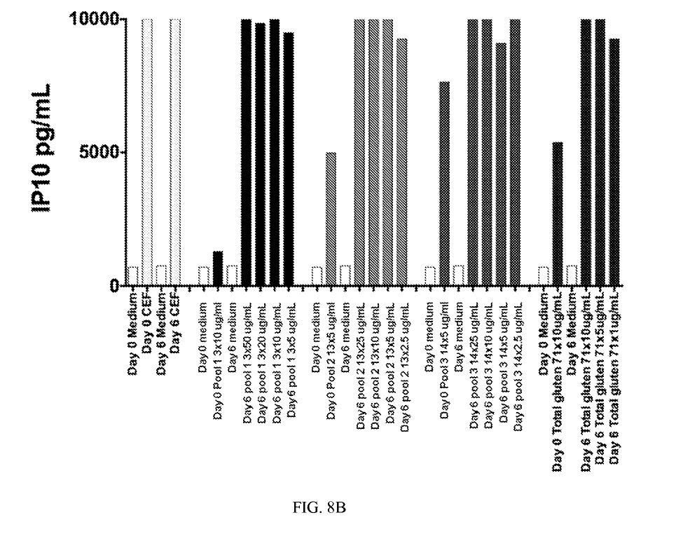

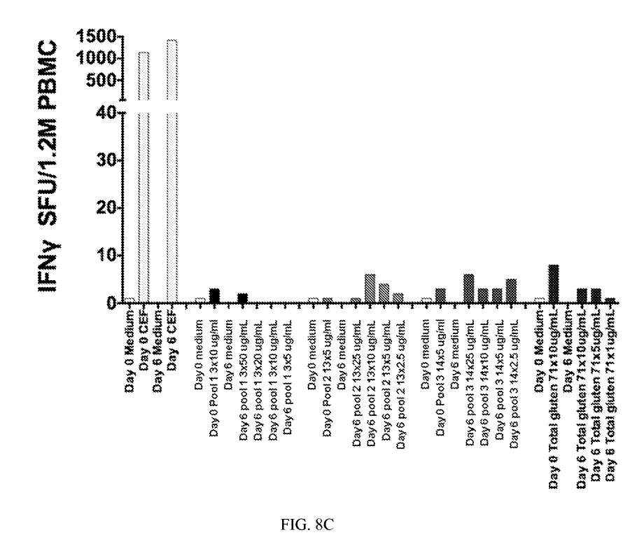

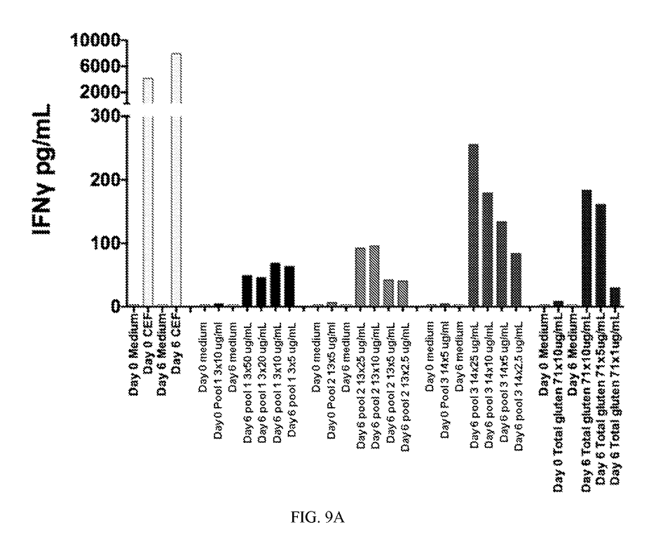

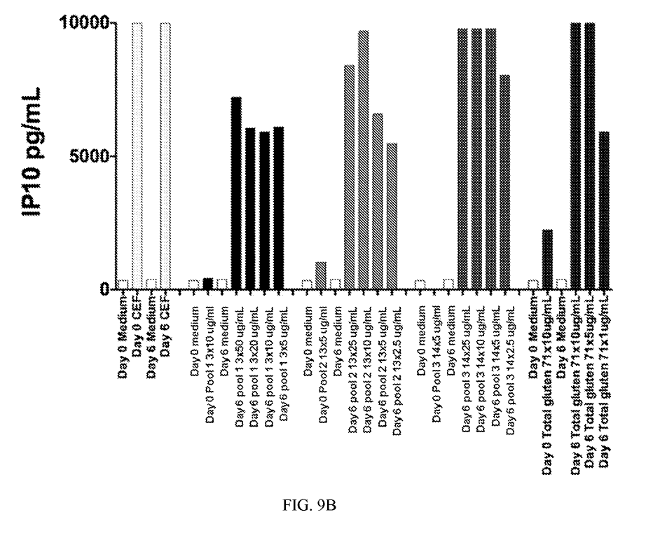

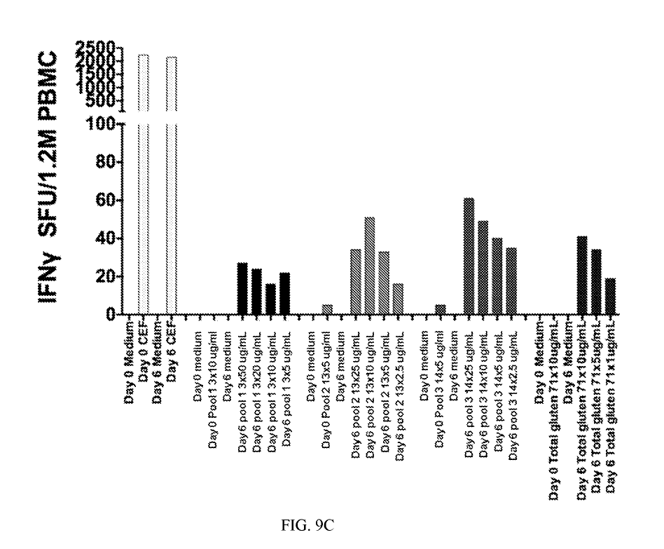

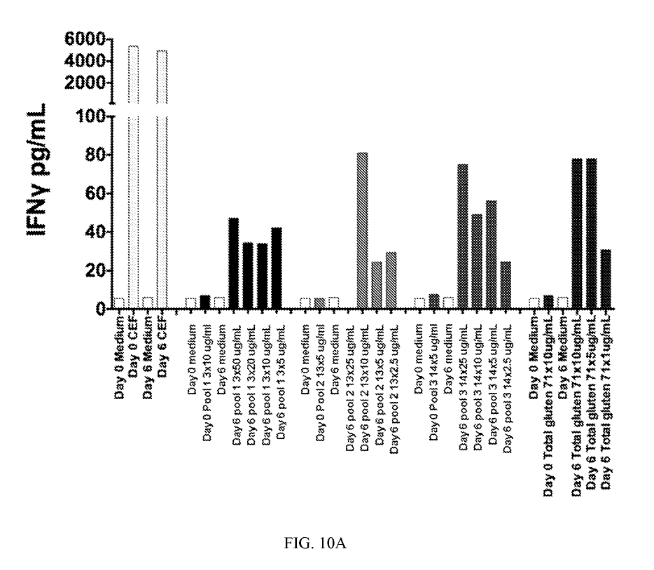

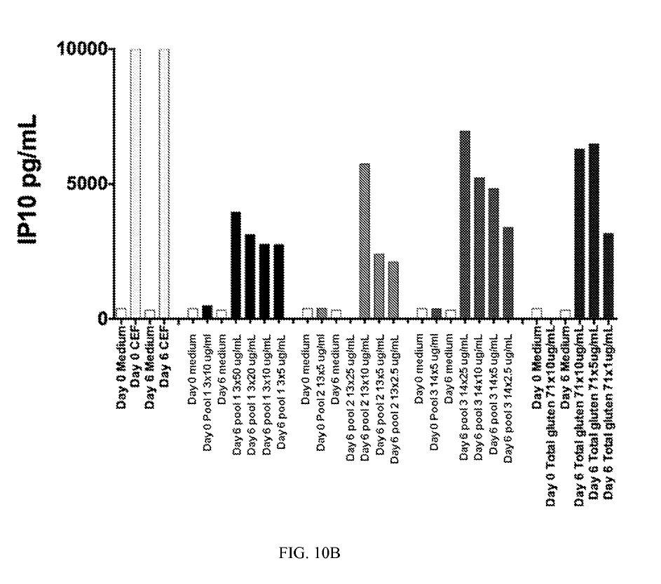

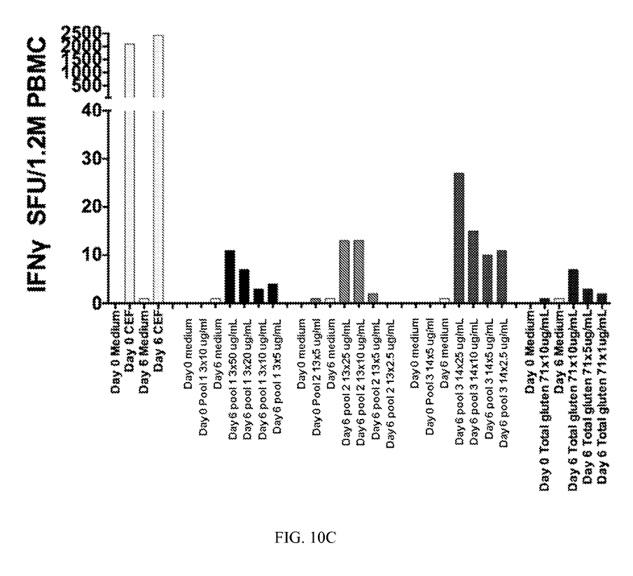

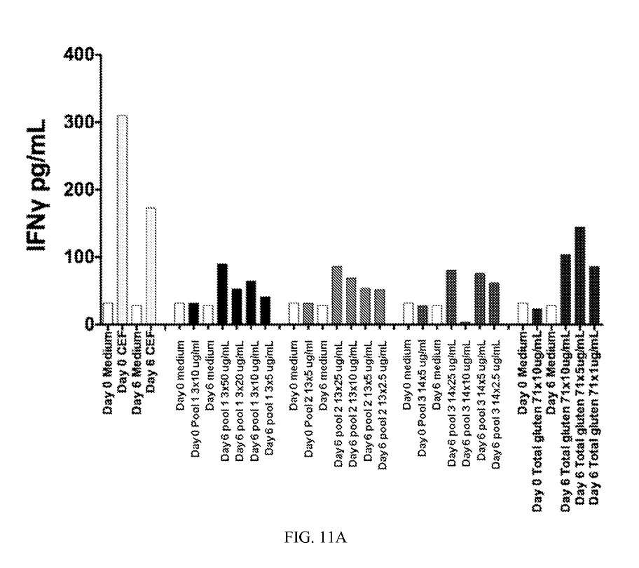

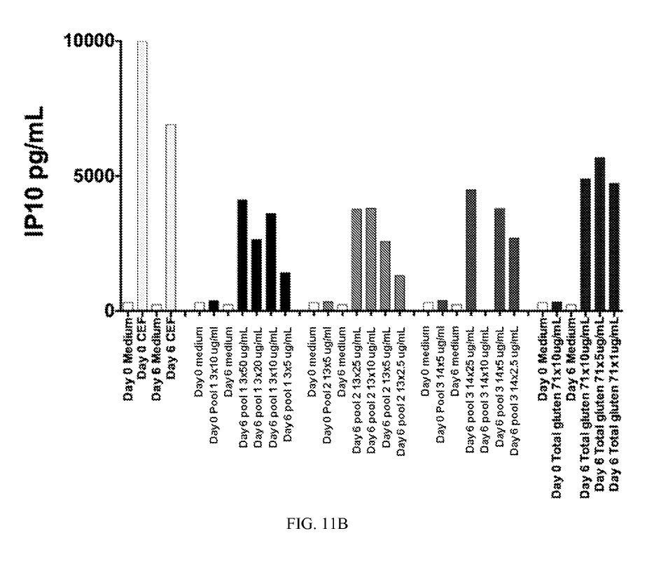

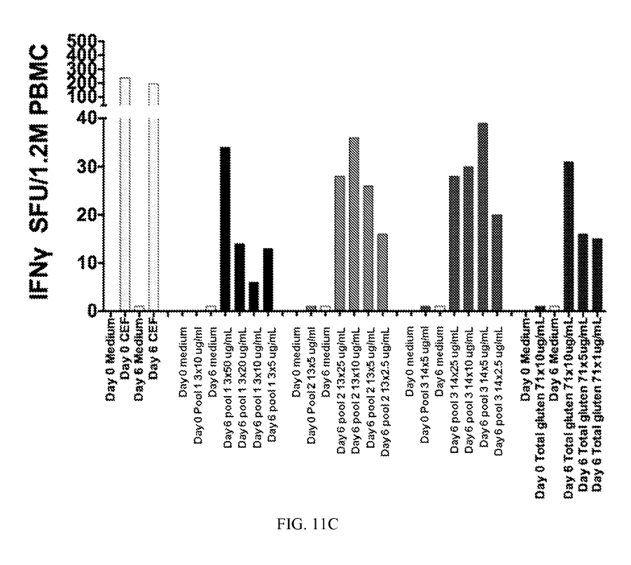

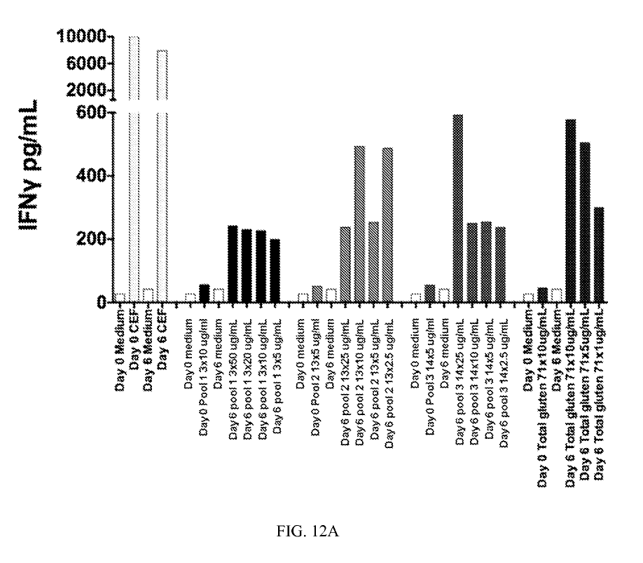

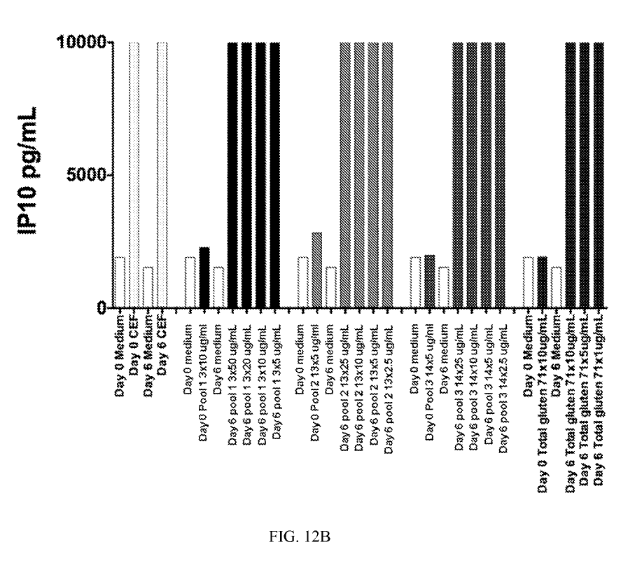

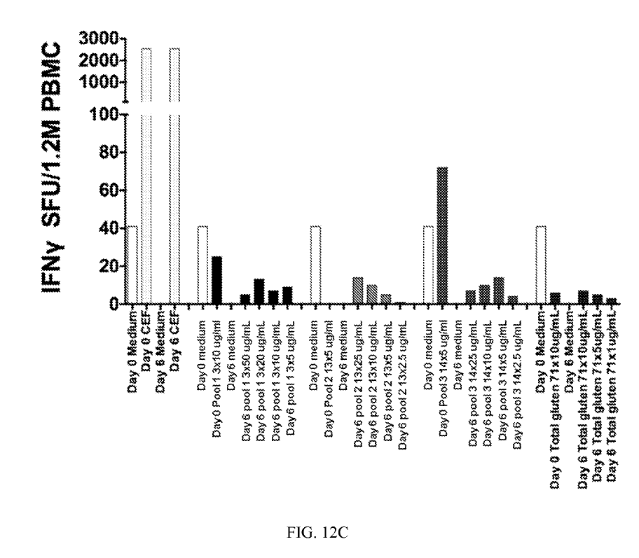

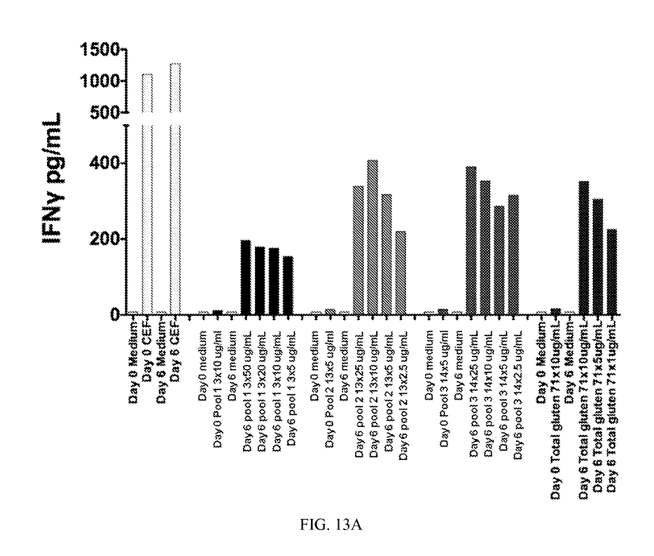

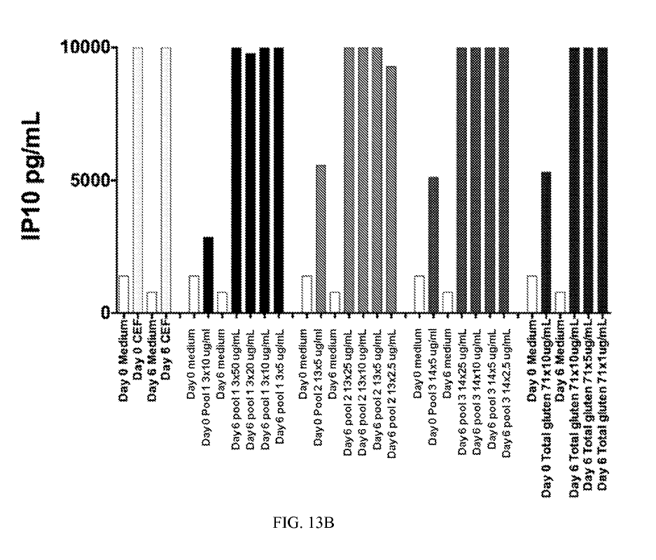

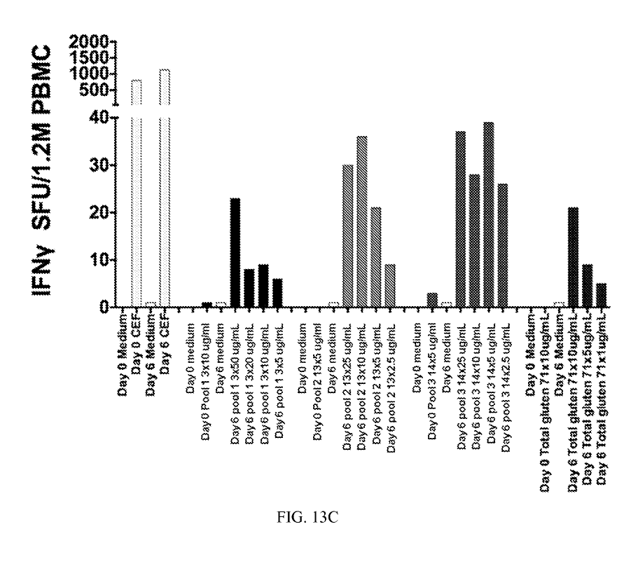

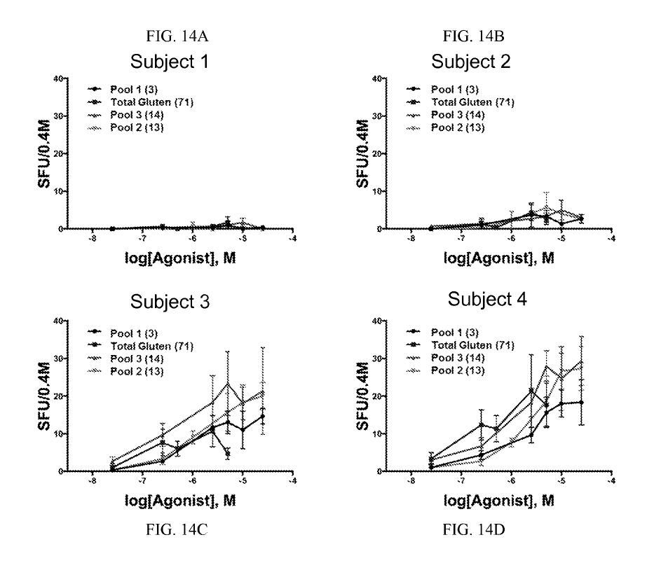

Furthermore, elevated IP-10 levels were detected in blood collected from HLA-DQ2.5+ celiac disease patients on a gluten free diet without prior gluten challenge following incubation with pools 1, 2, and 3 (FIGS. 4-16). These results were unexpected, as it was previously thought that a minimum of 3 days of oral gluten challenge was required to mobilize T cells to a degree that could be detected with a blood assay (see, e.g., Anderson et al. T cells in peripheral blood after gluten challenge in coeliac disease. Gut. 2005 September; 54(9):1217-23). These results suggest that levels of IP-10 can be used to diagnose Celiac disease in the absence of an oral gluten challenge, with a shortened gluten challenge (e.g., 1 or 2 day oral gluten challenge), or in subjects that have low or insignificant levels of IFN-γ.

TABLE 1
Whole blood secretion of IFNγ pre- and post-gluten challenge

| | | | | Day 6 |
|---|---|---|---|---|
| | Day 0 | Day 0 | Day 6 | Pool 1 minus |
| IFNγ | Ratio Pool | Pool 1 minus | Ratio Pool | Medium |
| MAGPIX | 1:Medium | Medium (pg/mL) | 1:Medium | (pg/mL) |
| Minimum | 0.9969 | −0.1 | 0.72 | −259 |
| 25% | 1.086 | 1.205 | 0.9014 | −17.58 |
| Percentile | | | | |
| Median | 1.265 | 4.625 | 5.53 | 50.77 |
| 75% | 1.689 | 21.01 | 29.82 | 185.6 |
| Percentile | | | | |
| Maximum | 2.6 | 143 | 228.6 | 728.2 |

Day 0 = prior to oral gluten challenge,
Day 6 = 6 days after commencing a 3-day gluten challenge.

TABLE 2
Whole blood secretion of IP10 pre- and post-gluten challenge

| | Day 0 | Day 0 | Day 6 | Day 6 Pool 1 |
|---|---|---|---|---|
| IP10 | Ratio Pool | Pool 1 minus | Ratio Pool | minus Medium |
| MAGPIX | 1:Medium | Medium (pg/mL) | 1:Medium | (pg/mL) |
| Minimum | 1.192 | 65.26 | 3.648 | 1230 |
| 25% | 1.235 | 100.7 | 5.861 | 2168 |
| Percentile | | | | |
| Median | 1.536 | 274.7 | 13.00 | 6998 |
| 75% | 1.876 | 608.8 | 17.99 | 9333 |
| Percentile | | | | |
| Maximum | 3.186 | 1453 | 26.15 | 9618 |

Day 0 = prior to oral gluten challenge,
Day 6 = 6 days after commencing a 3-day gluten challenge.

### Example 2

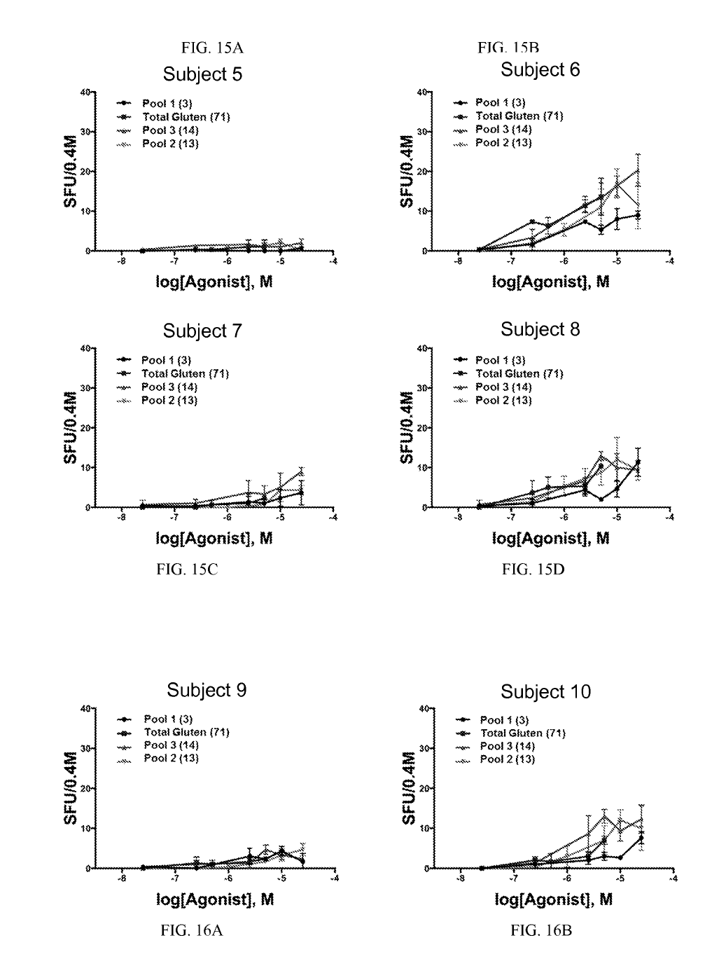

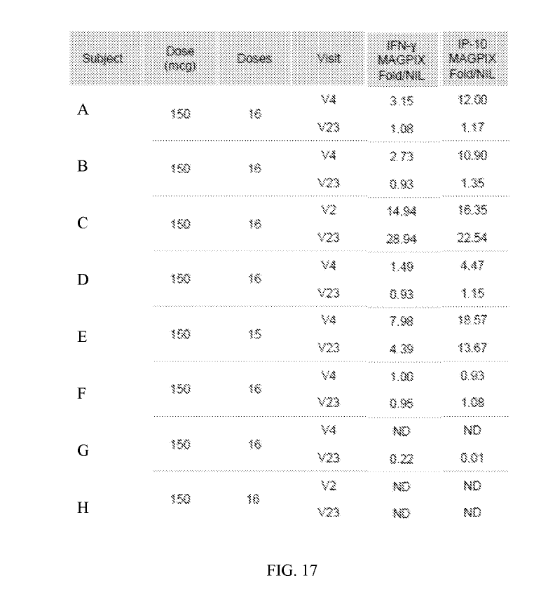

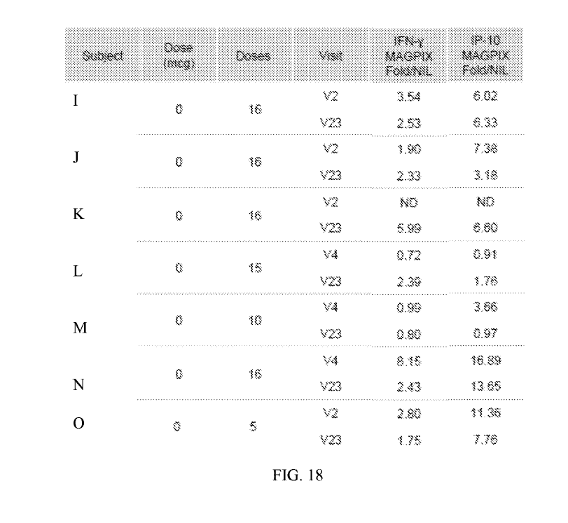

Data from subjects with HLA-DQ2.5+ Celiac disease on a gluten-free diet treated with 150 micrograms of peptide pool 1 were subjected to an oral gluten challenge before the first dose of treatment and after the last dose of treatment. IP-10 was then measured by MAGPIX in whole blood samples from each subject after the whole blood was contacted with peptide pool 1 or a negative control. A summary of the IP-10 measurements is shown in FIG. 17, compared to corresponding IFN-γ MAGPIX. In general, the fold change was high (>4) prior to the first dose and low after the last dose (<2) using the IP-10 assay. This fold change difference between first dose and last dose was not observed in subjects treated with placebo (FIG. 18). These data further confirm that IP-10 can be used to identify subjects with Celiac disease.

### Example 3

### Peptide Compositions and Detection Assays for Gluten-Reactive T Cells in Celiac Disease

Peptide selection is important to the design of epitope-specific immunotherapy (ESIT) and epitope-specific immunodiagnostics (ESID).¹ ESIT's and ESID's require selection of peptides with epitopes that are recognized by a substantial proportion of the CD4+ T cells responsible for pathology.

The frequency and hierarchy of gluten-specific T cells in vivo can be quantified in overnight IFNγ ELISpot assays using freshly isolated peripheral blood mononuclear cells (PBMC) collected after patients undergo oral gluten challenge.^{2,3} Optimizing minimal peptide compositions generally requires not only quantitative assays, but also establishing that epitopes are mostly non-redundant, i.e. that each peptide targets distinct T cell populations that together consistently account for a substantial proportion of pathogenic T cells in patients. In celiac disease, circulating gluten-reactive T cells are extremely rare and so far have not been detected by quantitative cytokine release assays except after oral gluten challenge.² However, oral challenge with wheat, barley, and rye reactivates gluten-reactive T cell populations with subtly different specificities that allowed selection of the three peptides included in P3.

The three peptides in pool P3 (containing Peptides 1, 2, and 3 in Table 6) constitute at least five, mostly non-redundant HLA-DQ2.5-restricted epitopes. IFNγ ELISpot studies using blood collected after oral gluten challenge indicate that ex vivo T cell responses to an optimal concentration of P3 (3×50 μg/mL) is about ⅔ of that elicited by peptic digests of semi-purified gliadin, hordein or the most active secalin fraction (co-secalin) pre-treated with transglutaminase.³

In vitro studies with T cell clones specific for P3 indicate that the five defined epitopes in P3 are relatively non-redundant, and recognize over two-thirds of the 90 wheat, barley and rye prolamin-derived peptides confirmed to be T-cell stimulatory in HLA-DQ2.5+ CD patients.³ However, there are some relatively potent T-cell stimulatory gluten-derived peptides that are not recognized by T cell clones specific for P3. This suggests that additional peptides could be added to P3 to increase the size of the responding gluten-reactive T cell population present in HLA-DQ2.5+ celiac disease patients.

Two strategies would be expected to increase the population of responding T cells: (1) selecting non-redundant HLA-DQ2.5 epitopes not already covered by P3, and (2) selecting peptides with gluten-derived epitopes restricted by celiac disease-associated HLA-DQ molecules apart from HLA-DQ2.5 (e.g. HLA-DQ8, HLA-DQ2.2 or transdimers formed between HLA-DQA and DQB chains of HLA-DQ2.5 and DQ8). The prevalence of HLA-DQ2.5 in patients confirmed to have celiac disease is typically about 90% (Table 3). But HLA-DQ2.2 or HLA-DQ8 is also present in about ⅓ of HLA-DQ2.5+ patients, and in patients not carrying HLA-DQ2.5, HLA-DQ8 or HLA-DQ2.2 are usually present.

TABLE 3
HLA-DQ genetics in celiac disease

| | %⁴ | %⁵ | %⁶ | %⁷ |
|---|---|---|---|---|
| HLA-DQ^{2.5+/any} | 91 | 91 | 94 | 88 |
| HLA-DQ^{2.5+/2.5+} | 13 | | 20 | |
| HLA-DQ^{2.5+/2.2+} | 28 | | 20 | |
| HLA-DQ^{2.5+/8+} | 4 | 9 | | |
| HLA-DQ^{2.5-/8+} | 7 | 5 | 2 | 6 |
| HLA-DQ^{2.5-/8-/2.2+} | 4 | 2 | 1 | |
| HLA-DQ^{2.5-/8+/2.2+} | 2 | | | |
| HLA-DQ^{2.5-/8-/2.2+} | 2 | | | 4 |
| HLA-DQ^{2.5-/8+/2.2-} | 5 | | | |

The hierarchy of wheat gluten peptides recognized by circulating T cells in HLA-DQ8+ 2.5-patients after wheat challenge has been reported,⁸ and is relatively consistent with in vitro studies using intestinal T cell lines.⁹⁻¹¹ Amongst the peptides recognized by circulating T cells in HLA-DQ8+ patients, three HLA-DQ8-restricted epitopes are efficiently presented by transdimers of HLA-DQ2.5 and 8.^{11,12} Only one HLA-DQ2.2-restricted epitope has been reported,¹³ but earlier the same sequence had also been claimed to activate T cell clones from HLA-DQ2.5+ donors.¹⁴ As described herein, addition of further epitopes to those in the three peptides in P3 was expected to increase T cell responses in patients who have HLA-DQ2.2 and/or 8 whether or not they also carry HLA-DQ2.5.

In principle, the activity of new peptide pools compared to P3 is readily measurable using optimal concentrations of peptides in ex vivo cytokine release assays with fresh polyclonal T cells circulating in blood after oral gluten challenge.

Quantifying the proportion of the pathogenic T cell population targeted is also likely to predict therapeutic efficacy and diagnostic accuracy of peptide compositions. In celiac disease, all pathogenic T cells are specific for gluten and the specificities of gluten-reactive T cells circulating after oral challenge with wheat, barley and rye have been exhaustively mapped.³

Although IFNγ ELISpot using fresh PBMC from patients following oral gluten challenge has been the mainstay of studies mapping and quantifying the importance of epitopes for circulating gluten-reactive T cells, other assay formats may be more sensitive for detection of rare antigen-specific T cells.¹⁵ The median frequency of effector memory T cells in blood from patients with treated celiac disease that are stained by tetramers for either the DQ2.5-gliaα1a or DQ2.5-gliaα2 epitopes is 5 per million CD4 T cells and in untreated patients 15 per million.¹⁶ The frequency of CD4 T cells in blood is 0.30-1.50 million/mL, implying that the frequency of T cells specific for Peptide 1, which contains the DQ2.5-gliaα1a or DQ2.5-gliaα2 epitopes (Peptide 1), is in the range 1.5-7.5/mL in treated celiac disease patients. Peptide 1 stimulates IFNγ secretion by cognate T cells, and in blood both monocytes and neutrophils are known to secrete the chemokine IP-10 when incubated with IFNγ.^{17,18} Monocytes and neutrophils are abundant in blood (0.20-0.90 million/mL and 2.09-5.97 million/mL, respectively). IP-10 plays an important role in the recruitment of T cells and monocytes to sites of inflammation.¹⁹ In principle, IP-10 in whole blood incubated with gluten peptides could be a sensitive, and more robust biomarker than IFNγ for the presence of activated gluten-specific T cells. Whole blood release of IP-10 is as sensitive as IFNγ for detection of T cells specific for Peptide 1 and Peptide 2 in celiac disease patients after oral gluten.¹⁵ Whole blood release of IP-10 may also be more sensitive than IFNγ to support the diagnosis of mycobacterium tuberculosis infection.^{20,21}

The primary objective of the current study was to test whether adding peptides to P3 could increase IFNγ and IP-10 secretion in cytokine release assays using fresh blood or PBMC. A secondary objective was to compare the sensitivity of whole blood IFNγ and IP-10 release assays for detection of gluten-reactive T cells before and after oral gluten challenge.

### Methods

### Clinical

Ten HLA-DQ2.5+ adults with celiac disease diagnosed according to the National Institutes of Health Consensus Statement 2004 were enrolled (Table 4).²² Subjects were required to have followed gluten-free diet for at least one year and without known gluten exposures within the previous two-months. Celiac disease-specific serology was also required to be no greater 50% above the upper level of normal, but in fact all subjects showed transglutaminase (tTG)-IgA (INOVA 704605 QUANTA Lite® R h-tTG IgA or 708760 QUANTA Lite® h-tTG IgA, San Diego, Calif. 92131) and deamidated gliadin peptide (DGP)-IgG (704520 QUANTA Lite® Gliadin IgG II (DGP)) within the normal range. Full inclusion and exclusion criteria are described in Table 5. Each of 3 consecutive days subjects consumed 3 cookies that were prepared from approximately 4.5 g wheat gluten, 3 g barley flour protein, and 1.5 g rye flour protein. Blood was collected before and six days after commencing the oral challenge. Blood was collected using a 21G butterfly needle directly into 10 mL lithium heparin tubes (BD Vacutainer® Heparin tube #367880) and QuantiFERON® NIL (0591-0205, Cellestis Ltd., Chadstone VIC 3148 Australia) and QuantiFERON® MITOGEN (0593-0201, Cellestis Ltd.).

TABLE 4
HLA-DQ and serology status of subjects

| Subject | | | HLA-DQA1 | HLA-DQB1 | HLA- | HLA- | tTG- | DGP- |
|---|---|---|---|---|---|---|---|---|
| ID# | Sex | Age | alleles | alleles | DQ#1 | DQ#2 | IgA¹ | IgG¹ |
| 78 | F | 51 | | 02:01, | 2.5 | 6 | <5* | <20 |
| | | | | 06:03 | | | | |
| 115 | F | 49 | | 02:01, | 2.5 | 2.2 | <5* | <20 |
| | | | | 02:02 | | | | |
| 120 | F | 41 | | 02:01/02:02 | 2.5 | 2.2 | 1.2 | 2 |
| 160 | M | 45 | 05:01 | 02:01, | 2.5 | 6 | 5.2 | 6 |
| | | | | 06:09 | | | | |
| 205 | F | 48 | | 02:01, | 2.5 | 2.2 | <2* | 5 |
| | | | | 02:02 | | | | |
| 232 | M | 47 | 02:01, 05:01 | 02:01, | 2.5 | 2.2 | 2.6 | 2 |
| | | | | 02:02 | | | | |
| 529 | M | 43 | 01:02/08/09, | 02:01, | 2.5 | 6 | 7.6 | 4 |
| | | | 05:01 | 06:02G | | | | |
| 553 | M | 36 | 01, 05 | 02, 06 | 2.5 | 6 | 12.5 | 3 |
| 586 | F | 42 | 01, 05 | 02, 06 | 2.5 | 6 | 5.3 | 1 |
| 617 | F | 48 | | 02, 0302 | 2 | 8 | 5.7 | 4 |

¹Upper limit of normal (ULN) range is 20 units unless marked by * in which case the ULN is 5.

TABLE 5
Entry Criteria

Inclusion criteria:
a. Aged between 18 and 50 years.
b. HLA-A1*05 and HLA-B1*02 present (HLA-2.5+).
c. Celiac disease without Type-1 diabetes. Celiac disease diagnosed according to
National Institutes of Health Consensus Statement 2004 (Department of Health and Human
Services, 2004): small bowel histology showing at least villous atrophy, and serology
showing elevated transglutaminase IgA or abnormal endomysial immunofluorescence while
gluten is being regularly consumed.
d. Following strict gluten free diet.
e. Willing to consume an amount of gluten equivalent to approximately 4 slices of bread
daily for three days.
f. Provide written informed consent.
Exclusion criteria
a. Individual has not been prescribed and/or has not followed a GFD for at least 12
months or has had known gluten exposure within two months prior to screening.
b. Subject with elevation in transglutaminase [tTG]-IgA, deamidated gliadin peptide
[DGP]-IgA or IgG to a level ≧50% above upper limit of normal range for that assay.
c. Individual has uncontrolled complications of celiac disease or unstable autoimmune
disease which, in the opinion of the investigator, would impact the immune response or pose
an increased risk to the patient.
d. Individual has had treatment with systemic biological agents (e.g., adalimumab,
etanercept, infliximab, certolizumab pegol) less than six months prior to screening.
e. Individual has taken systemic immunomodulatory agents (e.g., azathioprine,
methotrexate) less than 30 days prior to screening.
f. Human immuno-deficiency virus-1 and -2 (HIV1 + 2) infection or active, untreated
hepatitis B virus (HBV) or hepatitis C virus (HCV) infection.

### Peptides

The P3 pool was prepared in sterile normal saline from acetate salts of Peptides 1, 2 and 3 (purities >98%; CSBio Menlo Park, Calif. 94025) to yield a stock equimolar solution (0.7 mL/vial stored at −20° C.) with constituent peptides at a concentration of 15.6 mM (MicroTest Laboratories, Inc.; Agawam MA 01001) (Table 6). The P14 pool (Table 6) included trifluoroacetate (TFA) salts of 14 peptides between 14 and 19 amino-acids (Mᵣ median: 1801.6, range: 1601.7-2228.6 g/mol) (Pepscan Presto By, 8243 RC Lelystad, The Netherlands). Identities of constituent peptides were confirmed by LC/MS. Median purity assessed by HPLC was 97.4% (range: 95.0-99.8%). P14 was constituted as a lyophilized mixture in vials containing 0.2 μmol of each peptide that was stored at −20° C. until being dissolved directly in sterile normal saline yielding 5 mM per peptide. The P13 (Table 6) stock solution (13×1.556 mM) consisted of P3 diluted by the addition of the other 10 constituent peptides (Pepscan) individually dissolved in normal saline. P71 was prepared as a PepMix™ Peptide Pool from TFA salts of 71 individual peptides 14 to 19 amino acids in length (Mᵣ median: 1688.85, range: 1423.51-2229.48 g/mol) (JPT Peptide Technologies GmbH, 12489 Berlin, Germany). The identity of constituent peptides was confirmed by LC/MS, and median purity assessed by HPLC was 86% (range: 71-98.8%). Individual vials of lyophilized P71 containing 0.1 mg of each peptide were stored at 20° C. until being first dissolved in dimethylsulfoxide (DMSO) (10 mg/mL per peptide) then diluted to 1 mg/mL in sterile normal saline. The CEF pool of 23 peptides consisting of MHC class I-restricted T-cell epitopes from human cytomegalovirus, Epstein Barr virus and influenza virus was purchased from Mabtech (#3615-1; Nacka Strand, Sweden). Each vial of CEF contained 0.1 mL of a 10% DMSO aqueous solution with each peptide at a concentration of 0.2 mg/mL (individual peptide purities were >95%) that was stored at −20° C. Individual gluten peptides were dissolved directly in normal saline to 5 mM. Individual peptides and pools were further diluted in PBS and DMSO to achieve a DMSO concentration of 1%, and 10× the final assay concentration of peptide. All peptides referred to as “Peptide X” discussed in Example 3 refer to those in Table 6.

TABLE 6
Peptide Pools

| | P3 | P13 | P14 | P13alt | | HLA-DQ | Epitope |
|---|---|---|---|---|---|---|---|
| Peptide | pool | pool | pool | pool | Sequence | Restriction | sequences |
| 1 | Present | Present | Absent | Present | (pE)LQPFPQPE | DQ2.5 | PFPQPELPY |
| | | | | | LPYPQPQ- | | (SEQ ID NO: 1), |
| | | | | | amide (SEQ ID | | PQPELPYPQ |
| | | | | | NO: 265) | | (SEQ ID NO: 2) |
| 2 | Present | Present | Present | Present | (pE)QPFPQPEQ | DQ2.5 | PFPQPEQPF |
| | | | | | PFPWQP-amide | | (SEQ ID NO: 3), |
| | | | | | (SEQ ID NO: | | PQPEQPFPW |
| | | | | | 266) | | (SEQ ID NO: 4) |
| 3 | Present | Present | Absent | Present | (pE)PEQPIPEQP | DQ2.5 | EQPIPEQPQ |
| | | | | | QPYPQQ-amide | | (SEQ ID NO: 5), |
| | | | | | (SEQ ID NO: | | PIPEQPQPY |
| | | | | | 267) | | (SEQ ID NO: 6) |
| 4 | Absent | Present | Present | Present | (pE)QPFPQPEQ | DQ2.5/2.5 + 8/8 | PFPQPEQPI |
| | | | | | PIPVQPEQS- | | (SEQ ID NO: 7), |
| | | | | | amide (SEQ ID | | PQPEQPIPV |
| | | | | | NO: 268) | | (SEQ ID NO: 8), |
| | | | | | | | EQPIPVQPE |
| | | | | | | | (SEQ ID NO: 9) |
| 5 | Absent | Present | Present | Present | (pE)QPFPQPEQ | DQ2.5/2.5 + 8/8 | PFPQPEQPT |
| | | | | | PTPIQPEQP- | | (SEQ ID NO: 10), |
| | | | | | amide (SEQ ID | | PQPEQPTPI |
| | | | | | NO: 269) | | (SEQ ID NO: 11), |
| | | | | | | | EQPTPIQPE |
| | | | | | | | (SEQ ID NO: 12) |
| 6 | Absent | Present | Present | Present | (pE)QPFPQPEQ | DQ2.5/2.5 + 8/8 | PFPQPEQPF |
| | | | | | PFPLQPEQP- | | (SEQ ID NO: 3), |
| | | | | | amide (SEQ ID | | PQPEQPFPL |
| | | | | | NO: 270) | | (SEQ ID NO: 13), |
| | | | | | | | EQPFPLQPE |
| | | | | | | | (SEQ ID NO: 14) |
| 7 | Absent | Present | Present | Absent | (pE)QPFPQPEQ | DQ2.5 | PFPQPEQPF |
| | | | | | PFSQQ-amide | | (SEQ ID NO: 3), |
| | | | | | (SEQ ID NO: | | PQPEQPFSQ |
| | | | | | 271) | | (SEQ ID NO: 40) |
| 8 | Absent | Present | Present | Absent | (pE)PQPYPEQP | DQ2.5 | PYPEQPQPF |
| | | | | | QPFPQQ-amide | | (SEQ ID NO: 22) |
| | | | | | (SEQ ID NO: | | |
| | | | | | 272) | | |
| 9 | Absent | Present | Present | Present | (pE)SGEGSFQP | DQ8/2.5 + 8/8 | EGSFQPSQE |
| | | | | | SQENPQ-amide | | (SEQ ID NO: 15) |
| | | | | | (SEQ ID NO: | | |
| | | | | | 273) | | |
| 10 | Absent | Present | Present | Present | (pE)GQQGYYP | DQ2.5/2.5 + 8/8 | QGYYPTSPQ |
| | | | | | TSPQQSG- | | (SEQ ID NO: 16) |
| | | | | | amide (SEQ ID | | |
| | | | | | NO: 274) | | |
| 11 | Absent | Present | Present | Present | (pE)PEQPEQPF | DQ2.5/2.5 + 8/ | EQPEQPFPE |
| | | | | | PEQPQQ-amide | 8/2.2 + 8 | (SEQ ID NO: 17), |
| | | | | | (SEQ ID NO: | | EQPFPEQPQ |
| | | | | | 275) | | (SEQ ID NO: 41) |
| 12 | Absent | Present | Present | Absent | (pE)QPFPEQPE | DQ2.5 | PFPEQPEQI |
| | | | | | QIIPQQP-amide | | (SEQ ID NO: 42) |
| | | | | | (SEQ ID NO: | | |
| | | | | | 276) | | |
| 13 | Absent | Present | Present | Present | (pE)QPPFSEQE | DQ2.2 | PFSEQEQPV |
| | | | | | QPVLPQ-amide | | (SEQ ID NO: 18) |
| | | | | | (SEQ ID NO: | | |
| | | | | | 277) | | |
| 14 | Absent | Absent | Present | Absent | (pE)PEQPFPEQ | DQ2.5 | EQPFPEQPI |
| | | | | | PIPEQPQPYP- | | (SEQ ID NO: 20), |
| | | | | | amide (SEQ ID | | PFPEQPIPE |
| | | | | | NO: 278) | | (SEQ ID NO: 21), |
| | | | | | | | EQPIPEQPQ |
| | | | | | | | (SEQ ID NO: 5), |
| | | | | | | | PIPEQPQPY |
| | | | | | | | (SEQ ID NO: 6) |
| 15 | Absent | Absent | Present | Present | (pE)QPYPQPEL | DQ2.5 | PYPQPELPY |
| | | | | | PYPQPQ-amide | | (SEQ ID NO: 19), |
| | | | | | (SEQ ID NO: | | PQPELPYPQ |
| | | | | | 279) | | (SEQ ID NO: 2) |
| 16 | Absent | Absent | Present | Absent | (pE)QPFPQPEL | DQ2.5 | PFPQPELPY |
| | | | | | PYPYPQ-amide | | (SEQ ID NO: 1), |
| | | | | | (SEQ ID NO: | | PQPELPYPY |
| | | | | | 280) | | (SEQ ID NO: 43) |
| 17 | Absent | Absent | Absent | Present | (pE)PQEQPFPE | DQ2.5 | EQPFPEQPI |
| | | | | | QPIPEQP-amide | | (SEQ ID NO: 20), |
| | | | | | (SEQ ID NO: | | PFPEQPIPE |
| | | | | | 281) | | (SEQ ID NO: 21) |
| 18 | Absent | Absent | Absent | Present | (pE)QPQPYPEQ | DQ2.5 | PQPYPEQPQ |
| | | | | | PQPFPQQ-amide | | (SEQ ID NO: 23), |
| | | | | | (SEQ ID NO: | | PYPEQPQPF |
| | | | | | 282) | | (SEQ ID NO: 22) |

Peptide = Peptide identifier,
(pE) = pyroglutamate,
Present = present in the pool listed in the top row (P3, P13, P14, or P13alt),
Absent = not present in the pool listed in the top row (P3, P13, P14, P13alt).

### Cytokine Release Assays

### 96-Well Plate Whole Blood Multiplex Cytokine Release

Gluten peptide solutions for addition to whole blood were arranged according to standardized templates in sterile 96-well U-bottom plates (30 μL/well). Plates were sealed with adhesive ELISA plate cover slips before the plastic lid was replaced and being frozen at −80° C. Whole blood assay (WBA) “medium” (1% DMSO in 90% PBS and 9% normal saline), mitogen (PHA-L, Sigma-Aldrich #L2769; St Louis Mo.) 100 μg/ml in WBA medium, and CEF diluted to 10 μg/ml in PBS with a final concentration of 1% DMSO were frozen separately in sterile cryovials. 96-well plates and cryovials with frozen incubation solutions were shipped on dry ice to the clinical site where blood was collected and incubations performed. Solutions were thawed at room temperature for 10 min just before being added to whole blood. 96-well plates were thawed while being centrifuged at 300 g to avoid surface condensation. A multi-channel pipette was used to efficiently transfer 25 μL of peptide solutions from the original 96-well plate to corresponding wells in a fresh sterile 96-well U-bottom plate. Once incubation solutions had been added, 225 μL of whole blood was added to each well and the plate transferred to incubate for 24 h at 37° C. 5% CO₂. DMSO was present in all assays at a final concentration of 0.1%, the highest concentration tested that did not reduce antigen-stimulated whole blood IFNγ or IP-10 secretion (data not shown). Individual peptides were incubated at a final assay concentration of 51.1M. Final concentrations of individual peptides assessed in P3 was 0.05-50 μg/mL, and in P14 and P13 pools 0.025-25 μM. The P71 pool was tested at between 0.005-10 pg/mL, the highest tested concentration being the maximal possible not exceeding 0.1% DMSO. Peptide pools, mitogen, and medium only were assessed in triplicate wells, and on Day-6 individual peptides were assessed in duplicate wells. Incubations were terminated after 24 h, and 96-well plates centrifuged at 300 g for 10 min. A total of approximately 90 to 120 μL of plasma was removed from each well taking care to avoid red cell contamination, and transferred into corresponding wells of two further 96-well plates (one with 30 μL/well and the residual in the second plate). Plasmas were frozen at −80° C. and later shipped on dry ice to the central lab where IP-10 and IFNγ multiplex bead assays were performed according to manufacturer's instructions using 25 μL plasma per well (Milliplex® MAP Human Cytokine/Chemokine Magnetic Bead Panel #HCYTOMAG-60k-02; EMD Millipore Corp., Billerica, Mass. 01821) and analysed with the Luminex® MAGPIX® System xPONENT® software (Luminex Corporation, Austin Tex. 78727). Plasma cytokine levels for each assay condition were expressed as the mean analyte concentration of each of the three replicate incubations. Laboratory staff were unaware of the arrangement of peptide solutions incubated with during assay setup and plate counting.

### In-Tube Whole Blood IFNγ Secretion

Aliquots of PBS, P3 peptides diluted in PBS (final concentration in blood 3× 50 or CEF to a final concentration 1 μg/mL were prepared in sterile cryovials (0.11 mL/vial) and stored frozen (−80° C.) until immediately before use. P3, CEF and two PBS aliquots were drawn up in separate sterile 0.3 mL insulin syringes with attached 29G×½″ needle (Terumo, SS*30M2913; Elkton Md. 21921). P3, CEF and one PBS aliquot was injected through the stoppers of three separate QuantiFERON® NIL tubes already containing 1 mL blood, and one aliquot of PBS was injected into a QuantiFERON® MITOGEN tube already containing 1 mL blood. All four tubes containing blood and incubation solution were gently inverted ten-times and transferred to incubate at 37° C. 5% CO₂. After for 24 h, tubes were centrifuged 300 g 10 min. Plasma was separated and equal volumes placed in two 1.5 mL cryovials. Frozen plasmas were transferred to the central lab where IFNγ levels were measured in triplicate 50 μL samples by ELISA (MABtech Human IFN-γ ELISA development kit HRP, 3420-1H-6; capture mAb 1-D1K). IFNγ responses were considered elevated if levels were more than 7.2 pg/mL greater than in the medium only tube, and the ratio between IFNγ levels in the NIL tube with P3 or CEF to the NIL tube with PBS only (stimulation index, SI) was >1.25.

### ELISpot Assay

Overnight IFNγ ELISpot assays (Human IFN-γ ELISpotPRO kit, transparent, ALP; Mabtech AB, #3420-2APT-10) were performed using PBMC freshly separated from heparinized blood diluted 1:1 in PBS with 2% fetal bovine serum (Stemcell Technologies #07905; Vancouver, BC, V5Z 1B3, Canada) overlaid on density gradient medium (Ficoll-Paque™ PLUS; GE Healthcare Lice Sciences #:17-1440-02) in SepMate™-50 tubes (Stemcell Technologies #15460). PBMC (8 million/mL) were resuspended in serum-free medium with gentamicin and phenol red (X-VIVO™15; Lonza, Walkersville Mass. 21793). PBMC (0.4 million/50 pt/well) and incubated with 50 μL 80% X-vivo15, 0.2% DMSO and 20% PBS in three triplicate wells (“medium only”), or with 50 μL PHA-L 20 μg/ml or peptide at 2× final concentration in 80% X-vivo15, 0.2% DMSO and 20% PBS. Peptide solutions to be added to individual ELISpot wells were prepared in 96-well U-bottom plates (60 pt/well) that were then sealed with adhesive ELISA plate cover slips before replacing the plastic lid and being frozen at −80° C. 96-well plates containing peptide solutions were thawed at room temperature for 10 min while being centrifuged at 300 g immediately prior to being added to ELISpot wells. Peptide pools and mitogen were assessed in triplicate wells, and individual peptides were assessed in duplicate wells. X-VIVO™15 (50 μL) was incubated with 80% X-vivo15, 0.2% DMSO and 20% PBS (50 μL) in triplicate wells (“no PBMC” control). A Zeiss automated ELISpot counter was used to determine spot forming units (SFU) per well (Zellnet Inc., Fort Lee, N J 07024). Laboratory staff were unaware of the arrangement of peptide solutions in ELISpot wells during assay setup and plate counting.

### Results

### Design and Preparation of Gluten Peptide Pools

There is presently no functional assay that enumerates all gluten-reactive T cells relevant to celiac disease. Wheat gluten is a variable mixture of aqueous insoluble proteins that requires digestion by proteases and deamidation to be efficiently activate T cells in patients with celiac disease. Hordeins from barley and secalins from rye are also complex mixtures of proteins closely related to wheat gluten that harbor potent CD4+ T cell epitopes that are not represented in wheat gluten. Furthermore gluten contains other proteins such as amylase trypsin inhibitors (ATI's) that activate innate immune cells and may compromise interpretation of functional immuno-assays.²³ Whole protein and mixtures of overlapping peptide spanning a protein antigen's primary sequence both efficiently reactivate recall CD4+ and CD8+ T-cell responses to CMV and HIV antigens in vitro.^{24,25} A pool of synthetic peptides including a comprehensive set of epitopes implicated in celiac disease could be used to stimulate virtually all gluten-reactive T-cells.

After comprehensive, unbiased epitope screening, only 90 14-16mer peptides were confirmed to be stimulatory for T cells circulating in blood after oral challenge with wheat, barley or rye in HLA-DQ2.5+ patients with celiac disease.³ A pool consisting of 71 peptides (P71) was designed to include each of the core immunogenic sequences in the 90 immunogenic peptides implicated in HLA-DQ2.5+ celiac disease as well as three HLA-DQ8- and one HLA-DQ2.2-restricted epitopes. Peptides were 14 to 19 amino acids in length with their N-terminals “capped” by N-acetylation or by the presence of N-pyroglutamate, and C-terminals were amidated. All glutamine residues in native gluten-derived sequences that were predicted to be susceptible to human transglutaminase-2 were replaced with glutamate residues.²⁶ Peptides were combined to yield a final lyophilized mixture of all 71 peptides. Unlike the smaller gluten peptide pools, the P71 was not immediately soluble in normal saline but did dissolve in sterile DMSO to 10 mg per peptide/mL before being diluted in phosphate buffered saline to desired concentrations. Amongst the 71 peptide sequences in P71, 14 were synthesized separately to greater than 95% purity and combined in three different mixtures: P3 (3 peptides, 15-16 amino acids), P14 (14 peptides, 14-19 amino acids) and P13 (13 peptides, 14-18 amino acids) (Table 6). An alternative equimolar solution of 13 peptides corresponding to Peptides 1, 2, and 3 and 10 of the peptides in P14 was prepared from individual peptide solutions initially in normal saline (P13alt).

Peripheral Blood Responses to Gluten Peptide Pools after Oral Gluten Challenge

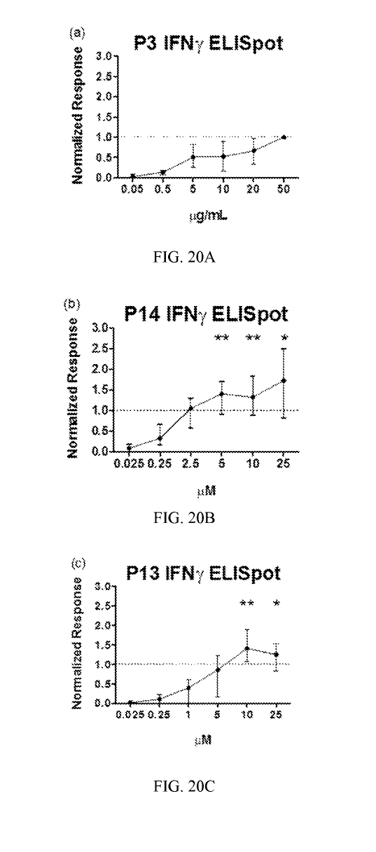

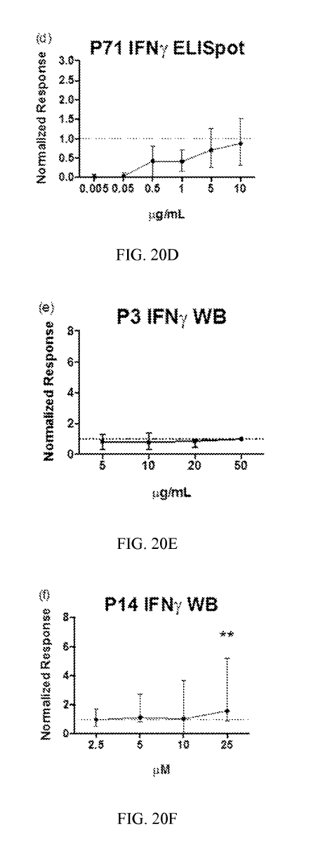

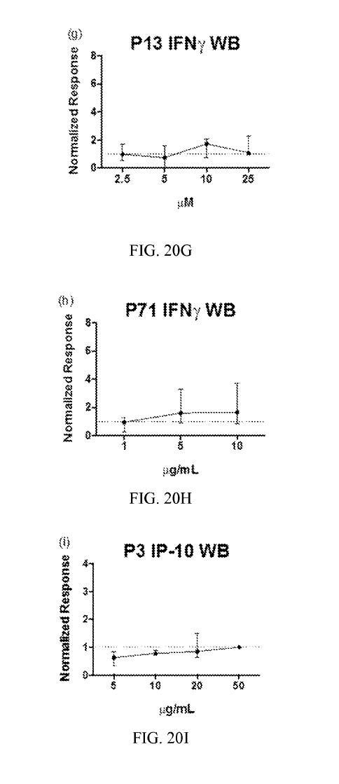

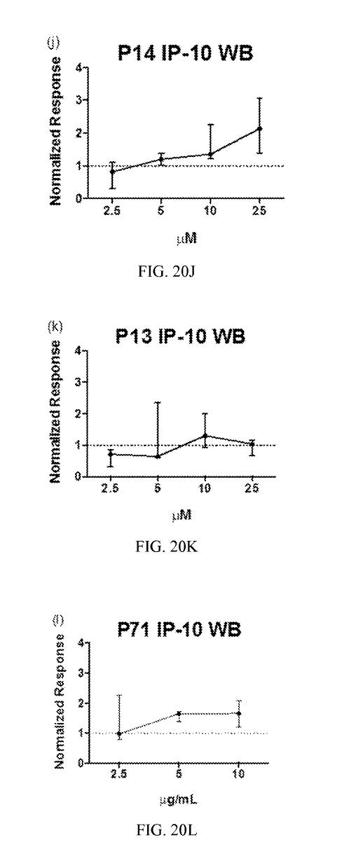

Whole blood IP-10 release stimulated by each of the four gluten peptide pools (P3, P13, P14, and P71) was increased in all ten subjects after oral gluten challenge (FIG. 17C, Table 7, FIGS. 20E-H) and consistently reached statistical significance. In contrast, the change in IFNγ responses to gluten peptide pools after oral challenge were not as pronounced or as consistent as IP-10 (FIGS. 17A and B). Four subjects failed to mount IFNγ ELISpot responses greater than 10 SFU/1.2 million PBMC or show an increased response after oral gluten challenge (subjects 1, 2, 5, and 7), and three showed no increase in IFNγ response to gluten peptide pools in 96-well whole blood assay formats (1, 2 and 5). Subjects 1 and 2 were also negative when IFNγ release was measured by ELISA in plasma from whole blood collected after gluten challenge that had been incubated with P3 in QuantiFERON® NIL tubes (Table 8). All subjects responded strongly to the recall MHC Class I epitope pool CEF in all four of IFNγ and IP-10 assay formats, but overall there was no statistically significant change after oral gluten challenge (FIG. 17).

TABLE 7
Responses to gluten peptide pools in whole blood cytokine release assays
Response relative to medium only - Stimulation Index (median, 95% confidence interval)

| Assay | Sample | N | P3 10 μg/mL | P14 5 μM | P13 5 μM | P71 10 μg/mL |
|---|---|---|---|---|---|---|
| IFNγ | Day-0 | 10 | 1.3 (1.1-2.1) | 1.3 (1.1-2.1) | 1.2 (1.0-2.0) | 1.5 (1.0-2.7) |
| IFNγ | Day-6 | 10 | 5.5 (0.8-49) | 7.7 (0.92-70) | 3.7 (0.9-43) | 13 (1.2-76) |
| IP-10 | Day-0 | 10 | 1.5 (1.2-2.0) | 3.1 (1.0-5.9) | 2.7 (1.1-4.8) | 4.6 (1.1-6.6) |
| IP-10 | Day-6 | 10 | 13 (3.8-24) | 14 (5.6-26) | 10.69 (6.5-17) | 17 (8.9-26) |

TABLE 8
Day-6 whole blood IFNγ release by ELISA

| | NIL | P3^{#} | P3- | P3 | P3 | CEF^{##} | CEF |
|---|---|---|---|---|---|---|---|
| Subject | pg/mL | pg/mL | NIL | SI | response* | pg/mL | SI |
| 1 | 2.0 | 5.7 | 3.8 | 2.9 | − | 297 | 152 |
| 2 | 9.6 | 12 | 1.4 | 1.1 | − | 654 | 68 |
| 3 | 6.5 | 130 | 124 | 20 | + | 198 | 30 |
| 4 | 2.0 | 100 | 98 | 51 | + | 226 | 116 |
| 5 | 4.6 | 17 | 12 | 3.6 | + | 925 | 203 |
| 6 | 2.6 | 30 | 28 | 12 | + | 1810 | 707 |
| 7 | 5.6 | 28 | 23 | 5.0 | + | 2000 | 357 |
| 8 | 2.2 | 48 | 45 | 21 | + | 93 | 42 |
| 9 | 28 | 102 | 74 | 3.6 | + | 1110 | 39 |
| 10 | 2.0 | 65 | 63 | 33 | + | 354 | 181 |

^{#}P3 50 μg/mL;
^{##}CEF 1 μg/mL;
*Positive response to P3 is defined as P3-NIL pg/mL >7.2 and P3/NIL >1.25

### Peripheral Blood Responses to Individual Gluten Peptides

The baseline frequency of memory T cells specific for gluten epitopes and the relative dose of epitopes presented after oral challenge would be expected to determine the number and relative frequencies of gluten epitope-specific T cells circulating after oral gluten challenge. Cytokine release responses to the 16 constituent peptides in P3, P14 and P13 were compared using blood collected after oral gluten challenge (Table 9 and 10).

TABLE 9
Peptide pool compositions and responses to individual gluten peptides

| | | | | IFNγ WB | IP-10 WB | |
|---|---|---|---|---|---|---|
| | | | | MAGPIX | MAGPIX | IFNγ ELISpot |
| | | | | % max | % max | % max |
| | | | HLA-DQ | Mean | Mean | Mean |
| P3 | P14 | P13 | restriction | (rank)/n = 8 | (rank)/n = 10 | (rank)/n = 5 |
| | Peptide | | DQ2.5 | 94 (1) | 82 (1) | 100 (1) |
| | 14 | | | | | |
| Peptide 3 | | Peptide 3 | DQ2.5 | 62 (2) | 65 (2) | 53 (2) |
| Peptide 1 | | Peptide 1 | DQ2.5 | 33 (3) | 58 (3) | 13 (10) |
| | Peptide | | DQ2.5 | 31 (4) | 42 (7) | 24 (5) |
| | 16 | | | | | |
| | Peptide 6 | Peptide 6 | DQ2.5/2.5 + 8/8 | 28 (5) | 43 (6) | 21 (8) |
| | Peptide | | DQ2.5 | 27 (6) | 49 (4) | 25 (4) |
| | 2* | | | | | |
| | Peptide | | DQ2.5 | 25 (7) | 33 (10) | 13 (11) |
| | 15 | | | | | |
| | Peptide 8 | Peptide 8 | DQ2.5 | 25 (8) | 42 (8) | 37 (3) |
| Peptide | | Peptide 2 | DQ2.5 | 23 (9) | 43 (5) | 19 (9) |
| 2* | | | | | | |
| | Peptide 4 | Peptide 4 | DQ2.5/2.5 + 8/8 | 15 (10) | 38 (9) | 24 (6) |
| | Peptide 5 | Peptide 5 | DQ2.5/2.5 + 8/8 | 11 (11) | 25 (11) | 22 (7) |
| | Peptide 7 | Peptide 7 | DQ2.5 | 9 (12) | 12 (12) | 5 (13) |
| | Peptide | Peptide | DQ2.2 | 7 (13) | 10 (13) | 2 (16) |
| | 13 | 13 | | | | |
| | Peptide | Peptide | DQ2.5/2.5 + 8/8 | 4 (14) | 1 (15) | 8 (12) |
| | 10 | 10 | | | | |
| | Peptide 9 | Peptide 9 | DQ8/2.5 + 8/8 | 2 (15) | 0 (17) | 1 (17) |
| | Peptide | Peptide | DQ2.5 | 2 (16) | 3 (14) | 3 (15) |
| | 12 | 12 | | | | |
| | Peptide | Peptide | DQ2.5/2.5 + 8/8/ | 0 (17) | 1 (16) | 3 (14) |
| | 11 | 11 | 2.2 + 8 | | | |

*Peptide 2 was re-synthesised by Pepscan for inclusion in P14 and assessed at a concentration of 10 μg/mL (5.45 μM). Peptide 2 prepared by CSBio was tested at 5 μM, and used to prepare P3 and P13 pools. The two versions of Peptide 2 were assessed separately. Individual peptides are those identified in Table 6.

TABLE 10
Percent of maximal response to individual gluten peptides by subjects mounting
elevated responses in cytokine assays

| | Subject | | | | | | | | | | |
|---|---|---|---|---|---|---|---|---|---|---|---|
| | 3 | 3 | 3 | 10 | 10 | 10 | 4 | 4 | 4 | 2 | 2 |
| | Cytokine | | | | | | | | | | |
| | IFNg | IP-10 | ELISp | IFNg | IP-10 | ELISp | IFNg | IP-10 | ELISp | IFNg | IP-10 |
| | Units | | | | | | | | | | |
| | pg/mL | pg/mL | SFU | pg/mL | pg/mL | SFU | pg/mL | pg/mL | SFU | pg/mL | pg/mL |
| Max. | 799 | 9492 | 18 | 229 | 8922 | 11 | 136 | 9463 | 36 | 116 | 3646 |
| Peptide | 100 | 100 | 100 | 100 | 100 | 100 | 100 | 100 | 100 | 53 | 31 |
| 14 (% of | | | | | | | | | | | |
| max) | | | | | | | | | | | |
| Peptide 3 | 92 | 100 | 50 | 81 | 100 | 9 | 66 | 100 | 83 | 35 | 14 |
| (% of | | | | | | | | | | | |
| max) | | | | | | | | | | | |
| Peptide 1 | 14 | 100 | 11 | 7 | 34 | 9 | 26 | 96 | 22 | 100 | 100 |
| (% of | | | | | | | | | | | |
| max) | | | | | | | | | | | |
| Peptide | 13 | 100 | 22 | 2 | 11 | 27 | 16 | 69 | 31 | 65 | 45 |
| 16 (% of | | | | | | | | | | | |
| max) | | | | | | | | | | | |
| Peptide 6 | 10 | 100 | 11 | 20 | 56 | 9 | 54 | 100 | 56 | 58 | 21 |
| (% of | | | | | | | | | | | |
| max) | | | | | | | | | | | |
| Peptide 2 | 17 | 100 | 0 | 26 | 87 | 18 | 51 | 100 | 89 | 33 | 33 |
| (% of | | | | | | | | | | | |
| max) | | | | | | | | | | | |
| Peptide | 5 | 100 | 11 | 7 | 17 | 0 | 24 | 67 | 17 | 46 | 30 |
| 15 (% of | | | | | | | | | | | |
| max) | | | | | | | | | | | |
| Peptide 8 | 24 | 100 | 44 | 45 | 94 | 27 | 39 | 100 | 64 | 35 | 5 |
| (% of | | | | | | | | | | | |
| max) | | | | | | | | | | | |
| Peptide 2 | 20 | 100 | 11 | 8 | 30 | 0 | 48 | 100 | 47 | 15 | 35 |
| (% of | | | | | | | | | | | |
| max) | | | | | | | | | | | |
| Peptide 4 | 13 | 100 | 17 | 11 | 30 | 27 | 24 | 100 | 44 | 33 | 19 |
| (% of | | | | | | | | | | | |
| max) | | | | | | | | | | | |
| Peptide 5 | 5 | 100 | 17 | 27 | 60 | 55 | 13 | 46 | 19 | 39 | 13 |
| (% of | | | | | | | | | | | |
| max) | | | | | | | | | | | |
| Peptide 7 | 2 | 60 | 0 | 1 | 3 | 9 | 8 | 20 | 11 | 60 | 7 |
| (% of | | | | | | | | | | | |
| max) | | | | | | | | | | | |
| Peptide | 3 | 70 | 6 | 0 | 0 | 0 | 0 | 0 | 6 | 47 | 16 |
| 13 (% of | | | | | | | | | | | |
| max) | | | | | | | | | | | |
| Peptide | 0 | 0 | 17 | 9 | 9 | 9 | 0 | 0 | 3 | 23 | 0 |
| 10 (% of | | | | | | | | | | | |
| max) | | | | | | | | | | | |
| Peptide 9 | 0 | 0 | 0 | 0 | 0 | 0 | 0 | 0 | 0 | 19 | 0 |
| (% of | | | | | | | | | | | |
| max) | | | | | | | | | | | |
| Peptide | 0 | 9 | 6 | 1 | 0 | 0 | 0 | 5 | 0 | 13 | 3 |
| 12 (% of | | | | | | | | | | | |
| max) | | | | | | | | | | | |
| Peptide | 0 | 6 | 0 | 0 | 0 | 0 | 0 | 2 | 3 | 0 | 0 |
| 11 (% of | | | | | | | | | | | |
| max) | | | | | | | | | | | |

Individual peptides are those identified in Table 6.

| | Subject | | | | | | | | | | | |
|---|---|---|---|---|---|---|---|---|---|---|---|---|
| | 6 | 6 | 6 | 9 | 9 | 8 | 8 | 8 | 7 | 7 | 5 | 1 |
| | Cytokine | | | | | | | | | | | |
| | IFNg | IP-10 | ELISp | IFNg | IP-10 | IFNg | IP-10 | ELISp | IFNg | IP-10 | IP-10 | IP-10 |
| | Units | | | | | | | | | | | |
| | pg/mL | pg/mL | SFU | pg/mL | pg/mL | pg/mL | pg/mL | SFU | pg/mL | pg/mL | pg/mL | pg/mL |
| Max. | 72 | 5863 | 16 | 67 | 8349 | 47 | 4019 | 17 | 17 | 2330 | 9336 | 2331 |
| Peptide | 100 | 100 | 100 | 98 | 100 | 100 | 91 | 100 | 99 | 100 | 100 | 2 |
| 14 (% of | | | | | | | | | | | | |
| max) | | | | | | | | | | | | |
| Peptide | 63 | 43 | 69 | 60 | 85 | 89 | 100 | 53 | 6 | 5 | 53 | 51 |
| 3 (% of | | | | | | | | | | | | |
| max) | | | | | | | | | | | | |
| Peptide | 25 | 23 | 25 | 43 | 75 | 6 | 3 | 0 | 43 | 39 | 14 | 100 |
| 1 (% of | | | | | | | | | | | | |
| max) | | | | | | | | | | | | |
| Peptide | 10 | 4 | 19 | 39 | 60 | 0 | 1 | 24 | 100 | 800 | 35 | 16 |
| 16 (% of | | | | | | | | | | | | |
| max) | | | | | | | | | | | | |
| Peptide | 4 | 0 | 31 | 69 | 100 | 6 | 8 | 0 | 2 | 1 | 8 | 34 |
| 6 (% of | | | | | | | | | | | | |
| max) | | | | | | | | | | | | |
| Peptide | 18 | 30 | 0 | 33 | 70 | 39 | 32 | 18 | 3 | 3 | 31 | 0 |
| 2 (% of | | | | | | | | | | | | |
| max) | | | | | | | | | | | | |
| Peptide | 5 | 0 | 31 | 100 | 69 | 4 | 5 | 6 | 6 | 6 | 33 | 4 |
| 15 (% of | | | | | | | | | | | | |
| max) | | | | | | | | | | | | |
| Peptide | 6 | 2 | 31 | 5 | 31 | 42 | 39 | 18 | 0 | 0 | 45 | 0 |
| 8 (% of | | | | | | | | | | | | |
| max) | | | | | | | | | | | | |
| Peptide | 3 | 3 | 13 | 72 | 100 | 2 | 6 | 24 | 14 | 11 | 16 | 29 |
| 2 (% of | | | | | | | | | | | | |
| max) | | | | | | | | | | | | |
| Peptide | 1 | 2 | 19 | 29 | 48 | 9 | 7 | 12 | 1 | 3 | 51 | 18 |
| 4 (% of | | | | | | | | | | | | |
| max) | | | | | | | | | | | | |
| Peptide | 1 | 1 | 0 | 0 | 10 | 0 | 4 | 18 | 6 | 2 | 13 | 0 |
| 5 (% of | | | | | | | | | | | | |
| max) | | | | | | | | | | | | |
| Peptide | 0 | 0 | 6 | 0 | 17 | 0 | 3 | 0 | 0 | 0 | 12 | 0 |
| 7 (% of | | | | | | | | | | | | |
| max) | | | | | | | | | | | | |
| Peptide | 5 | 5 | 0 | 0 | 0 | 0 | 0 | 0 | 1 | 0 | 6 | 0 |
| 13 (% of | | | | | | | | | | | | |
| max) | | | | | | | | | | | | |
| Peptide | 0 | 0 | 6 | 0 | 17 | 0 | 3 | 0 | 0 | 0 | 12 | 0 |
| 10 (% of | | | | | | | | | | | | |
| max) | | | | | | | | | | | | |
| Peptide | 0 | 0 | 0 | 0 | 0 | 0 | 0 | 6 | 0 | 0 | 0 | 0 |
| 9 (% of | | | | | | | | | | | | |
| max) | | | | | | | | | | | | |
| Peptide | 0 | 0 | * | 0 | 1 | 0 | 0 | 6 | 0 | 0 | 4 | 11 |
| 12 (% of | | | | | | | | | | | | |
| max) | | | | | | | | | | | | |
| Peptide | 0 | 0 | 6 | 0 | 0 | 0 | 0 | 6 | 0 | 0 | 0 | 0 |
| 11 (% of | | | | | | | | | | | | |
| max) | | | | | | | | | | | | |

* indicates that the ELISpot response to Peptide 12 was negative in one of two duplicate ELISpot assays in this subject

The most active peptide tested was a barley-derived 19mer, Peptide 14, which corresponds to the partially deamidated sequence of B1 hordein (Genbank CAA60681.1) residues 21 to 37 with the addition of pyroGlu-Pro (ZP) at the N-terminal and an amide group at the C-terminal (*): ZPEQPFPEQPIPEQPQPYP* (SEQ ID NO: 283). Peptide 3 was consistently the second most active peptide after Peptide 14. IFNγ ELISpot responses to Peptide 3 10 μg/mL (5.3 μM) were only one-half of those to Peptide 14 5 μM. In whole blood incubations with Peptide 3, IP-10 release was one-quarter less and IFNγ release one-third less than that stimulated by Peptide 14. Peptide 14 is closely related to Peptide 3. Peptide 14 differs from Peptide 3 by the insertion of EQPFP (SEQ ID NO: 284) following N-pyroGlu-Pro and by removal of the C-terminal di-glutamine. The most likely explanation for Peptide 14's greater activity is that it comprises two additional epitopes (EQPFPEQPI (SEQ ID NO: 20), and PFPEQPIPE (SEQ ID NO: 21)) further to those in Peptide 3 (EQPIPEQPQ (SEQ ID NO: 5) and PIPEQPQPY (SEQ ID NO: 6)). Indeed, deamidation of the peptide named B16 QQQPFPQQPIPQ (SEQ ID NO: 285) by transglutaminase was confirmed to generate an immunogenic sequence recognized by circulating T cells after oral challenge of HLA-DQ2.5 CD subjects with pure barley.³ The sequences EQPFPEQPI (SEQ ID NO: 20) and PFPEQPIPE (SEQ ID NO: 21) were also predicted to be candidate HLA-DQ2.5-restricted epitopes.²⁷

Half the daily prolamin dose in the oral gluten challenge was wheat gluten (approximately half as gliadins and half as glutenins), one third was from barley and one sixth was from rye. Despite the substantial load of wheat gluten, peptides (Peptide 1, 15 and 16) comprising overlapping epitopes dominant after oral wheat challenge (DQ2.5-glia-α1a, DQ2.5-glia-α1b and DQ2.5-glia-α2) were no more than two-thirds as active as Peptide 14. Peptide 1, which includes DQ2.5-glia-α1a and DQ2.5-glia-α2 epitopes, was the most active of this group of related peptides.

Peptides 2, 6 and 8 were each approximately one-half to one-fifth as active as Peptide 14 in the three cytokine release assays. These peptides comprised sequences known to be HLA-DQ2.5-restricted epitopes. Peptide 6 also encompassed sequences recognized by T cells in blood after oral challenge in HLA-DQ8+ celiac disease subjects. Peptides 4 and 5 were from one-half to one-eighth as active as Peptide 14. They had been included in the larger pools because of their contribution to the T cell response after oral gluten challenge in patients with celiac disease who are HLA-DQ2.5+ and/or HLA-DQ8+. Peptides 7 and 12 stimulated cytokine release that was on average no more than 10% of that to Peptide 14. This finding was at odds with previous findings following oral challenges with pure wheat or rye.³

Cytokine release stimulated by peptides comprising important HLA-DQ8- or HLA-DQ2.2-restricted epitopes (Peptides 9, 10, 11, and 13) was weak or not distinguishable from medium alone. However, HLA-DQ8+ and DQ2.2+ subjects who do not also carry HLA-DQ2.5 will be required to fully assess the immunogenicity of these peptides.

In summary, the relative magnitude and rank order of responses to peptides in HLA-DQ2.5+ subjects was generally consistent across each of the cytokine release assays. With the exceptions of Peptides 7 and 12, peptides that had been selected for their importance in HLA-DQ2.5+ CD were at least as active as Peptide 2. The most active peptide was Peptide 14, a hordein-derived 19mer that included up to four overlapping epitopes. Inclusion of Peptide 14 in P14, and its replacement by Peptide 3 in P13, is likely to account for P14 but not P13 being significantly more bioactive than P3.

Building upon these findings a further 13-peptide pool, P13alt, was designed to retain the higher T-cell stimulatory activity of P14, but reduce the number and length of constituent peptides as well as include the three peptides in P3. In the 13-peptide pool named P13alt, the highly immunogenic 19mer, Peptide 14 sequence is divided up between the 16mers Peptide 3 and Peptide 17 which both include overlapping 9mer cores predicted to be immunogenic in Peptide 14. In P13alt, only the more active peptide with the α-gliadin-derived epitopes DQ2.5-glia-α1 and DQ2.5-glia-α2 is included (Peptide 1) while Peptide 16 has been omitted. Peptide 8, which was included in P14, is replaced in P13alt by a closely related sequence frame-shifted by one amino-acid to ensure that the two overlapping core 9mers are flanked at both the N- and C-terminals by at least 2 amino-acids. Peptides 7 and 12 are omitted from P13alt because even though they included core sequences predicted to be HLA-DQ2.5-restricted epitopes their immunogenicity in blood collected from HLA-DQ2.5+ celiac disease subjects after gluten challenge was weak or absent. Peptides 2,4-7, 9-11, 13, and 15 present in P14 are also included in P13alt.

### Optimizing Gluten Peptide Pools for Maximal Cytokine Release

Before gluten challenge, whole blood IFNγ release was similar for P3 and the other gluten peptide pools, but IP-10 release stimulated by P3 10 μg/mL was only half that for P14 5 μM and a third that of P71 10 μg/mL (Table 11). Oral gluten challenge was followed by significant increases in cytokine release; in six subjects IP-10 levels in plasma after whole blood incubation with gluten peptides were at or above the maximal quantifiable concentration. However, after normalizing each subject's cytokine release assay responses against their own response to the highest tested concentration of P3 (50 μg/mL), median responses to the highest tested concentration of P14 (25 μM) were 60% greater than P3 50 μg/mL in both the IFNγ ELISpot and whole blood IFNγ release assays (p<0.01, and p<0.05, respectively) (Table 11 and FIG. 18). Statistical significance was not formally tested in the whole blood IP-10 release assay because six subjects mounted supra-maximal responses, but in the four subjects whose levels were within the dynamic range of the assay, responses to P14 (251.1M) were a median of 2.1× (range: 1.4-3.1) greater than to P3 50 μg/mL. The increase in responses to P13 and P71 compared to P3 were not as pronounced as for P14, but did reach statistical significance for P13 (25 μM) in the IFNγ ELISpot. Responses to P71 (10 μg/mL) were 1.7× higher than P3 (50 μg/mL) in the whole blood IFNγ and IP-10 release assays, but neither was statistically significant. To test whether normalization of data against P3 50 μ/mL was valid, data were also normalized against P3 20μ/mL. According to this analysis whole blood IFNγ release stimulated by P14 25 uM was 70% greater than P3 50 ug/mL (p<0.014, two-tail Wilcoxon matched-pairs signed rank test). Furthermore, whole blood IFNγ release stimulated by P14 10 μM was greater than P3 20 μg/mL (p<0.04), and P14 5 μM was greater than P3 10 μg/mL (p<0.006). In conclusion, responses to P14 in cytokine release assays were significantly greater than P3.

TABLE 11
Responses to gluten peptide pools in cytokine release assays
Response on Day-6 relative to P3 (median, range)

| Cytokine | Assay | P3 50 μg/mL | P14 25 μM | P13 25 μM | P71 10 μg/mL |
|---|---|---|---|---|---|
| IFNγ | ELISpot 6¹ | 1 | 1.6 (0.82-2.3) | 1.3 (0.82-1.5) | 0.91 (0.31-1.5) |
| | | | p < 0.05 | p < 0.05 | |
| IFNγ | WB 7² | 1 | 1.6 (0.9-5.2) | 1.1 (0.95-2.3) | 1.7 (0.84-3.7) |
| | | | p < 0.01 | | |
| IP-10 | WB 4³ | 1 | 2.1 (1.4-3.1) | 1.0 (0.68-1.2) | 1.7 (1.2-2.1) |

¹Data from subjects with a P3 (50 μg/mL)-specific response <3x above medium alone or <10 SFU (sum of three wells with 0.4 million PBMC/well) were excluded from analysis.
²Data from subjects with a P3 (50 μg/mL)-specific response <1.5x medium alone were excluded from analysis.
³All subjects showed a P3 (50 μg/mL)-specific response <1.5x medium alone, but 6 were excluded from analysis because responses were above 10,000 pg/mL, the maximal level of quantitation.

### Comparison of Whole Blood IFNγ and IP-10 Release Measured by Bead Assays

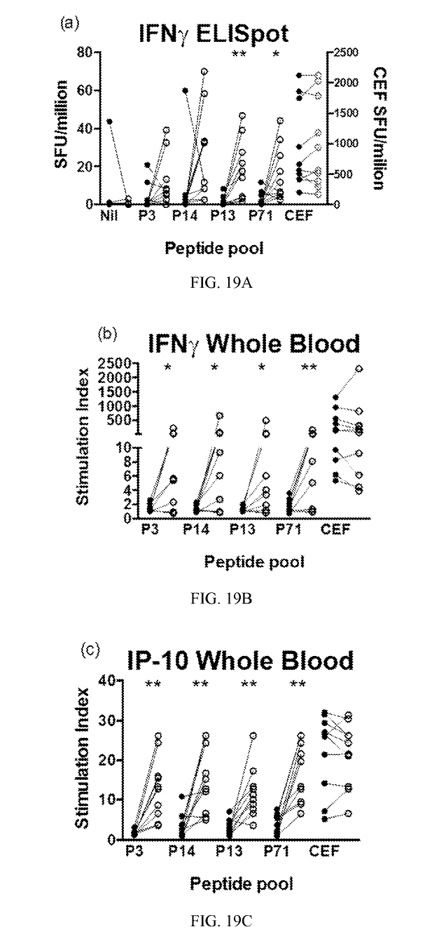

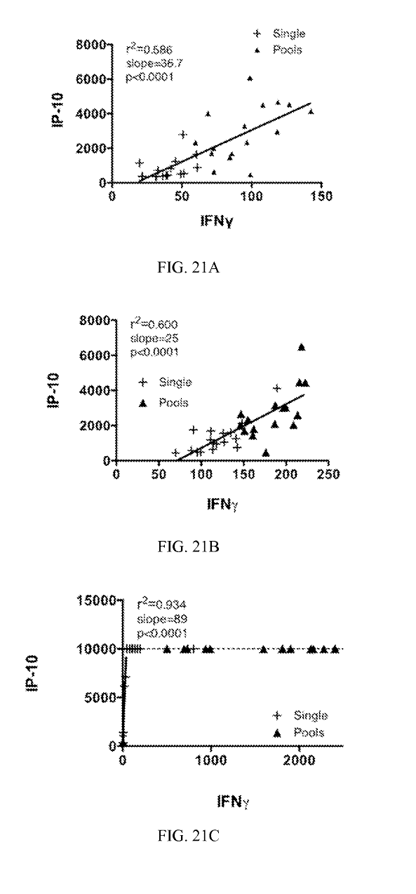

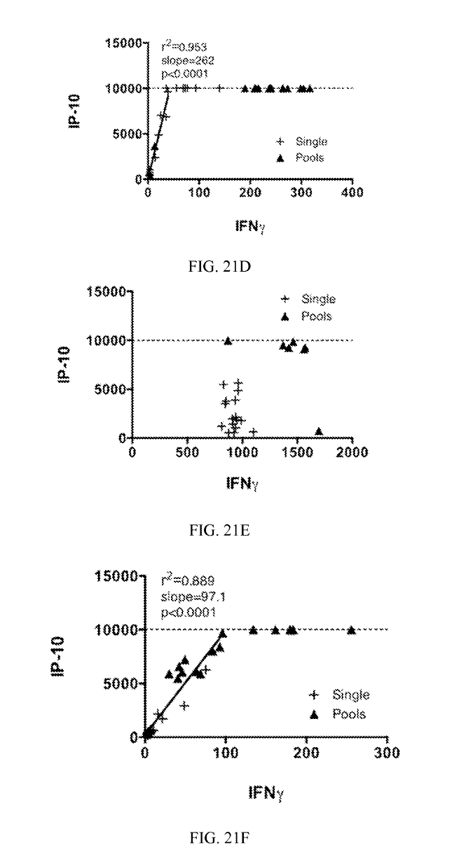

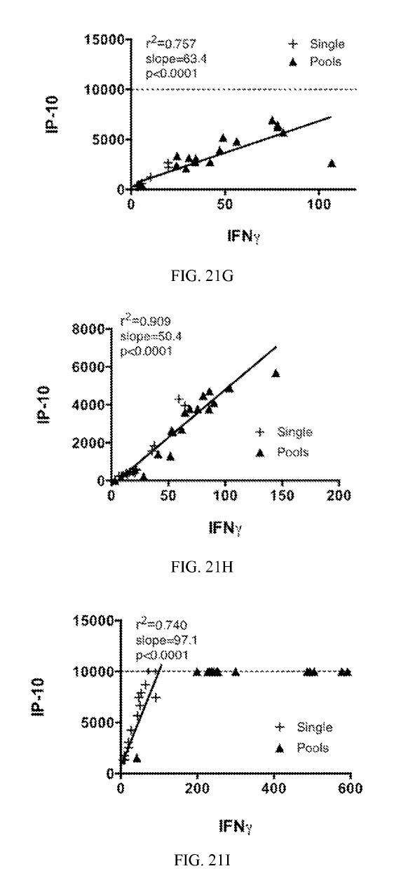

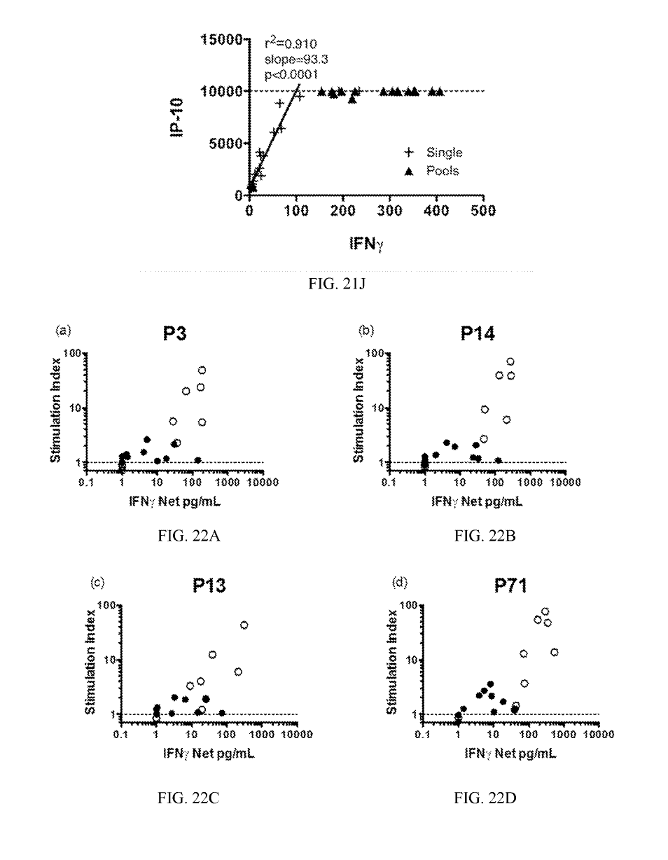

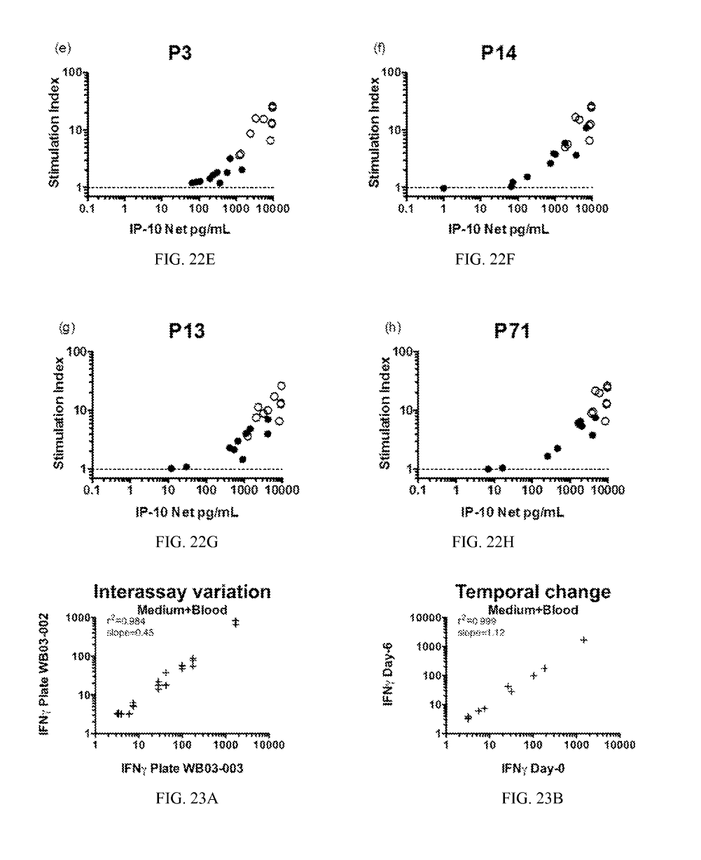

Within the dynamic range of the cytokine bead assay, there was a close correlation between individual subject's whole blood IP-10 and IFNγ release stimulated by pools or single gluten peptides (FIG. 19). Furthermore, IP-10 and IFNγ levels in plasma samples from blood collected from the same subject on Day-0 or Day-6 that were incubated with medium alone and later measured in the same or separate assays were also closely correlated (FIG. 21). However, the linear relationship between IP-10 and IFNγ was not found when data from the cohort of all ten subjects was pooled (FIG. 22), and in one subject (Subject 5) with substantially higher measured levels of plasma IFNγ in blood incubated with medium alone there was no significant correlation.

These findings were consistent with IP-10 secretion being in direct proportion to IFNγ during whole blood incubation. To test whether whole blood release of IP-10 may be more sensitive than IFNγ for detection of rare gluten-specific T cells, IP-10 levels corresponding to the threshold for “positive” whole blood IFNγ release measured by ELISA were applied to data for IFNγ and IP-10 release measured by bead assay (Table 12).

TABLE 12
IFNγ secretion required for “positive” IFNγ or IP-10 response¹

| | Subject | | | | | | | | | | Median |
|---|---|---|---|---|---|---|---|---|---|---|---|
| | 1 | 2 | 3 | 4 | 5 | 6 | 7 | 8 | 9 | 10 | (range) |
| IFNγ Day-6 medium | 76 | 125 | 3 | 4 | 1234 | 3 | 5 | 23 | 33 | 6 | 15 |
| only | | | | | | | | | | | (3-1234) |
| IFNγ Day-6 medium | 84 | 132 | 10 | 11 | 1241 | 11 | 12 | 30 | 40 | 14 | 22 |
| only +7.2 (A) | | | | | | | | | | | (10-1241) |
| IFNγ Day-6 medium | 95 | 156 | 4 | 4 | 1542 | 4 | 6 | 29 | 42 | 8 | 18 |
| only * 1.25 (B) | | | | | | | | | | | (4-1542) |
| IFNγ “positive” | 95 | 156 | 10 | 11 | 1542 | 11 | 12 | 30 | 42 | 14 | 22 |
| threshold (the | | | | | | | | | | | (10-1542) |
| greater A or B) | | | | | | | | | | | |
| IP-10 Day-6 medium | 458 | 476 | 445 | 474 | 709 | 385 | 331 | 256 | 1591 | 932 | 466 |
| only | | | | | | | | | | | (256-1591) |
| IP-10 Day-6 * 1.25 = | 572 | 595 | 556 | 592 | 886 | 481 | 414 | 320 | 1988 | 1165 | 582 |
| “positive” threshold | | | | | | | | | | | (320-1988) |
| Slope IP-10/IFNg | 37 | 25 | 231 | 252 | ND | 97 | 63 | 50 | 97 | 93 | 93 |
| Day-6 | | | | | | | | | | | (25-252) |
| Net increase in IP-10 | 114 | 119 | 111 | 118 | 177 | 96 | 83 | 64 | 398 | 233 | 116 |
| at IP-10 positive | | | | | | | | | | | (64-398) |
| “threshold”² | | | | | | | | | | | |

Elevation in IFNγ to reach threshold:

| | | | | | | | | | | | |
|---|---|---|---|---|---|---|---|---|---|---|---|
| For positive IP-10 | 3.1 | 4.8 | 0.5 | 0.5 | NA | 1.0 | 1.3 | 1.3 | 4.1 | 2.5 | 1.3 |
| | | | | | | | | | | | (0.5-4.8) |
| For positive IFNγ | 19 | 31 | 7.2 | 7.2 | 308 | 7.2 | 7.2 | 7.2 | 8.3 | 7.2 | 7.2 |
| | | | | | | | | | | | (7.2-308) |

¹Assay positive threshold for IFNγ was based on that for ELISA: net elevation of 7.2 pg/mL above medium alone (NIL), and stimulation index (measured IFNγ pg/mL/NIL) >1.25.
²Elevation in IP-10 stimulated by secretion of IFNγ was determined according to the slope of the regression line comparing IP-10 and IFNγ (FIG. 20)

Plasma IFNγ concentration preferably should be at least 7.2 pg/mL greater than blood incubated with medium only (NIL), and the concentration ratio to NIL preferably should be greater than 1.25 for a “positive” whole blood IFNγ ELISA. Applying these criteria to IFNγ release measured by bead assay on Day-6, the threshold concentration for positive IFNγ responses was between 10 and 42 pg/mL in five subjects, but substantially higher (95, 156 and 1542 pg/mL) in three subjects (1, 2 and 5) with elevated responses to medium only. Indeed, these three subjects were regarded as having negative IFNγ responses to P3 on both Day-0 and Day-6, in contrast to the other seven who were regarded as being “positive” on Day-6.

The slope of the regression line linking bead assay IP-10 to IFNγ levels on Day-6 for nine subjects indicated that for every pg/mL elevation of IFNγ the concentration of IP-10 increased by a median of 96 pg/mL (range: 78-479). Median NIL levels of IP-10 were 466 pg/mL (range: 256-1591). If a positive response for IP-10 was regarded as being 25% above the response to NIL, then a median elevation in IP-10 concentration of at least 116 pg/mL (range: 64-398) should indicate a “positive” response. The median devotion in IFNγ levels above NIL that would be predicted to translate to elevating IP-10 levels to the threshold for “positive” is therefore 1.3 pg/mL (range: 0.5-48), corresponding to a median of 6.1 (range: 2.0-15.3) times lower than that required for a positive IFNγ response. This outcome is consistent with only one subject being positive for IFNγ release to P3 before oral gluten challenge (median P3-NIL: 4.625, range: −0.1-143; P3/NIL: median 1.27, range: 1.0-2.6) compared to 7 of 10 subjects having IP-10 stimulation indices >1.25 for P3 10 pg/mL on Day-(P3-NIL median: 275 pg/mL, range: 65-1453; P3/NIL: 1.5, 1.2-3.2). Interestingly, P14 elicited responses that were more pronounced than those to P3 on Day-0 (IP-10 P14-NIL median: 839 pg/mL, range: −13-6936; P14/N1L: 3.1, 1.0-10.8), but still only 7 of 10 subjects had IP-10 stimulation indices >1.25.

### CONCLUSIONS

In summary, whole blood IP-10 release is tightly correlated to IFNγ release stimulated by gluten peptides in celiac disease. According to the regression line linking IFNγ and IP-10 release, measuring plasma levels of IP-10 in whole blood incubated with gluten peptides is capable of detecting rare gluten-reactive T cells that are not detected by measuring IFNγ. Although the three peptides in P3 are confirmed to be potent stimuli for circulating T cells in celiac disease, expanding the diversity of epitopes in enlarged peptide pools can further enhance ex vivo detection and therapeutic targeting of gluten-reactive T cells. The findings that IP-10 but not IFNγ responses to P14 were more pronounced than those to P3 on Day-0 suggests that measuring whole blood IP-10 release stimulated by peptide pools based on P3 but expanded by additional peptides may overcome the need for oral gluten challenge to detect circulating gluten-reactive T cells in many patients with celiac disease. An assay such as this could provide an attractive diagnostic test for celiac disease.

### REFERENCES

1. Anderson, R. P. & Jabri, B. Vaccine against autoimmune disease: antigen-specific immunotherapy. Current opinion in immunology 25, 410-417 (2013).
2. Anderson, R. P., Degano, P., Godkin, A. J., Jewell, D. P. & Hill, A. V. In vivo antigen challenge in celiac disease identifies a single transglutaminase-modified peptide as the dominant A-gliadin T-cell epitope. Nature medicine 6, 337-342 (2000).
3. Tye-Din, J. A., et al. Comprehensive, quantitative mapping of T cell epitopes in gluten in celiac disease. Science translational medicine 2, 41ra51 (2010).
4. Koskinen, L., et al. Cost-effective HLA typing with tagging SNPs predicts celiac disease risk haplotypes in the Finnish, Hungarian, and Italian populations. Immunogenetics 61, 247-256 (2009).
5. Anderson, R. P., et al. A novel serogenetic approach determines the community prevalence of celiac disease and informs improved diagnostic pathways. BMC medicine 11, 188 (2013).
6. Murray, J. A., et al. HLA DQ gene dosage and risk and severity of celiac disease. Clinical gastroenterology and hepatology: the official clinical practice journal of the American Gastroenterological Association 5, 1406-1412 (2007).
7. Karell, K., et al. HLA types in celiac disease patients not carrying the DQA1*05-DQB1*02 (DQ2) heterodimer: results from the European Genetics Cluster on Celiac Disease. Human immunology 64, 469-477 (2003).
8. Henderson, K. N., et al. A structural and immunological basis for the role of human leukocyte antigen DQ8 in celiac disease. Immunity 27, 23-34 (2007).
9. van de Wal, Y., et al. Selective deamidation by tissue transglutaminase strongly enhances gliadin-specific T cell reactivity. Journal of immunology 161, 1585-1588 (1998).
10. van de Wal, Y., et al. Glutenin is involved in the gluten-driven mucosal T cell response. European journal of immunology 29, 3133-3139 (1999).
11. Tollefsen, S., et al. HLA-DQ2 and -DQ8 signatures of gluten T cell epitopes in celiac disease. The Journal of clinical investigation 116, 2226-2236 (2006).
12. Kooy-Winkelaar, Y., et al. Gluten-specific T cells cross-react between HLA-DQ8 and the HLA-DQ2alpha/DQ8beta transdimer. Journal of immunology 187, 5123-5129 (2011).
13. Bodd, M., Kim, C. Y., Lundin, K. E. & Sollid, L. M. T-cell response to gluten in patients with HLA-DQ2.2 reveals requirement of peptide-MHC stability in celiac disease. Gastroenterology 142, 552-561 (2012).
14. Vader, W., et al. The gluten response in children with celiac disease is directed toward multiple gliadin and glutenin peptides. Gastroenterology 122, 1729-1737 (2002).
15. Ontiveros, N., Tye-Din, J. A., Hardy, M. Y. & Anderson, R. P. Ex-vivo whole blood secretion of interferon (IFN)-gamma and IFN-gamma-inducible protein-10 measured by enzyme-linked immunosorbent assay are as sensitive as IFN-gamma enzyme-linked immunospot for the detection of gluten-reactive T cells in human leucocyte antigen (HLA)-DQ2.5(+)-associated coeliac disease. Clinical and experimental immunology 175, 305-315 (2014).
16. Christophersen, A., et al. Tetramer-visualized gluten-specific CD4+ T cells in blood as a potential diagnostic marker for coeliac disease without oral gluten challenge. United European gastroenterology journal 2, 268-278 (2014).
17. Luster, A. D. & Ravetch, J. V. Biochemical characterization of a gamma interferon-inducible cytokine (IP-10). The Journal of experimental medicine 166, 1084-1097 (1987).
18. Cassatella, M. A., et al. Regulated production of the interferon-gamma-inducible protein-10 (IP-10) chemokine by human neutrophils. European journal of immunology 27, 111-115 (1997).
19. Dufour, J. H., et al. IFN-gamma-inducible protein 10 (IP-10; CXCL10)-deficient mice reveal a role for IP-10 in effector T cell generation and trafficking. Journal of immunology 168, 3195-3204 (2002).
20. Ruhwald, M., et al. CXCL10/IP-10 release is induced by incubation of whole blood from tuberculosis patients with ESAT-6, CFP10 and TB7.7. Microbes and infection/Institut Pasteur 9, 806-812 (2007).
21. Wang, S., et al. Evaluation of the diagnostic potential of IP-10 and IL-2 as biomarkers for the diagnosis of active and latent tuberculosis in a BCG-vaccinated population. PloS one 7, e51338 (2012).
22. National Institutes of Health Consensus Development Conference Statement on Celiac Disease, Jun. 28-30, 2004. Gastroenterology 128, S1-9 (2005).
23. Junker, Y., et al. Wheat amylase trypsin inhibitors drive intestinal inflammation via activation of toll-like receptor 4. The Journal of experimental medicine 209, 2395-2408 (2012).
24. Kern, F., et al. Analysis of CD8 T cell reactivity to cytomegalovirus using protein-spanning pools of overlapping pentadecapeptides. European journal of immunology 30, 1676-1682 (2000).
25. Maecker, H. T., et al. Use of overlapping peptide mixtures as antigens for cytokine flow cytometry. Journal of immunological methods 255, 27-40 (2001).
26. Vader, L. W., et al. Specificity of tissue transglutaminase explains cereal toxicity in celiac disease. The Journal of experimental medicine 195, 643-649 (2002).
27. Shan, L., et al. Identification and analysis of multivalent proteolytically resistant peptides from gluten: implications for celiac sprue. Journal of proteome research 4, 1732-1741 (2005).

### Example 4

### Assessment of Circulating Cytokine and Chemokine Levels in Connection with Administration with a Gluten Peptide Composition

### Objectives:

Assess a tolerable but active dose of the gluten peptide composition that elevates whole blood IP-10 release in patients with celiac disease following strict gluten-free diet (GFD).

### Patient Population:

Male or female patients aged 18 to 70 years with celiac disease on GFD.

### Key Inclusion Criteria:

Patient is between 18 and 70 years old (inclusive). Patient has been diagnosed with celiac disease on the basis of intestinal histology showing villous atrophy according to expert guidelines current at the time of diagnosis, e.g. ESPGHAN 1990(1990). GFD for at least one-month. Patients is positive for HLA-DQA1*05 and/or HLA-DQB1*02 and/or HLA-DQB1*0302 alleles, and if enrolled in Cohorts A-E is positive for both HLA-DQA1*05 and HLA-DQB1*02 (“HLA-DQ2.5+”). Patient is sero-negative for tTG IgA and is not IgA deficient if enrolled in Cohorts A-E.

### Study Design:

At the Screening visit the following will be performed: medical history, physical exam, vital signs (HR, SBP, DBP, RR, SaO₂, aural temperature) and ECG. Screening laboratory tests will include: HLA-DQA and HLA-DQB alleles, recombinant human tTG IgA, total IgA, full blood count with differential and platelet count, clotting, liver and renal function with urinalysis. Subjects will have completed a Celiac Dietary Adherence Test (CDAT) at or within a week of Screening visit. Subjects in Cohort F will also undergo an upper gastrointestinal ndoscopy (EGD) with distal duodenal (D2-3) biopsies for histology to determine D2-3 VH:CrD and TEL density. Treatment Day will be within 6 weeks of the Screening visit. Subjects in Cohorts A-D (n=8 per cohort) and Cohort E (n=16) will be sero-negative for tTG IgA and not IgA deficient, and all will be HLA-DQ2.5+. Each of Cohorts A-D will include two subjects who have no other HLA-DQA allele apart from HLA-DQA1*05 and no other HLA-DQB allele apart from HLA-DQB1*02 (homozygous for HLA-DQ2.5). Cohort E will include four subjects who are homozygous for HLA-DQ2.5. Cohorts A-E will enroll in parallel and assess a single intradermal administration of four dose levels of a gluten peptide composition or placebo (Cohort A: 30 μg, Cohort B: 45 μg, Cohort C: 60 μg, and Cohort D: 150 μg; and Cohort E: placebo) injected in 0.1 mL using a syringe fitted with a West Intradermal Adaptor. A planned interim analysis of data from Cohorts A-E will be performed once these cohorts are completed in order to select one dose level to be compared with placebo in Cohort F (n=30) and Cohort G (n=60). Subjects in Cohorts F and G will enroll in parallel, and within each cohort subjects will be randomized to receive a single intradermal dose of the gluten peptide composition or placebo in a 2:1 ratio. In Cohorts F and G, celiac disease patients will not be required to be sero-negative for tTG IgA, and will possess any of the HLA-DQA and HLA-DQB alleles associated with celiac disease (HLA-DQA1*05, HLA-DQB1*02, and HLA-DQB1*0302) not just HLA-DQ2.5. In all cohorts, planned assessments will be performed pre-dose, 2 h, 4 h, 6 h, 24 h and 6-days after dosing. The schedule of assessments and procedures in Cohorts F and G will be identical to one another except that EGD with D2-3 biopsy will be performed during Screening (before dosing) in Cohort F. Immediately pre-dose and, in some cases, repeatedly during follow-up, laboratory tests will include liver function, full blood examination with differential and platelet count, tTG-IgA, DGP-IgA, DGP-IgG, serum cytokines, whole blood RNA expression profiling, CRP, and gluten peptide-stimulated whole blood IP-10 release. In addition to standard adverse event monitoring, investigators will be required to grade severity of symptoms described in the FDA Guidance for Industry, “Toxicity Grading Scale for Healthy Adult and Adolescent Volunteers Enrolled in Preventive Vaccine Clinical Trials”, and also according to MedDRA v.15 Code 10052015 (“cytokine release syndrome”) at 2 h, 4 h, 6 h, 24 h and 6-days after dosing. Subjects will also complete a symptom diary before, and when cytokine levels are assessed. The GSRS will be completed pre-dose and on day-6 post-dose relating to symptoms experienced during the prior one week.

### Pharmacodynamic Assessments:

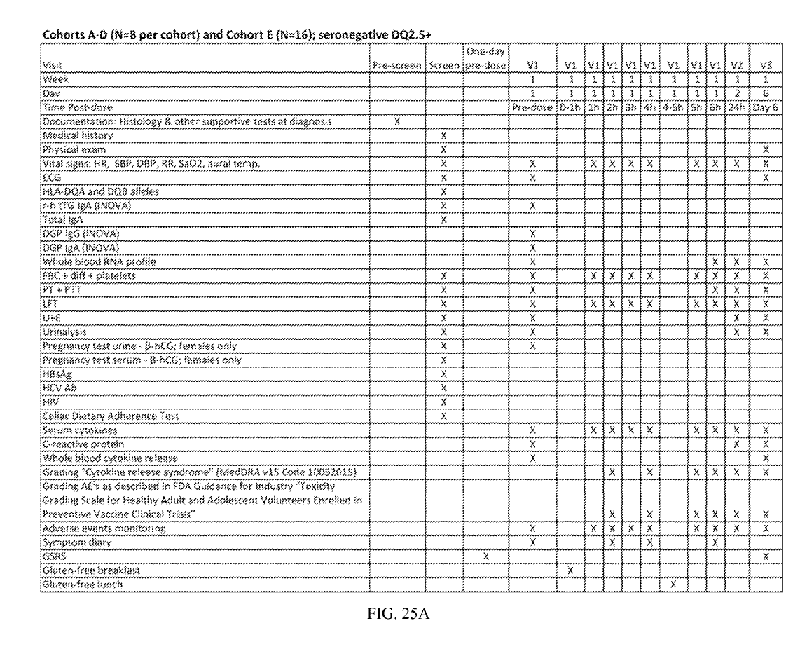

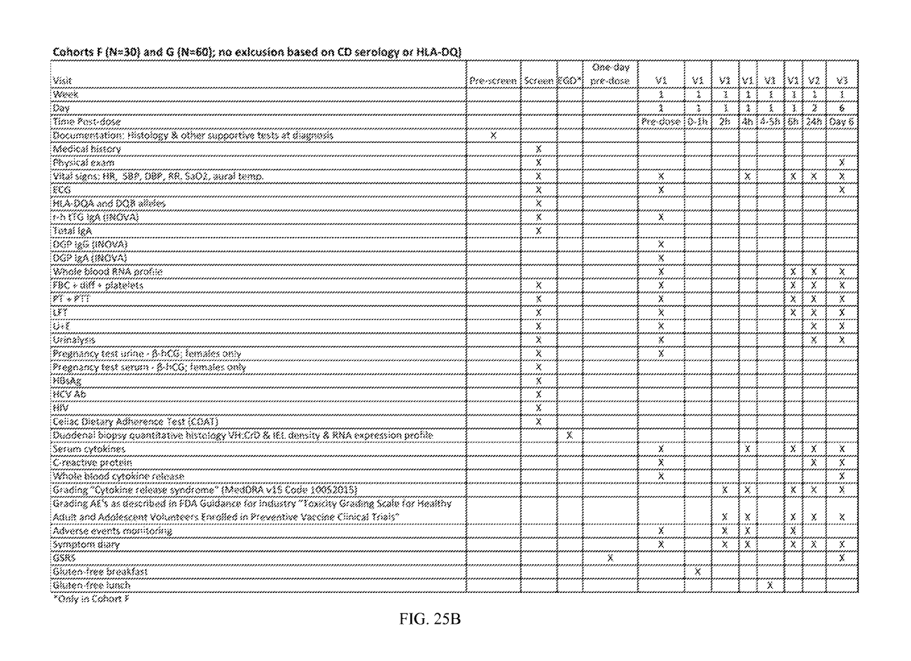

Gluten-peptide-stimulated whole blood IP-10 release will be assessed at the time points outlined in the Schedule of Assessments (SOA, see FIGS. 25A and 25B). Other measurements include serum cytokines, whole blood RNA expression profiling, and CRP.

### Study Drug, Dosage, and Route of Administration:

The gluten peptide composition administered includes 3 peptides in equimolar amounts in sodium chloride 0.9% USP: ELQPFPQPELPYPQPQ (SEQ ID NO: 232), EQPFPQPEQPFPWQP (SEQ ID NO: 233), and EPEQPIPEQPQPYPQQ (SEQ ID NO: 234). For each peptide in the composition, the N-terminal glutamate is a pyroglutamate and the carboxyl group of the C-terminal proline or glutamine is amidated.

Dosage regimens planned:

Cohort A: 30 μg n=8

Cohort B: 45 μg n=8

Cohort C: 60 μg n=8

Cohort D: 150 μg n=8

Cohort E: Placebo n=16

Cohort F: dose selected from 30, 45, 60 or 150 μg N=30

Cohort G: dose same as Cohort F, N=60

Dose frequency: single dose

Route of administration: intradermal injection using a West Intradermal Adaptor fitted to a fixed needle 1-mL insulin syringe

### Sample Size:

Approximately 138 patients

### Statistical Methods:

(1) Grading and duration of cytokine release syndrome and self-reported symptoms after administration of 30 μg, 45 μg, 60 μg, or 150 μg of gluten peptide composition are greater than placebo and suitable for a companion diagnostic in celiac disease patients on GFD.

(2) Gluten peptide-stimulated whole blood cytokine release is higher 6 days after administering the gluten peptide composition.

Data from each of the Cohorts A-D will be compared with Cohort E (placebo-treated) in the planned interim analysis.

In the final analysis of Cohorts F and G, placebo-treated subjects will be compared with composition-treated subjects.

Adverse events will be summarized for each dosing cohort, presenting the numbers and percent of patients having any adverse event (AE) and having AEs in each system organ class (SOC) and preferred term.

### Rationale:

This study is designed to optimize biomarker selection and establish a minimal dose of the gluten peptide composition that provokes a readily detectable increase in the biomarker. Dose ranging in Cohorts A-D will provide a single dose level to be studied in larger numbers of subjects in Cohort F and G. The current study will also provide greater understanding of factors that may influence or correlate with the magnitude of cytokine elevations and change in gene expression profiles in blood as well as clinical responses following a single dose of the gluten peptide composition: prior gluten exposure, compliance with GFD, intestinal tissue injury assessed by quantitative histology and gene expression profile, and pharmacogenetics. The highest dose level to be assessed in the current study is 150 μg and the lowest dose level to be assessed is 30 μg. Subjects in the current study will be monitored closely during the first six hours after dosing as this has been found to be when cytokine elevations and symptoms are most pronounced. Clinical review and further blood tests the following day and six days later will establish when normalization of cytokines occurs.

### Study Design (Further Details):

The first phase of the study (Cohorts A-E) is a double-blind dose ranging study to evaluate the immunological and clinical response to a single intra-dermal dose of the gluten peptide composition in HLA-DQ2.5+ patients with celiac disease on a GFD who are sero-negative for tTG IgA and not IgA deficient. The second phase of the study (Cohorts F and G) is a double-blind study evaluating the immunological and clinical response to a single intra-dermal fixed dose of the gluten peptide composition in patients with celiac disease on a GFD to correlate response with reported compliance with GFD, serum levels of tTG IgA, DGP IgG, and DGP IgA, intestinal injury measured by quantitative histology and by duodenal gene expression.

### Cohorts A-E

The study will consist of a Screening Period lasting up to six weeks followed by a one-day Treatment Period, and a 6-day Follow-up Period. The study will include four visits (one at Screening, one on the treatment day, and two during follow-up). Prior to screening, copies of medical reports shall be collected confirming intestinal villous atrophy and other laboratory and clinical abnormalities present when celiac disease was first diagnosed. At or within one week prior to the Screening visit, a self-administered dietary survey (Celiac Dietary Adherence Test, CDAT) will be completed to assess compliance with GFD (Leffler, Dennis et al. 2009). At the Screening visit, a medical history will be taken and a physical examination performed. Vital signs including HR, SBP, DBP, RR, SaO₂, and aural temperature will be recorded. An ECG will also be performed. At Screening, blood will be collected for the following: HLA-DQ genotyping to test for a panel of HLA-DQA and HLA-DQB alleles, recombinant human tTG IgA, and total IgA, full blood count with differential and platelet count, clotting, liver and renal function. Urinalysis will also be performed.

Patients meeting the entry criteria, who are also HLA-DQ2.5+ and sero-negative for tTG IgA and not IgA deficient will be randomized to receive a single intra-dermal dose of the gluten peptide composition: 30 μg (Cohort A), 45 μg (Cohort B), 60 μg (Cohort C), or 150 μg (Cohort D) or placebo (Cohort E). There will be eight subjects enrolled in each of Cohorts A-D, and sixteen in Cohort E. Randomization of subjects in Cohorts A-E will account for the requirement that two subjects in each of Cohort A-D and four subjects in Cohort E will have demonstrated no other HLA-DQA allele apart from HLA-DQA1*05 and no other HLA-DQB allele apart from HLA-DQB1*02 (“homozygous for HLA-DQ2.5”).

The day before treatment day, subjects will complete the GSRS according to the symptoms experienced during the prior one week. On the treatment day, subjects will arrive before 9 am after an overnight fast (no breakfast). The symptom diary applying to the previous 2 h will be completed prior to vital signs including HR, SBP, DBP, RR, SaO₂, and aural temperature being recorded. An ECG will also be performed. An 18-gauge intra-venous cannula will be inserted, preferably into a large antecubital vein. A 3-way tap will be attached and flushed with 2.5 mL of sterile normal saline containing heparin 10 U/mL. A loose tourniquet may be applied if necessary to facilitate blood collection, which will be preceded by withdrawing and discarding a volume of 2.5 mL. Blood is then collected via an adaptor directly into appropriate tubes for full blood count with differential and platelet count, clotting, liver and renal function, serum tTG-IgA, DGP-IgA, DGP-IgG, serum cytokines, CRP, whole blood RNA expression, and gluten peptide-stimulated whole blood cytokine release. After blood collection, the cannula will be flushed with 2.5 mL of sterile normal saline containing heparin 10 U/mL.

Gluten peptide composition or placebo will be administered in 0.1 mL using a syringe fitted with a West Intradermal Adaptor.

During the six hours after dosing vital signs (HR, SBP, DBP, RR, SaO₂, aural temperature) will be recorded, and blood will be collected hourly as described in the SOA (FIGS. 25A and 25B). Adverse events will be recorded after dosing. At 2 h, 4 h, and 6 h after dosing the symptom diary will be completed and apply to symptoms experienced in the previous two-hours, and a grading (0-5) for the severity of “Cytokine release syndrome” (MedDRA v.15 Code 10052015) since dosing will be recorded (see Appendix: Cytokine Release Syndrome Defined by Common Terminology Criteria for Adverse Events v4.03). After the assessments at 6 hours post-dose, the intra-venous cannula will be removed and the subject discharged from the study site.

The following day, subjects will return to the study site for assessments 23-25 h after dosing as described in the SOA.

The End-of-Study visit will be six days after dosing. Subjects will return to the study site for assessments as described in the SOA. A Gastrointestinal Symptom Rating Scale (GSRS) will be completed according the symptoms experienced during the prior one week since dosing.

### Cohorts F and G

After analysis of clinical and laboratory data derived from completed Cohorts A-E, a single dose level of the gluten peptide composition will be chosen and compared with placebo in Cohorts F and G. Subjects will be given the option to select whether they wish to enroll in Cohort E or Cohort F. The ratio of subjects randomized to receive the gluten peptide composition or placebo will be 2:1.

Subjects enrolled in Cohorts F and G will meet the same entry criteria as Cohorts A-E except that they will not be required to be sero-negative for tTG IgA, and will possess any of the HLA-DQA and HLA-DQB alleles associated with celiac disease (HLA-DQA1*05, HLA-DQB1*02, and HLA-DQB1*0302) not just HLA-DQ2.5.

Subjects may be enrolled in Cohorts F and G after being re-screened if they were excluded from enrolling in Cohorts A-E because tTG IgA serology was elevated, or because of IgA deficiency, or because they were not HLA-DQ2.5+, or because there was no further requirement in these cohorts for patients homozygous for HLA-DQ2.5 or for patients who were not homozygous for HLA-DQ2.5.

The Screening Visit for subjects in Cohorts F and G will be the same as for Cohorts A-E. Subjects who were previously screened for Cohorts A-E do not require repeat HLA-DQ genotyping, but all other screening tests and procedures will be repeated.

During the month prior to the treatment day, subjects in Cohort F will undergo an EGD for collection of six biopsies from the 2nd and 3rd parts of the duodenum. Quantitative histology will be performed on formalin-fixed biopsies to assess VH:CrD ratio and TEL density, and two biopsies will be collected into appropriate media for subsequent RNA expression profiling.

Procedures and assessments for Cohorts F and G will be otherwise the same as Cohorts A-E, except that vital signs, symptom diary, grading symptoms according to MedDRA v.15 Code 10052015 “cytokine release syndrome” and the FDA Guidance for Industry, “Toxicity Grading Scale for Healthy Adult and Adolescent Volunteers Enrolled in Preventive Vaccine Clinical Trials”, and blood collection will be limited to 4 h, 5 h, and 6 h after dosing.

### Clinical Laboratory Assessments:

Clinical laboratory tests (hematology and biochemistry) will be taken at several time points during the trial per the following schedule:
Screening
Baseline (Treatment Day, pre-dose)
During the 6 hours after dosing
Day 2
Day 6

Urinalysis will be performed via dipstick and a microscopic exam performed only if needed, depending on the result of the dipstick. Urine samples for urinalysis will be obtained at:
Screening
Baseline (Treatment Day, pre-dose)
Day 2
Day 6 (EOS)

Screening samples will be obtained under fasting conditions (no food or drink, except water, for at least 8 hours before sample collection). The laboratory tests to be performed are presented in the table below.

Clinical Laboratory Tests

| Category | Parameters |
|---|---|
| Hematology | Red blood cell (RBC) count, hemoglobin (Hb), |
| | hematocrit (Hct), platelets, and white blood cell (WBC) |
| | count with differential (bands, neutrophils, lymphocytes, |
| | monocytes, eosinophils, basophils) and platelet count |
| Coagulation | Prothrombin time (PT), and partial throboplastin time |
| | (PTT) |
| Chemistry | |
| Electrolytes | Sodium, potassium, chloride, bicarbonate (CO2) |
| Liver function tests | alkaline phosphatase, aspartate aminotransferase (AST), |
| | alanine aminotransferase (ALT), total bilirubin, direct |
| | bilirubin |
| Renal function | Blood urea/blood urea nitrogen (BUN), creatinine |
| parameters | |
| Other | Albumin, total protein, globulin, C-Reactive protein |
| | (CRP), and total serum IgA |
| Urinalysis | Urinalysis will be performed via dipstick and a |
| | microscopic exam performed only if needed, depending |
| | on the result of the dipstick. |
| Pregnancy Tests | Serum and urine beta human chorionic gonadotropin (β- |
| | hCG) for all female subjects |
| Viral Tests | Serum Hepatitis B (HBsAg), hepatitis C (HCV Ab), and |
| | human immunodeficiency virus (HIV) |
| Celiac Disease Serology | Serum recombinant human transglutaminase IgA, |
| | deamidated gliadin peptide IgG and deamidated gliadin |
| | peptide IgA |
| Cytokine Tests | Serum IL-2, IL-6, IL-8, IL-10, TNF-α, IP-10, MCP-1, |
| | eotaxin and additional cytokines and chemokines |
| | according to multiplex panels available |
| Genetic Tests | HLA-DQA and HLA-DQB allele analysis by |
| | polymerase chain reaction |
| Gene Expression Tests | Gene expression profiles will be determined by reverse |
| | transcription and amplification of RNA in whole blood |
| | or intestinal tissue collected into dedicated tubes |
| | containing an RNA-inhibitor. |

### Further Study Drug Information:

The gluten peptide compositions is provided as a 1.5 mg/mL stock and is supplied in sterile 2 ml vials for single-use with a concentration of each of the individual peptides at 0.5 mg/ml dissolved in sterile 0.9% sodium chloride. Vials are provided frozen and will be allowed to thaw at room temperature immediately prior to being prepared for administration to study subjects. The final injection volume of each syringe is 0.1 mL (100 μL). Sterile 0.9% sodium chloride for injection will be used to dilute the stock 1.5 mg/mL to the appropriate concentration for administration, and will also be used to prepare the placebo (0.9% sodium chloride).

The doses used are: 30 μg, 45 μg, 60 μg or 150 μg in syringes. Each patient will receive one intradermal dose containing the same dose volume (100 μL). To make each of the compositions, the following will be performed: 20 μL from the stock vial diluted with 80 μL 0.9% saline for the 30 μg dose; 30 μL from the stock vial diluted with 70 μL 0.9% saline for the 45 μg dose; 40 μL of from the stock vial diluted with 60 μL 0.9% saline for the 60 μg dose. For the 150 μg dose, 100 μL will be drawn from the stock 1.5 mg/mL vial. The placebo will be 100 μL 0.9% saline. The West Intradermal Adaptor will be fitted to the syringe immediately before the injection is administered to the patient.

### Primary Endpoints

At Interim Analysis after Completion of Cohorts A-E:

Grading and frequency of adverse events (AE's) after dosing with 30 μg (Cohort A), 45 μg (Cohort B), 60 μg (Cohort C) of the gluten peptide composition compared with 150 μg (Cohort D) of the gluten peptide composition and with placebo (Cohort E).

At Final Analysis after Completion of Cohorts F and G:

Grading and frequency of AE's in subjects administered a specified dose of the peptide composition compared to subjects dosed with placebo in Cohorts G and F.

Proportion of HLA-DQ2.5+ subjects with whole blood IP-10 release at EOS (Day 6) elevated above levels on Day 1 in subjects administered a specified dose of the peptide composition compared to those dosed with placebo in Cohorts G and F.

Proportion of HLA-DQ2.5+ subjects who are in remission as measured by tTG IgA serology, histology (VH:CrD), or closely adhere to GFD (CDAT score) with whole blood IP-10 release at EOS (Day 6) elevated above levels on Day 1 in subjects administered a specified dose of the gluten peptide composition compared to those who are not in remission, and subjects dosed with placebo in Cohorts G and F.

### REFERENCE LIST

(1990). “Revised criteria for diagnosis of coeliac disease. Report of Working Group of European Society of Paediatric Gastroenterology and Nutrition.” Arch Dis Child 65(8): 909-911.
(2007). Guidance for Industry. Toxicity Grading Scale for Healthy Adult and Adolescent Volunteers Enrolled in Preventive Vaccine Clinical Trials. U.S. D. o. H. a. H. Services. Rockville Md. U.S.A.
(2010). Common Terminology Criteria for Adverse Events (CTCAE) Version 4.03. U.S. D. O. H. A. H. SERVICES.
Anderson, R. P. (2005). “Coeliac disease.” Aust Fam Physician 34(4): 239-242.
Anderson, R. P., P. Degano, A. J. Godkin, D. P. Jewell and A. V. Hill (2000). “In vivo antigen challenge in celiac disease identifies a single transglutaminase-modified peptide as the dominant A-gliadin T-cell epitope.” Nat Med 6(3): 337-342.
Anderson, R. P., M. J. Henry, R. Taylor, E. L. Duncan, P. Danoy, M. J. Costa, K.
Addison, J. A. Tye-Din, M. A. Kotowicz, R. E. Knight, W. Pollock, G. C. Nicholson, B. H. Toh, M. A. Brown and J. A. Pasco (2013). “A novel serogenetic approach determines the community prevalence of celiac disease and informs improved diagnostic pathways.” BMC Med 11(1): 188.
Bardella, M. T., P. Velio, B. M. Cesana, L. Prampolini, G. Casella, C. Di Bella, A. Lanzini, M. Gambarotti, G. Bassotti and V. Villanacci (2007). “Coeliac disease: a histological follow-up study.” Histopathology 50(4): 465-471.
Bodd, M., C. Y. Kim, K. E. Lundin and L. M. Sollid (2012). “T-cell response to gluten in patients with HLA-DQ2.2 reveals requirement of peptide-MHC stability in celiac disease.” Gastroenterology 142(3): 552-561.
Brottveit, M., M. Raki, E. Bergseng, L. E. Fallang, B. Simonsen, A. Lovik, S. Larsen, E. M. Loberg, F. L. Jahnsen, L. M. Sollid and K. E. Lundin (2011). “Assessing possible celiac disease by an HLA-DQ2-gliadin Tetramer Test.” Am J Gastroenterol 106(7): 1318-1324.
Catassi, C., R. P. Anderson, I. D. Hill, S. Koletzko, E. Lionetti, N. Mouane, M. Schumann and S. K. Yachha (2012). “World perspective on celiac disease.” J Pediatr Gastroenterol Nutr 55(5): 494-499.
Chen, Z., N. Dudek, O. Wijburg, R. Strugnell, L. Brown, G. Deliyannis, D. Jackson, F. Koentgen, T. Gordon and J. McCluskey (2002). “A 320-kilobase artificial chromosome encoding the human HLA DR3-DQ2 MHC haplotype confers HLA restriction in transgenic mice.” J Immunol 168(6): 3050-3056.
de Kauwe, A. L., Z. Chen, R. P. Anderson, C. L. Keech, J. D. Price, O. Wijburg, D. C. Jackson, J. Ladhams, J. Allison and J. McCluskey (2009). “Resistance to celiac disease in humanized HLA-DR3-DQ2-transgenic mice expressing specific anti-gliadin CD4+ T cells.” J Immunol 182(12): 7440-7450.
Gianfrani, C., R. Troncone, P. Mugione, E. Cosentini, M. De Pascale, C. Faruolo, S. Senger, G. Terrazzano, S. Southwood, S. Auricchio and A. Sette (2003). “Celiac disease association with CD8+ T cell responses: identification of a novel gliadin-derived HLA-A2-restricted epitope.” J Immunol 170(5): 2719-2726.
Henderson, K. N., J. A. Tye-Din, H. H. Reid, Z. Chen, N. A. Borg, T. Beissbarth, A. Tatham, S. I. Mannering, A. W. Purcell, N. L. Dudek, D. A. van Heel, J. McCluskey, J. Rossjohn and R. P. Anderson (2007). “A structural and immunological basis for the role of human leukocyte antigen DQ8 in celiac disease.” Immunity 27(1): 23-34.
Jabri, B. and L. M. Sollid (2009). “Tissue-mediated control of immunopathology in coeliac disease.” Nat Rev Immunol 9(12): 858-870.
Karell, K., A. S. Louka, S. J. Moodie, H. Ascher, F. Clot, L. Greco, P. J. Ciclitira, L. M. Sollid, J. Partanen and D. European Genetics Cluster on Celiac (2003). “HLA types in celiac disease patients not carrying the DQA1*05-DQB1*02 (DQ2) heterodimer: results from the European Genetics Cluster on Celiac Disease.” Hum Immunol 64(4): 469-477.
Kaukinen, K., M. Peraaho, K. Lindfors, J. Partanen, N. Woolley, P. Pikkarainen, A. L. Karvonen, T. Laasanen, H. Sievanen, M. Maki and P. Collin (2007). “Persistent small bowel mucosal villous atrophy without symptoms in coeliac disease.” Aliment Pharmacol Ther 25(10): 1237-1245.
Keech, C. L., Mcluskey, J. (2008). Bioactivity assessment of Nexvax2 in a relevant in vivo mouse model: dose response, University of Melbourne.
Keech, C. L., Mcluskey, J. (2008). Bioactivity assessment of Nexvax2 in a relevant in vivo mouse model: immune modulation following repeat administration of Nexvax2, University of Melbourne.
Keech, C. L., Mcluskey, J. (2008). Bioactivity assessment of Nexvax2 in a relevant in vivo mouse model: response of antigen primed gliadin specific T cells., University of Melbourne.
Keech, C. L., Mcluskey, J. (2008). Bioactivity assessment of Nexvax2 in a relevant in vivo mouse model: response of nave gliadin specific T cells., University of Melbourne.
Keech, C. L., Mcluskey, J. (2010). Bioactivity assessment of Nexvax2 in a relevant in vivo mouse model, University of Melbourne.
Keech, C. L., Mcluskey, J. (2011). Bioactivity assessment of Nexvax2 in a relevant in vivo mouse model., University of Melbourne.
Koskinen, L., J. Romanos, K. Kaukinen, K. Mustalahti, I. Korponay-Szabo, D. Barisani, M. T. Bardella, F. Ziberna, S. Vatta, G. Szeles, Z. Pocsai, K. Karell, K. Haimila, R. Adany, T. Not, A. Ventura, M. Maki, J. Partanen, C. Wijmenga and P. Saavalainen (2009). “Cost-effective HLA typing with tagging SNPs predicts celiac disease risk haplotypes in the Finnish, Hungarian, and Italian populations.” Immunogenetics 61(4): 247-256.
Leffler, D., D. Schuppan, K. Pallav, R. Najarian, J. D. Goldsmith, J. Hansen, T. Kabbani, M. Dennis and C. P. Kelly (2013). “Kinetics of the histological, serological and symptomatic responses to gluten challenge in adults with coeliac disease.” Gut 62(7): 996-1004.
Leffler, D. A., M. Dennis, J. B. Edwards George, S. Jamma, S. Magge, E. F. Cook, D. Schuppan and C. P. Kelly (2009). “A simple validated gluten-free diet adherence survey for adults with celiac disease.” Clin Gastroenterol Hepatol 7(5): 530-536, 536 e531-532.
Liu, E., H. S. Lee, C. A. Aronsson, W. A. Hagopian, S. Koletzko, M. J. Rewers, G. S. Eisenbarth, P. J. Bingley, E. Bonifacio, V. Simell, D. Agardh and T. S. Group (2014). “Risk of pediatric celiac disease according to HLA haplotype and country.” N Engl J Med 371(1): 42-49.
Murray, J. A., T. Watson, B. Clearman and F. Mitros (2004). “Effect of a gluten-free diet on gastrointestinal symptoms in celiac disease.” Am J Clin Nutr 79(4): 669-673.
Ontiveros, N., J. A. Tye-Din, M. Y. Hardy and R. P. Anderson (2014). “Ex-vivo whole blood secretion of interferon (IFN)-gamma and IFN-gamma-inducible protein-10 measured by enzyme-linked immunosorbent assay are as sensitive as IFN-gamma enzyme-linked immunospot for the detection of gluten-reactive T cells in human leucocyte antigen (HLA)-DQ2.5(+)-associated coeliac disease.” Clin Exp Immunol 175(2): 305-315.
Raki, M., L. E. Fallang, M. Brottveit, E. Bergseng, H. Quarsten, K. E. Lundin and L. M. Sollid (2007). “Tetramer visualization of gut-homing gluten-specific T cells in the peripheral blood of celiac disease patients.” Proc Natl Acad Sci USA. 104(8):2831-6.
Simell, S., S. Hoppu, A. Hekkala, T. Simell, M. R. Stahlberg, M. Viander, H. Yrjanainen, J. Gronlund, P. Markula, V. Simell, M. Knip, J. Ilonen, H. Hyoty and O. Simell (2007). “Fate of five celiac disease-associated antibodies during normal diet in genetically at-risk children observed from birth in a natural history study.” Am J Gastroenterol 102(9): 2026-2035.
Simell, S., A. Kupila, S. Hoppu, A. Hekkala, T. Simell, M. R. Stahlberg, M. Viander,
T. Hurme, M. Knip, J. Ilonen, H. Hyoty and O. Simell (2005). “Natural history of transglutaminase autoantibodies and mucosal changes in children carrying HLA-conferred celiac disease susceptibility.” Scand J Gastroenterol 40(10): 1182-1191.
Sollid, L. M., S. W. Qiao, R. P. Anderson, C. Gianfrani and F. Koning (2012). “Nomenclature and listing of celiac disease relevant gluten T-cell epitopes restricted by HLA-DQ molecules.” Immunogenetics 64(6): 455-460.
Tye-Din, J. A., R. P. Anderson, R. A. Ffrench, G. J. Brown, P. Hodsman, M. Siegel, W. Botwick and R. Shreeniwas (2010). “The effects of ALV003 pre-digestion of gluten on immune response and symptoms in celiac disease in vivo.” Clin Immunol 134(3): 289-295.
Vader, W., D. Stepniak, Y. Kooy, L. Mearin, A. Thompson, J. J. van Rood, L. Spaenij and F. Koning (2003). “The HLA-DQ2 gene dose effect in celiac disease is directly related to the magnitude and breadth of gluten-specific T cell responses.” Proc Natl Acad Sci USA 100(21): 12390-12395.

### Example 5

### Whole Blood Cytokine Release Stimulated by Gluten Peptides in Seronegative CD Patients Compared to Seronegative Patients with Non-Celiac Gluten Sensitivity with Reduced Intake of Dietary Gluten

Aim: To assess gluten-peptide pool stimulated whole blood cytokine release assays for celiac disease (CD) patients negative for CD-specific serology (tTG-IgA and DGP-IgG).

### Endpoints:

### Primary

Sensitivity and specificity of whole blood cytokine release detected by IP-10 ELISA for tTG-IgA/DGP-IgG seronegative CD vs non-celiac gluten-sensitive (NCGS) patients

### Secondary

(1) Sensitivity and specificity of whole blood cytokine release detected by IP-10 ELISA for CD patients vs NCGS patients who carry HLA-DQ genotypes associated with celiac disease (HLA-DQ2.5+ or DQ2.2+ or DQ8+).

### Patients:

### Inclusion:

(1) Celiac disease on gluten-free diet diagnosis of CD established and documented according to Expert Clinical Guidelines (e.g. World Gastroenterology Organisation Global Guidelines on Celiac Disease. 2013) who self report being generally compliant with gluten-free diet

### OR

Non-celiac gluten-sensitive established by normal tTG-IgA serology and/or small bowel histology while regularly consuming gluten who self report being generally compliant with gluten-free diet
(2) No medical contradiction to blood collection by standard venepuncture with a 21G butterfly needle
(3) tTG-IgA (INOVA rhtTG-IgA) and DGP-IgG (INOVA Gliaden II IgG) within the laboratory normal range
(4) Aged 18 or older

### Screening Tests and Information:

EDTA-anticoagulated blood for comprehensive HLA-DQA and HLA-DQB allele determination
Serum tTG-IgA (INOVA rhtTG-IgA) and DGP-IgG (INOVA Gliaden II IgG) assessment
Documentation of medical tests establishing or excluding a diagnosis of celiac disease
Symptoms at diagnosis and current GI symptoms
Duration of gluten-free diet
Co-morbidities (if any)
Medications (if any)

### Age and sex

### Procedure:

Subjects attend a single visit to the trial site for collection of blood to perform:
1. HLA-DQ gene test (Lavender-top EDTA 5 mL, Melbourne Pathology, SONIC)
2. CD serology (Brown-top serum tube 5 mL, Dorevitch Pathology),
3. Whole blood release—subjects will have ONE tube (1 mL blood/tube) for each whole blood incubation condition (9 Quantiferon-GoldTB NIL and 1 MITO tube). In addition, 10 of 30 CD subjects will have 27 additional Cellestis NIL tubes drawn to determine inter- and intra-assay variability (the first 10 CD subjects). After blood is drawn, 0.1 mL volumes of aliquots (listed below) are added by 0.5 mL insulin syringe to NIL tubes containing 1 mL blood, and PBS is added to MITOGEN tube containing 1 mL blood. All Quantiferon tubes are placed in 37° C. incubator. After 24 h incubation, plasma is separated from blood in the Quantiferon tubes and placed in appropriately labeled cryovials then frozen −80° C. Frozen plasma samples then used for ELISA determination of IP-10.
   Tubes and aliquots are prepared containing one of the following:

### PBS

### PBS+0.5% DMSO,

CEFT 11 ug/mL,
Pool 1—P3 pool 550 μg/mL in PBS (see Example 3 for P3 pool peptides)
Pool 2—P14 pool 2751.1M in PBS (see Example 3 for P14 pool peptides)
Pool 3—Total Gluten 110 pg/mL in PBS 0.5% DMSO
Pool 4a—P16 pool 1101.1M in PBS
Pool 4b—P16 pool 2751.1M in PBS
Pool 4c—P16 pool 5501.1M in PBS

P16 pool

| Peptide | Epitope(s) |
|---|---|
| (pE)PFPQPELPYPQP-amide (SEQ ID NO: | PFPQPELPY (SEQ ID NO: 1), |
| 286) | PQPELPYPQ (SEQ ID NO: 2) |
| (pE)PFPQPEQPFPWQ-amide (SEQ ID NO: | PFPQPEQPF (SEQ ID NO: 3), |
| 287) | PQPEQPFPW (SEQ ID NO: 4) |
| (pE)EQPIPEQPQPYP-amide (SEQ ID NO: | EQPIPEQPQ (SEQ ID NO: 5), |
| 288) | PIPEQPQPY (SEQ ID NO: 6) |
| (pE)PFPQPEQPIPVQ-amide (SEQ | PFPQPEQPI (SEQ ID NO: 7), |
| ID NO: 289) | PQPEQPIPV (SEQ ID NO: 8) |
| (pE)PEQPIPVQPEQS-amide (SEQ | EQPIPVQPE (SEQ ID NO: 9) |
| ID NO: 290) | |
| (pE)PFPQPEQPTPIQ-amide (SEQ | PFPQPEQPT (SEQ ID NO: 10), |
| ID NO: 291) | PQPEQPTPI (SEQ ID NO: 11) |
| (pE)PEQPTPIQPEQP-amide (SEQ | EQPTPIQPE (SEQ ID NO: 12) |
| ID NO: 292) | |
| (pE)PFPQPEQPFPLQ-amide (SEQ | PQPEQPFPL (SEQ ID NO: 13) |
| ID NO: 293) | |
| (pE)PEQPFPLQPEQP-amide (SEQ | EQPFPLQPE (SEQ ID NO: 14) |
| ID NO: 294) | |
| (pE)GEGSFQPSQENP-amide | EGSFQPSQE (SEQ ID NO: 15) |
| (SEQ ID NO: 295) | |
| (pE)QQGYYPTSPQQS-amide | QGYYPTSPQ (SEQ ID NO: 16) |
| (SEQ ID NO: 296) | |
| (pE)PEQPEQPFPEQP-amide (SEQ | EQPEQPFPE (SEQ ID NO: 17) |
| ID NO: 297) | |
| (pE)PPFSEQEQPVLP-amide (SEQ | PFSEQEQPV (SEQ ID NO: 18) |
| ID NO: 298) | |
| (pE)PYPQPELPYPQP-amide (SEQ | PYPQPELPY (SEQ ID NO: 19), |
| ID NO: 299) | (PQPELPYPQ, SEQ ID NO: 2) |
| (pE)EQPFPEQPIPEQ-amide (SEQ | EQPFPEQPI (SEQ ID NO: 20), |
| ID NO: 300) | PFPEQPIPE (SEQ ID NO: 21) |
| (pE)PQPYPEQPQPFP-amide (SEQ | PYPEQPQPF (SEQ ID NO: 22), |
| ID NO: 301) | PQPYPEQPQ (SEQ ID NO: 23) |

(pE) = pyroglutamate

Validated ELISAs and/or bead-based multiplex assays will be used for determination of IP-10 and IFN-γ and will be used to establish an upper limit for stimulation index and concentration of each analyte using plasma collected from NCGS who are not genetically susceptible to celiac disease. In the initial analysis, data points will be determined to be elevated or not according to this threshold (e.g. Stimulated blood minus NIL with PBS only >7.2 pg/mL and Stimulated blood/NIL with PBS only >1.25 for IFNγ). Threshold values to optimize sensitivity and specificity differentiating CD vs NCGS will be further refined according receiver operating characteristic (ROC) curve analysis and area under the curve (AUC) analysis. Data from subjects with CD who are excluded because of being seropositive for tTG-IgA or DGP-IgG will be reported and analyzed separately according to the same cutoffs as applied to other subjects.

### Sample Size Estimation

Celiac disease—approximately ⅓ of treated CD subjects show elevated CD-serology and >99% are HLA-DQ2.5+ or DQ8+ or DQ2.2+
NCGS—all have normal CD serology and 60% are HLA-DQ2.5+ or DQ8+ or DQ2.2+
To enroll ˜20 seronegative CD subjects, 30 total should be enrolled
To enroll ˜20 HLA-DQ2.5+ or DQ8+ or DQ2.2+ NCGS subjects, 30 total should enrolled

### Example 6

### Whole Blood Cytokine Release Stimulated by Gluten Peptides in Seronegative CD Patients

### Aim:

To determine 24 h IP-10 release stimulated by gluten-peptide pools in whole blood collected from subjects with celiac disease (CD) on strict gluten-free diet who were negative for celiac disease-specific serology (tTG-IgA and DGP-IgG).

Subjects were included if they met the following criteria:

(1) Celiac disease on gluten-free diet diagnosis of CD established and documented according to Expert Clinical Guidelines (e.g. World Gastroenterology Organisation Global Guidelines on Celiac Disease. 2013) who self-report being generally compliant with gluten-free diet

(2) No medical contradiction to blood collection by standard venepuncture with a 21G butterfly needle

(3) tTG-IgA (INOVA rhtTG-IgA) and DGP-IgG (INOVA Gliaden II IgG) within the laboratory normal range

(4) Aged 18-70 years

(5) Provide written informed consent

### Procedure:

Blood (1 mL) was collected directly into heparinized tubes and peptide solutions in phosphate buffered saline (PBS) or PBS alone (0.1 mL) was injected into the blood collection tube. Blood was incubated for 24 h at 37° C. After incubation, plasma was separated and frozen until being thawed and the concentration of IP-10 determined by magnetic bead assay (Luminex, Millipore). Peptide solutions consisted of the following from Example 3: Gluten Pool 1 (2 16mers and 1 15mer peptides), Pool 2 (14 peptides 13-19mers) and Pool 4 (16 13mer peptides). Pool 1 included at least five HLA-DQ2.5-restricted epitopes, Pool 2 and Pool 4 were designed to include the same core 9mer sequences recognized by gluten-reactive CD4+ T cells from HLA-DQ2.5+, HLA-DQ8+, and/or HLA-DQ2.2+ donors with celiac disease.

### Results:

Demographics of subjects included in the study are shown in the below table. Seven donors aged between 35 and 56 yrs were studied, five were HLA-DQ2.5+, and the two other subjects were either HLA-DQ8+ or HLA-DQ2.2+.

Demographics of subjects

| | | | | | | CD | | | | |
|---|---|---|---|---|---|---|---|---|---|---|
| | HLA serotype | | | | | Serology | | Year of | | |
| | HLA- | HLA- | HLA- | HLA alleles | | tTg- | DGP- | CD | GFD | |
| Subject | DQ | DR | DQ | DQA1 | DQB1 | IgA | IgG | diagnosis | since | Age/yrs |
| A | 2.5, 2.2 | 3 7 | 2, 2 | 02 05 | 02 02 | <1 | 1 | 2002 | 2002 | 49 |
| B | 2.5 | 3 3 | 2, 2 | 05 05 | 02 02 | 1 | 4 | 2009 | 2009 | 56 |
| | homozygote | | | | | | | | | |
| C | 2.5 | 1 3 | 2, 5 | 01 05 | 02 05 | <1 | 5 | 1990 | 1990 | 51 |
| D | 2.5 | 3 15 | 2, 6 | 01 05 | 02 06 | <1 | 3 | 2010 | 2010 | 44 |
| E | 2.5/8 | 3 4 | 2, 8 | 03 05 | 02 0302 | 3 | 98 | 2003 | 2004 | 49 |
| F | 8 | 4 13 | 6, 8 | 01, 03 | 0302, 06 | 1 | 2 | 2010 | 2010 | 35 |
| G | 2.2 | 7 13 | 2, 6 | 01, 02 | 02, 06 | <1 | 2 | 2004 | 2004 | 50 |

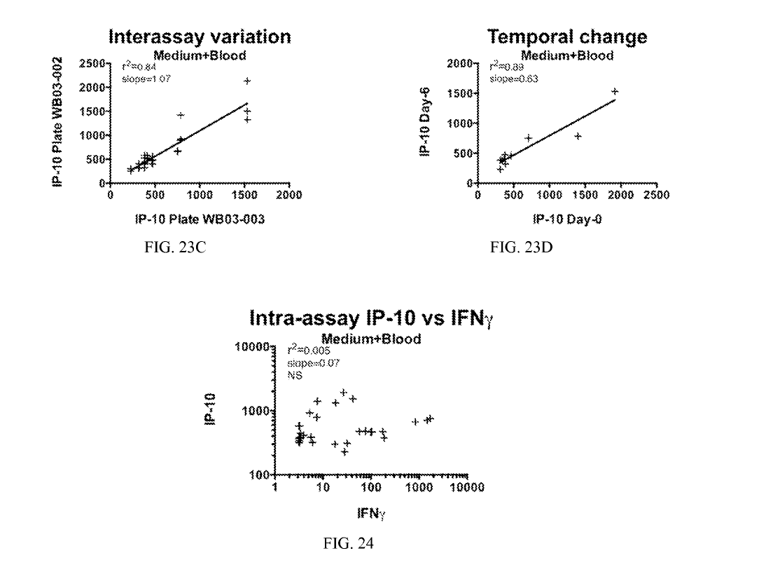

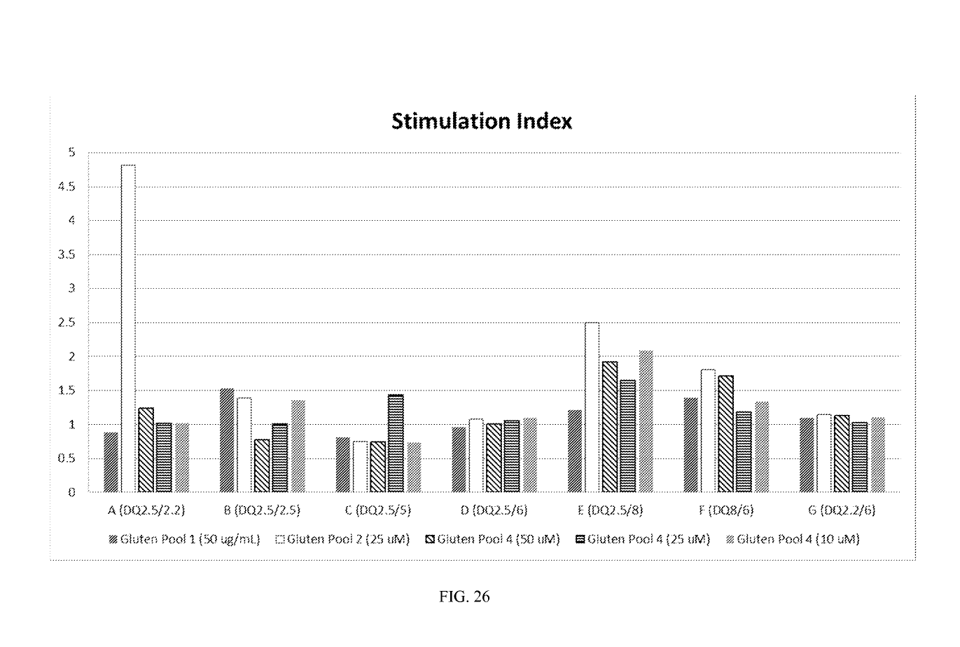

Peptide Pool 2 and Pool 4 showed stimulation indices higher than Pool 1, and above one, i.e. IP-10 plasma concentrations were greater than in blood incubated with PBS alone (FIG. 23). Notably, the subject with the genotype HLA-DQ8/6+ showed a stimulation index above one for Pool 2 and Pool 4, but not Pool 1 indicating the potential for the expanded peptide pools to activate T cells in donors who were not HLA-DQ2.5+(FIG. 26).

### Conclusion:

Expanded peptide pools that include gluten-derived epitopes additional to those represented in the 3-peptide composition (PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), and EQPIPEQPQ (SEQ ID NO: 5)) have, in some embodiments, the capacity to increase IP-10 release in blood from celiac disease donors who are HLA-DQ2.5+ and those who are negative for HLA-DQ2.5.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of identifying a subject having or at risk for having celiac disease, the method comprising:
(a) determining a level of IP-10 in a sample comprising a T cell from the subject, which sample has been contacted with a composition comprising at least one gluten peptide; and
(b) assessing whether or not the subject has or is at risk of having Celiac disease.

2. The method of claim 1, wherein the determining step comprises:
(i) contacting the sample comprising the T cell with the composition comprising at least one gluten peptide; and
(ii) measuring the level of IP-10 in the sample comprising the T cell after the contacting.

3. The method of claim 2, wherein measuring the level of IP-10 comprises an enzyme-linked immunosorbent assay (ELISA) or a multiplex bead-based immunoassay.

4. The method of any one of claims 1 to 3, wherein the method further comprises:
(c) comparing the level of IP-10 in the sample with a control level of IP-10.

5. The method of any one of claims 1 to 4, wherein the assessing comprises:
(i) identifying the subject as having or at risk of having Celiac disease if the IP-10 level is elevated compared to a control level of IP-10; or
(ii) not having or not at risk of having Celiac disease if the IP-10 level is reduced compared to the control level of IP-10 or the same as the control level of IP-10.

6. The method of claim 5, wherein the control level of IP-10 is a pre-determined threshold.

7. The method of claim 5, wherein the control level of IP-10 is the level of IP-10 in another sample comprising a T cell obtained from the subject that is not contacted with the composition comprising at least one gluten peptide.

8. The method of any one claims 1 to 7, wherein the sample comprising the T cell is a sample that comprises whole blood or peripheral blood mononuclear cells.

9. The method of any one of claims 1 to 8, wherein the composition comprises at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 18), EQPFPEQPI (SEQ ID NO: 20), PFPEQPIPE (SEQ ID NO: 21), PYPQPELPY (SEQ ID NO: 19), PQPELPYPY (SEQ ID NO: 43), and PQPYPEQPQ (SEQ ID NO: 23).

10. The method of any one of claims 1 to 8, wherein the composition comprises at least one of
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);
(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);
(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 40);
(h) an eighth peptide comprising the amino acid sequence PYPEQPQPF (SEQ ID NO: 22);
(i) a ninth peptide comprising the amino acid sequence PFPEQPEQI (SEQ ID NO: 42);
(j) a tenth peptide comprising the amino acid sequence EGSFQPSQE (SEQ ID NO: 15);
(k) an eleventh peptide comprising the amino acid sequence QGYYPTSPQ (SEQ ID NO: 16);
(l) a twelfth peptide comprising the amino acid sequence EQPEQPFPE (SEQ ID NO: 17) and the amino acid sequence EQPFPEQPQ (SEQ ID NO: 41);
(m) a thirteenth peptide comprising the amino acid sequence PFSEQEQPV (SEQ ID NO: 18);
(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 19);
(p) a sixteenth peptide comprising the amino acid sequence PFPQPELPY (SEQ ID NO: 1) and the amino acid sequence PQPELPYPY (SEQ ID NO: 43);
(q) a seventeenth peptide comprising the amino acid sequence EQPFPEQPI (SEQ ID NO: 20) and the amino acid sequence PFPEQPIPE (SEQ ID NO: 21); and
(r) an eighteenth peptide comprising the amino acid sequence PQPYPEQPQ (SEQ ID NO: 23) and the amino acid sequence PYPEQPQPF (SEQ ID NO: 22).

11. The method of claim 10, wherein:
(a) the first peptide comprises the amino acid sequence LQPFPQPELPYPQPQ (SEQ ID NO: 45);
(b) the second peptide comprises the amino acid sequence QPFPQPEQPFPWQP (SEQ ID NO: 46);
(c) the third peptide comprises the amino acid sequence PEQPIPEQPQPYPQQ (SEQ ID NO: 47);
(d) the fourth peptide comprises the amino acid sequence QPFPQPEQPIPVQPEQS (SEQ ID NO: 48);
(e) the fifth peptide comprises the amino acid sequence QPFPQPEQPTPIQPEQP (SEQ ID NO: 49);
(f) the sixth peptide comprises the amino acid sequence QPFPQPEQPFPLQPEQP (SEQ ID NO: 50);
(g) the seventh peptide comprises the amino acid sequence QPFPQPEQPFSQQ (SEQ ID NO: 51);
(h) the eighth peptide comprises the amino acid sequence PQPYPEQPQPFPQQ (SEQ ID NO: 52);
(i) the ninth peptide comprises the amino acid sequence QPFPEQPEQIIPQQP (SEQ ID NO: 53);
(j) the tenth peptide comprises the amino acid sequence SGEGSFQPSQENPQ (SEQ ID NO: 54);
(k) the eleventh peptide comprises the amino acid sequence GQQGYYPTSPQQSG (SEQ ID NO: 55);
(l) the twelfth peptide comprises the amino acid sequence PEQPEQPFPEQPQQ (SEQ ID NO: 56);
(m) the thirteenth peptide comprises the amino acid sequence QPPFSEQEQPVLPQ (SEQ ID NO: 57);
(n) the fourteenth peptide comprises the amino acid sequence PEQPFPEQPIPEQPQPYP (SEQ ID NO: 44);
(o) the fifteenth peptide comprises the amino acid sequence QPYPQPELPYPQPQ (SEQ ID NO: 58);
(p) the sixteenth peptide comprises the amino acid sequence QPFPQPELPYPYPQ (SEQ ID NO: 59);
(q) the seventeenth peptide comprises the amino acid sequence PQEQPFPEQPIPEQP (SEQ ID NO: 60); and
(r) the eighteenth peptide comprises the amino acid sequence QPQPYPEQPQPFPQQ (SEQ ID NO: 61).

12. The method of any one of claims 1 to 9, wherein the composition comprises at least one of:
(i) a first peptide comprising the amino acid sequence PFPQPELPY (SEQ ID NO: 1) and the amino acid sequence PQPELPYPQ (SEQ ID NO: 2);
(ii) a second peptide comprising the amino acid sequence PFPQPEQPF (SEQ ID NO: 3) and the amino acid sequence PQPEQPFPW (SEQ ID NO: 4);
(iii) a third peptide comprising the amino acid sequence PIPEQPQPY (SEQ ID NO: 6);
(iv) a fourth peptide comprising the amino acid sequence PFPQPEQPIP (SEQ ID NO: 62) and the amino acid sequence EQPIPVQPE (SEQ ID NO: 9);
(v) a fifth peptide comprising the amino acid sequence PFPQPEQPTPI (SEQ ID NO: 63) and the amino acid sequence EQPTPIQPE (SEQ ID NO: 12);
(vi) a sixth peptide comprising the amino acid sequence PQPEQPFPL (SEQ ID NO: 13) and the amino acid sequence EQPFPLQPE (SEQ ID NO: 14);
(vii) a seventh peptide comprising the amino acid sequence PFPQPEQPF (SEQ ID NO: 3) and the amino acid sequence PQPEQPFSQ (SEQ ID NO: 40);
(viii) an eighth peptide comprising the amino acid sequence PYPEQPQPF (SEQ ID NO: 22);
(ix) a ninth peptide comprising the amino acid sequence PFPEQPEQIIP (SEQ ID NO: 64);
(x) a tenth peptide comprising the amino acid sequence EGSFQPSQE (SEQ ID NO: 15);
(xi) an eleventh peptide comprising the amino acid sequence QGYYPTSPQ (SEQ ID NO: 16);
(xii) a twelfth peptide comprising the amino acid sequence EQPEQPFPEQPQ (SEQ ID NO: 65);
(xiii) a thirteenth peptide comprising the amino acid sequence PFSEQEQPV (SEQ ID NO: 18);
(xiv) a fourteenth peptide comprising the amino acid sequence EQPFPEQPI (SEQ ID NO: 20) and PIPEQPQPY (SEQ ID NO: 6);
(xv) a fifteenth peptide comprising the amino acid sequence PQPELPYPQ (SEQ ID NO: 2) and the amino acid sequence PYPQPELPY (SEQ ID NO: 19);
(xvi) a sixteenth peptide comprising the amino acid sequence PFPQPELPY (SEQ ID NO: 1) and the amino acid sequence PQPELPYPY (SEQ ID NO: 43); and
(xvii) a seventeenth peptide comprising the amino acid sequence EQPFPEQPI (SEQ ID NO: 20).

13. The method of claim 12, wherein
(i) the first peptide comprises the amino acid sequence
(SEQ ID NO: 45)
LQPFPQPELPYPQPQ;
(ii) the second peptide comprises the amino acid sequence
(SEQ ID NO: 46)
QPFPQPEQPFPWQP;
(iii) the third peptide comprises the amino acid sequence
(SEQ ID NO: 47)
PEQPIPEQPQPYPQQ;
(iv) the fourth peptide comprises the amino acid sequence
(SEQ ID NO: 48)
QPFPQPEQPIPVQPEQS;
(v) the fifth peptide comprises the amino acid sequence
(SEQ ID NO: 49)
QPFPQPEQPTPIQPEQP;
(vi) the sixth peptide comprises the amino acid sequence
(SEQ ID NO: 50)
QPFPQPEQPFPLQPEQP;
(vii) the seventh peptide comprises the amino acid sequence
(SEQ ID NO: 51)
QPFPQPEQPFSQQ;
(viii) the eighth peptide comprises the amino acid sequence
(SEQ ID NO: 52)
PQPYPEQPQPFPQQ;
(ix) the ninth peptide comprises the amino acid sequence
(SEQ ID NO: 53)
QPFPEQPEQIIPQQP;
(x) the tenth peptide comprises the amino acid sequence
(SEQ ID NO: 54)
SGEGSFQPSQENPQ;
(xi) the eleventh peptide comprises the amino acid sequence
(SEQ ID NO: 55)
GQQGYYPTSPQQSG;
(xii) the twelfth peptide comprises the amino acid sequence
(SEQ ID NO: 56)
PEQPEQPFPEQPQQ;
(xiii) the thirteenth peptide comprises the amino acid sequence
(SEQ ID NO: 57)
QPPFSEQEQPVLPQ;
(xiv) the fourteenth peptide comprises the amino acid sequence
(SEQ ID NO: 44)
PEQPFPEQPIPEQPQPYP;
(xv) the fifteenth peptide comprises the amino acid sequence
(SEQ ID NO: 58)
QPYPQPELPYPQPQ;
(xvi) the sixteenth peptide comprises the amino acid sequence
(SEQ ID NO: 59)
QPFPQPELPYPYPQ;
and
(xvii) a seventeenth peptide comprises the amino acid sequence EQPFPEQPI (SEQ ID NO: 20).

14. The method of any one of claims 10 to 13, wherein the composition comprises the first, second, and third peptide.

15. The method of any one of claims 10 to 13, wherein the composition comprises the first, second, third, fourth, fifth, sixth, seventh, eighth, ninth, tenth, eleventh, twelfth, and thirteenth peptides.

16. The method of any one of claims 10 to 13, wherein the composition comprises the second, fourth, fifth, sixth, seventh, eighth, ninth, tenth, eleventh, twelfth, thirteenth, fourteenth, fifteenth, and sixteenth peptides.

17. The method of claim 10 or 11, wherein the composition comprises the first, second, third, fourth, fifth, sixth, tenth, eleventh, twelfth, thirteenth, fifteenth, seventeenth, and eighteenth peptides.

18. The method of any one of claims 1 to 9, wherein the composition comprises at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7) and the amino acid sequence PQPEQPIPV (SEQ ID NO: 8);
(e) a fifth peptide comprising the amino acid sequence EQPIPVQPE (SEQ ID NO: 9);
(f) a sixth peptide comprising the amino acid sequence PFPQPEQPT (SEQ ID NO: 10) and the amino acid sequence PQPEQPTPI (SEQ ID NO: 11);
(g) a seventh peptide comprising the amino acid sequence EQPTPIQPE (SEQ ID NO: 12);
(h) an eighth peptide comprising the amino acid sequence PQPEQPFPL (SEQ ID NO: 13);
(i) a ninth peptide comprising the amino acid sequence EQPFPLQPE (SEQ ID NO: 14);
(j) a tenth peptide comprising the amino acid sequence EGSFQPSQE (SEQ ID NO: 15);
(k) a eleventh peptide comprising the amino acid sequence QGYYPTSPQ (SEQ ID NO: 16);
(l) a twelfth peptide comprising the amino acid sequence EQPEQPFPE (SEQ ID NO: 17);
(m) a thirteenth peptide comprising the amino acid sequence PFSEQEQPV (SEQ ID NO: 18);
(n) a fourteenth peptide comprising the amino acid sequence PYPQPELPY (SEQ ID NO: 19);
(o) a fifteenth peptide comprising the amino acid sequence EQPFPEQPI (SEQ ID NO: 20) and the amino acid sequence PFPEQPIPE (SEQ ID NO: 21); and
(p) a sixteenth peptide comprising the amino acid sequence PYPEQPQPF (SEQ ID NO: 22) and the amino acid sequence PQPYPEQPQ (SEQ ID NO: 23).

19. The method of claim 18, wherein
(a) the first peptide comprises the amino acid sequence PFPQPELPYPQP (SEQ ID NO: 24);
(b) the second peptide comprises the amino acid sequence PFPQPEQPFPWQ (SEQ ID NO: 25);
(c) the third peptide comprises the amino acid sequence EQPIPEQPQPYP (SEQ ID NO: 26);
(d) the fourth peptide comprises the amino acid sequence PFPQPEQPIPVQ (SEQ ID NO: 27);
(e) the fifth peptide comprises the amino acid sequence PEQPIPVQPEQS (SEQ ID NO: 28);
(f) the sixth peptide comprises the amino acid sequence PFPQPEQPTPIQ (SEQ ID NO: 29);
(g) the seventh peptide comprises the amino acid sequence PEQPTPIQPEQP (SEQ ID NO: 30);
(h) the eighth peptide comprises the amino acid sequence PFPQPEQPFPLQ (SEQ ID NO: 31);
(i) the ninth peptide comprises the amino acid sequence PEQPFPLQPEQP (SEQ ID NO: 32);
(j) the tenth peptide comprises the amino acid sequence GEGSFQPSQENP (SEQ ID NO: 33);
(k) the eleventh peptide comprises the amino acid sequence QQGYYPTSPQQS (SEQ ID NO: 34);
(l) the twelfth peptide comprises the amino acid sequence PEQPEQPFPEQP (SEQ ID NO: 35);
(m) the thirteenth peptide comprises the amino acid sequence PPFSEQEQPVLP (SEQ ID NO: 36);
(n) the fourteenth peptide comprises the amino acid sequence PYPQPELPYPQP (SEQ ID NO: 37);
(o) the fifteenth peptide comprises the amino acid sequence EQPFPEQPIPEQ (SEQ ID NO: 38); and
(p) the sixteenth peptide comprises the amino acid sequence PQPYPEQPQPFP (SEQ ID NO: 39).

20. The method of claim 19, wherein the composition comprises the first, second, third, fourth, fifth, sixth, seventh, eighth, ninth, tenth, eleventh, twelfth, thirteenth, fourteenth, fifteenth, and sixteenth peptides.

21. The method of any one of claims 9-20, wherein at least one of the peptides comprises an N-terminal pyroglutamate and/or a C-terminal amide group.

22. The method of claim 21, wherein each of the peptides comprises an N-terminal pyroglutamate and/or a C-terminal amide group.

23. The method of any one of claims 9-22, wherein each of the peptides is independently between 8 to 50 amino acids in length.

24. The method of any one of claims 1 to 23, wherein the method further comprises treating the subject if identified as having or at risk of having Celiac disease or providing information to the subject about a treatment.

25. The method of any one of claims 1 to 24, where the method further comprises a step of recommending a gluten-free diet if the subject is identified as having or at risk of having Celiac disease or providing information to the subject about such a diet.

26. The method of any one of claims 1 to 25, wherein the method further comprises performing other testing.

27. The method of claim 26, wherein the other testing comprises performing a serology assay, genotyping, and/or an intestinal biopsy.

28. The method of claims 1 to 27, wherein the subject is HLA-DQ2.5 positive, HLA-DQ2.2 positive and/or HLA-DQ8 positive.

29. The method of claim 28, wherein the subject is HLA-DQ2.5 positive.

30. The method of any one of claims 1 to 29, wherein the method further comprises administering a composition comprising wheat, rye, and/or barley, or a composition comprising a gluten peptide, to the subject at least once a day for one day.

31. The method of any one of claims 1 to 30, wherein the method further comprises administering a composition comprising wheat, rye, and/or barley to the subject at least once a day for two days.

32. The method of any one of claims 1 to 30, wherein the subject has not undergone a gluten challenge within 1 week of the sample being obtained from the subject.

33. The method of any one of claims 1 to 30, wherein the method further comprises administering a composition to the subject comprising:
10 micrograms of the first peptide and an equimolar amount of each of the second and third peptides;
15 micrograms of the first peptide and an equimolar amount of each of the second and third peptides;
20 micrograms of the first peptide and an equimolar amount of each of the second and third peptides; or
50 micrograms of the first peptide and an equimolar amount of each of the second and third peptides.

34. The method of claim 33, wherein the composition is administered to the subject once.

35. The method of any one of claims 1 to 34, wherein the subject has a level of IFN-gamma that is reduced or the same as a control level of IFN-gamma.

36. The method of claim 35, wherein the level of IFN-gamma is not statically significantly higher than the control level of IFN-gamma.

37. The method of claim 35 or 36, wherein the control level of IFN-gamma is 7.2 pg/mL.

38. The method of any one of claims 1 to 37, wherein the subject is on a diet that contains gluten.

39. A kit, comprising a binding partner for IP-10 and a composition comprising at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);
(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);
(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 40);
(h) an eighth peptide comprising the amino acid sequence PYPEQPQPF (SEQ ID NO: 22);
(i) a ninth peptide comprising the amino acid sequence PFPEQPEQI (SEQ ID NO: 42);
(j) a tenth peptide comprising the amino acid sequence EGSFQPSQE (SEQ ID NO: 15);
(k) an eleventh peptide comprising the amino acid sequence QGYYPTSPQ (SEQ ID NO: 16);
(l) a twelfth peptide comprising the amino acid sequence EQPEQPFPE (SEQ ID NO: 17) and the amino acid sequence EQPFPEQPQ (SEQ ID NO: 41);
(m) a thirteenth peptide comprising the amino acid sequence PFSEQEQPV (SEQ ID NO: 18);
(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 19);
(p) a sixteenth peptide comprising the amino acid sequence PFPQPELPY (SEQ ID NO: 1) and the amino acid sequence PQPELPYPY (SEQ ID NO: 43);
(q) a seventeenth peptide comprising the amino acid sequence EQPFPEQPI (SEQ ID NO: 20) and the amino acid sequence PFPEQPIPE (SEQ ID NO: 21); and
(r) an eighteenth peptide comprising the amino acid sequence PQPYPEQPQ (SEQ ID NO: 23) and the amino acid sequence PYPEQPQPF (SEQ ID NO: 22).

40. The kit of claim 39, wherein
(a) the first peptide comprises the amino acid sequence LQPFPQPELPYPQPQ (SEQ ID NO: 45);
(b) the second peptide comprises the amino acid sequence
(SEQ ID NO: 46)
QPFPQPEQPFPWQP;
(c) the third peptide comprises the amino acid sequence PEQPIPEQPQPYPQQ (SEQ ID NO: 47);
(d) the fourth peptide comprises the amino acid sequence
(SEQ ID NO: 48)
QPFPQPEQPIPVQPEQS;
(e) the fifth peptide comprises the amino acid sequence
(SEQ ID NO: 49)
QPFPQPEQPTPIQPEQP;
(f) the sixth peptide comprises the amino acid sequence
(SEQ ID NO: 50)
QPFPQPEQPFPLQPEQP;
(g) the seventh peptide comprises the amino acid sequence QPFPQPEQPFSQQ (SEQ ID NO: 51);
(h) the eighth peptide comprises the amino acid sequence PQPYPEQPQPFPQQ (SEQ ID NO: 52);
(i) the ninth peptide comprises the amino acid sequence QPFPEQPEQIIPQQP (SEQ ID NO: 53);
(j) the tenth peptide comprises the amino acid sequence SGEGSFQPSQENPQ (SEQ ID NO: 54);
(k) the eleventh peptide comprises the amino acid sequence
(SEQ ID NO: 55)
GQQGYYPTSPQQSG;
(l) the twelfth peptide comprises the amino acid sequence PEQPEQPFPEQPQQ (SEQ ID NO: 56);
(m) the thirteenth peptide comprises the amino acid sequence
(SEQ ID NO: 57)
QPPFSEQEQPVLPQ;
(n) the fourteenth peptide comprises the amino acid sequence
(SEQ ID NO: 44)
PEQPFPEQPIPEQPQPYP;
(o) the fifteenth peptide comprises the amino acid sequence
(SEQ ID NO: 58)
QPYPQPELPYPQPQ;
(p) the sixteenth peptide comprises the amino acid sequence
(SEQ ID NO: 59)
QPFPQPELPYPYPQ;
(q) the seventeenth peptide comprises the amino acid sequence
(SEQ ID NO: 60)
PQEQPFPEQPIPEQP;
and
(r) the eighteenth peptide comprises the amino acid sequence
(SEQ ID NO: 61)
QPQPYPEQPQPFPQQ.

41. The kit of claim 39 or 40, wherein the composition comprises the first, second, and third peptides.

42. The kit of claim 41, wherein each of the peptides are present in an amount of 5 ug/mL in the composition.

43. The kit of claim 41, wherein each of the peptides are present in an amount of 10 ug/mL in the composition.

44. The kit of claim 41, wherein each of the peptides are present in an amount of 20 ug/mL in the composition.

45. The kit of claim 41, wherein each of the peptides are present in an amount of 50 ug/mL in the composition.

46. The kit of claim 41, wherein each of the peptides are present in an amount of 5 uM in the composition.

47. The kit of claim 41, wherein each of the peptides are present in an amount of 10 uM in the composition.

48. The kit of claim 41, wherein each of the peptides are present in an amount of 25 uM in the composition.

49. The kit of claim 41, wherein each of the peptides are present in an amount of 50 uM in the composition.

50. The kit of claim 39 or 40, wherein the composition comprises:
(a) the first, second, third, fourth, fifth, sixth, seventh, eighth, ninth, tenth, eleventh, twelfth, and thirteenth peptides;
(b) the second, fourth, fifth, sixth, seventh, eighth, ninth, tenth, eleventh, twelfth, thirteenth, fourteenth, fifteenth, and sixteenth peptides; or
(c) the first, second, third, fourth, fifth, sixth, tenth, eleventh, twelfth, thirteenth, fifteenth, seventeenth, and eighteenth peptides.

51. The kit of claim 50, wherein each of the peptides are present in an amount of 2.5 ug/mL in the composition.

52. The kit of claim 50, wherein each of the peptides are present in an amount of 5 ug/mL in the composition.

53. The kit of claim 50, wherein each of the peptides are present in an amount of 10 ug/mL in the composition.

54. The kit of claim 50, wherein each of the peptides are present in an amount of 25 ug/mL in the composition.

55. The kit of claim 50, wherein each of the peptides are present in an amount of 5 uM in the composition.

56. The kit of claim 50, wherein each of the peptides are present in an amount of 10 uM in the composition.

57. The kit of claim 50, wherein each of the peptides are present in an amount of 25 uM in the composition.

58. The kit of claim 50, wherein each of the peptides are present in an amount of 50 uM in the composition.

59. The kit of any one of claims 39 to 58, wherein each of the peptides comprises an N-terminal pyroglutamate and/or a C-terminal amide group.

60. A kit, comprising a binding partner for IP-10 and a composition comprising at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7) and the amino acid sequence PQPEQPIPV (SEQ ID NO: 8);
(e) a fifth peptide comprising the amino acid sequence EQPIPVQPE (SEQ ID NO: 9);
(f) a sixth peptide comprising the amino acid sequence PFPQPEQPT (SEQ ID NO: 10) and the amino acid sequence PQPEQPTPI (SEQ ID NO: 11);
(g) a seventh peptide comprising the amino acid sequence EQPTPIQPE (SEQ ID NO: 12);
(h) an eighth peptide comprising the amino acid sequence PQPEQPFPL (SEQ ID NO: 13);
(i) a ninth peptide comprising the amino acid sequence EQPFPLQPE (SEQ ID NO: 14);
(j) a tenth peptide comprising the amino acid sequence EGSFQPSQE (SEQ ID NO: 15);
(k) a eleventh peptide comprising the amino acid sequence QGYYPTSPQ (SEQ ID NO: 16);
(l) a twelfth peptide comprising the amino acid sequence EQPEQPFPE (SEQ ID NO: 17);
(m) a thirteenth peptide comprising the amino acid sequence PFSEQEQPV (SEQ ID NO: 18);
(n) a fourteenth peptide comprising the amino acid sequence PYPQPELPY (SEQ ID NO: 19);
(o) a fifteenth peptide comprising the amino acid sequence EQPFPEQPI (SEQ ID NO: 20) and the amino acid sequence PFPEQPIPE (SEQ ID NO: 21); and
(p) a sixteenth peptide comprising the amino acid sequence PYPEQPQPF (SEQ ID NO: 22) and the amino acid sequence PQPYPEQPQ (SEQ ID NO: 23).

61. The kit of claim 60, wherein
(a) the first peptide comprises the amino acid sequence PFPQPELPYPQP (SEQ ID NO: 24);
(b) the second peptide comprises the amino acid sequence PFPQPEQPFPWQ (SEQ ID NO: 25);
(c) the third peptide comprises the amino acid sequence EQPIPEQPQPYP (SEQ ID NO: 26);
(d) the fourth peptide comprises the amino acid sequence PFPQPEQPIPVQ (SEQ ID NO: 27);
(e) the fifth peptide comprises the amino acid sequence PEQPIPVQPEQS (SEQ ID NO: 28);
(f) the sixth peptide comprises the amino acid sequence PFPQPEQPTPIQ (SEQ ID NO: 29);
(g) the seventh peptide comprises the amino acid sequence PEQPTPIQPEQP (SEQ ID NO: 30);
(h) the eighth peptide comprises the amino acid sequence PFPQPEQPFPLQ (SEQ ID NO: 31);
(i) the ninth peptide comprises the amino acid sequence PEQPFPLQPEQP (SEQ ID NO: 32);
(j) the tenth peptide comprises the amino acid sequence GEGSFQPSQENP (SEQ ID NO: 33);
(k) the eleventh peptide comprises the amino acid sequence QQGYYPTSPQQS (SEQ ID NO: 34);
(l) the twelfth peptide comprises the amino acid sequence PEQPEQPFPEQP (SEQ ID NO: 35);
(m) the thirteenth peptide comprises the amino acid sequence PPFSEQEQPVLP (SEQ ID NO: 36);
(n) the fourteenth peptide comprises the amino acid sequence PYPQPELPYPQP (SEQ ID NO: 37);
(o) the fifteenth peptide comprises the amino acid sequence EQPFPEQPIPEQ (SEQ ID NO: 38); and
(p) the sixteenth peptide comprises the amino acid sequence PQPYPEQPQPFP (SEQ ID NO: 39).

62. The kit of claim 60 or 61, wherein the composition comprises the first, second, third, fourth, fifth, sixth, seventh, eighth, ninth, tenth, eleventh, twelfth, thirteenth, fourteenth, fifteenth, and sixteenth peptides.

63. The kit of claim 62, wherein each of the peptides are present in an amount of 5 ug/mL in the composition.

64. The kit of claim 62, wherein each of the peptides are present in an amount of 10 ug/mL in the composition.

65. The kit of claim 62, wherein each of the peptides are present in an amount of 20 ug/mL in the composition.

66. The kit of claim 62, wherein each of the peptides are present in an amount of 50 ug/mL in the composition.

67. The kit of claim 62, wherein each of the peptides are present in an amount of 5 uM in the composition.

68. The kit of claim 62, wherein each of the peptides are present in an amount of 10 uM in the composition.

69. The kit of claim 62, wherein each of the peptides are present in an amount of 25 uM in the composition.

70. The kit of claim 62, wherein each of the peptides are present in an amount of 50 uM in the composition.

71. The kit of any one of claims 60 to 70, wherein each of the peptides comprises an N-terminal pyroglutamate and/or a C-terminal amide group.

72. A kit, comprising a binding partner for IP-10 and a composition comprising at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence PIPEQPQPY (SEQ ID NO: 6);
(d) a fourth peptide comprising the amino acid sequence PFPQPEQPIP (SEQ ID NO: 62) and the amino acid sequence EQPIPVQPE (SEQ ID NO: 9);
(e) a fifth peptide comprising the amino acid sequence PFPQPEQPTPI (SEQ ID NO: 63) and the amino acid sequence EQPTPIQPE (SEQ ID NO: 12);
(f) a sixth peptide comprising the amino acid sequence PQPEQPFPL (SEQ ID NO: 13) and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 40);
(h) an eighth peptide comprising the amino acid sequence PYPEQPQPF (SEQ ID NO: 22);
(i) a ninth peptide comprising the amino acid sequence PFPEQPEQIIP (SEQ ID NO: 64);
(j) a tenth peptide comprising the amino acid sequence EGSFQPSQE (SEQ ID NO: 15);
(k) an eleventh peptide comprising the amino acid sequence QGYYPTSPQ (SEQ ID NO: 16);
(l) a twelfth peptide comprising the amino acid sequence EQPEQPFPEQPQ (SEQ ID NO: 65);
(m) a thirteenth peptide comprising the amino acid sequence PFSEQEQPV (SEQ ID NO: 18);
(n) a fourteenth peptide comprising the amino acid sequence EQPFPEQPI (SEQ ID NO: 20) and PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 19);
(p) a sixteenth peptide comprising the amino acid sequence PFPQPELPY (SEQ ID NO: 1) and the amino acid sequence PQPELPYPY (SEQ ID NO: 43); and
(q) a seventeenth peptide comprising the amino acid sequence EQPFPEQPI (SEQ ID NO: 20).

73. The kit of claim 72, wherein
(a) the first peptide comprises the amino acid sequence LQPFPQPELPYPQPQ (SEQ ID NO: 45);
(b) the second peptide comprises the amino acid sequence QPFPQPEQPFPWQP (SEQ ID NO: 46);
(c) the third peptide comprises the amino acid sequence PEQPIPEQPQPYPQQ (SEQ ID NO: 47);
(d) the fourth peptide comprises the amino acid sequence QPFPQPEQPIPVQPEQS (SEQ ID NO: 48);
(e) the fifth peptide comprises the amino acid sequence QPFPQPEQPTPIQPEQP (SEQ ID NO: 49);
(f) the sixth peptide comprises the amino acid sequence QPFPQPEQPFPLQPEQP (SEQ ID NO: 50);
(g) the seventh peptide comprises the amino acid sequence QPFPQPEQPFSQQ (SEQ ID NO: 51);
(h) the eighth peptide comprises the amino acid sequence PQPYPEQPQPFPQQ (SEQ ID NO: 52);
(i) the ninth peptide comprises the amino acid sequence QPFPEQPEQIIPQQP (SEQ ID NO: 53);
(j) the tenth peptide comprises the amino acid sequence SGEGSFQPSQENPQ (SEQ ID NO: 54);
(k) the eleventh peptide comprises the amino acid sequence GQQGYYPTSPQQSG (SEQ ID NO: 55);
(l) the twelfth peptide comprises the amino acid sequence PEQPEQPFPEQPQQ (SEQ ID NO: 56);
(m) the thirteenth peptide comprises the amino acid sequence QPPFSEQEQPVLPQ (SEQ ID NO: 57);
(n) the fourteenth peptide comprises the amino acid sequence PEQPFPEQPIPEQPQPYP (SEQ ID NO: 44);
(o) the fifteenth peptide comprises the amino acid sequence QPYPQPELPYPQPQ (SEQ ID NO: 58);
(p) the sixteenth peptide comprises the amino acid sequence QPFPQPELPYPYPQ (SEQ ID NO: 59); and
(q) the seventeenth peptide comprises the amino acid sequence EQPFPEQPI (SEQ ID NO: 20).

74. The kit of claim 72 or 73, wherein the composition comprises the first, second, and third peptides.

75. The kit of claim 74, wherein each of the peptides are present in an amount of 5 ug/mL in the composition.

76. The kit of claim 74, wherein each of the peptides are present in an amount of 10 ug/mL in the composition.

77. The kit of claim 74, wherein each of the peptides are present in an amount of 20 ug/mL in the composition.

78. The kit of claim 74, wherein each of the peptides are present in an amount of 50 ug/mL in the composition.

79. The kit of claim 72 or 73, wherein the composition comprises the first, second, third, fourth, fifth, sixth, seventh, eighth, ninth, tenth, eleventh, twelfth, and thirteenth peptides or the composition comprises the second, fourth, fifth, sixth, seventh, eighth, ninth, tenth, eleventh, twelfth, thirteenth, fourteenth, fifteenth, and sixteenth peptides.

80. The kit of claim 79, wherein each of the peptides are present in an amount of 2.5 ug/mL in the composition.

81. The kit of claim 79, wherein each of the peptides are present in an amount of 5 ug/mL in the composition.

82. The kit of claim 79, wherein each of the peptides are present in an amount of 10 ug/mL in the composition.

83. The kit of claim 79, wherein each of the peptides are present in an amount of 25 ug/mL in the composition.

84. The kit of any one of claims 72 to 83, wherein each of the peptides comprises an N-terminal pyroglutamate and/or a C-terminal amide group.

85. The kit of any one of claims 39 to 84, wherein the kit further comprises a second composition comprising the first, second and third peptides, wherein the second composition contains
10 micrograms of the first peptide and an equimolar amount of each of the second and third peptides;
15 micrograms of the first peptide and an equimolar amount of each of the second and third peptides;
20 micrograms of the first peptide and an equimolar amount of each of the second and third peptides; or
50 micrograms of the first peptide and an equimolar amount of each of the second and third peptides.
